(19) United States
(12) Patent Application Publication
(10) Pub. No.: US 2024/0008880 A1
(43) Pub. Date: Jan. 11, 2024

(54) APPARATUS, SYSTEM, AND METHOD FOR PATIENT-SPECIFIC SYSTEMS, METHODS, AND INSTRUMENTATION

(71) Applicant: Treace Medical Concepts, Inc., Ponte Vedra, FL (US)

(72) Inventors: James Q. SPITLER, Winter Garden, FL (US); Adam D. PERLER, St. Petersburg, FL (US); Lee M. HLAD, Waycross, GA (US)

(21) Appl. No.: 18/220,226

(22) Filed: Jul. 10, 2023

Related U.S. Application Data
(60) Provisional application No. 63/388,171, filed on Jul. 11, 2022.

(51) Int. Cl. A61B 17/15; A61B 17/17; G06T 7/00

(52) U.S. Cl. CPC A61B 17/151; A61B 17/157; A61B 17/1775; G06T 7/0012; G06T 2207/30008

(57) ABSTRACT

An apparatus, system, and method are disclosed for remediating a condition present in a patient. In some implementations, the apparatus may include a resection guide having: a body having an anterior side, a posterior side, a medial side, a lateral side, a superior side, and an inferior side. The resection guide also includes a first resection feature configured to guide a cutting tool to form a first osteotomy in a first bone. The first osteotomy is defined based at least partially on user directions and at least partially on a bone model of at least a portion of the first bone. The bone model is based on medical imaging of a patient's foot. The resection guide includes bone attachment feature configured to secure the resection guide to the first bone.

### CROSS-REFERENCE TO RELATED APPLICATIONS

This application claims the benefit of U.S. Provisional Application No. 63/388,171, filed Jul. 11, 2022, which is hereby incorporated by reference in its entirety.

### TECHNICAL FIELD

The present disclosure relates to surgical devices, systems, instruments, and methods. More specifically, the present disclosure relates to patient-specific instruments, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to accurately locate, position, deploy, and/or orient one or more osteotomy cuts, fixation guides, fixators, bone tunnels, implants, points of attachment for ends of grafts or soft tissue, and the like. Determining and locating an optimal location and trajectory for one or more steps of the surgical procedures and/or securing instruments that can guide or assist in steps of the surgical procedures such as performing osteotomies, deploying fixation and/or implants, and the like, can be challenging, given conventional techniques and instruments.

One of the challenges with conventional techniques is how to translate, map, or convert from a model of a patient's anatomy and/or virtual instrumentation to the real, physical world for performing a surgical procedure. Furthermore, surgical procedures can be extra challenging when working on anatomy such as bones of a patient's ankle, foot, or hand which have unique surface configurations, landmarks, and/or deformities that called for extra accuracy and/or precision. In certain surgical procedures such as a joint fusion, one goal may be to minimize the amount of bone removed in order to successfully fuse the joint. Accomplishing this goal can require extra precision and accuracy in resecting the bone(s), reducing the bones, and/or deploying fixation to achieve a successful fusion.

What is needed is one or more instruments to facilitate locating, aligning, orienting, planning, mapping from virtual models to physical anatomy, preparing for, initiating, executing, and/or completing such surgical procedures. In addition, what is needed is methods, apparatus, systems, implants and/or instrumentation that is customized to a specific patient. In addition, what is needed is methods, apparatus, systems, implants and/or instrumentation that includes direct input from the surgeon to perform a surgical procedure customized to a particular patient. Existing solutions for guiding orthopedic surgical procedures are inadequate and error prone.

### SUMMARY

The various apparatus, devices, systems, and/or methods of the present disclosure have been developed in response to the present state of the art, and in particular, in response to the problems and needs in the art that have not yet been fully solved by currently available technology.

One general aspect of the present disclosure may include an apparatus that may include a resection guide having: a body having an anterior side, a posterior side, a medial side, a lateral side, a superior side, and an inferior side; a first resection feature configured to guide a cutting tool to form a first osteotomy in a first bone, the first osteotomy defined based at least partially on user directions and at least partially on a bone model of at least a portion of the first bone, the bone model based on medical imaging of a patient's foot; and a first bone attachment feature configured to secure the resection guide to the first bone.

Implementations may also include one or more of the following features. An apparatus may include: a bone engagement feature configured to engage with at least a portion of the first bone at a position that substantially matches a model position of a model of the resection guide engaging the bone model. An apparatus where the bone engagement feature may include: a bone engagement surface configured to at least partially match a contour of a surface of the first bone when the resection guide is positioned for use; and a body section extending from the body to support the bone engagement surface. An apparatus where the bone engagement feature may include a bone probe configured to at least partially engage with a landmark associated with the first bone. A 5 where the bone engagement feature is configured based at least partially on the user directions and at least partially on the bone model. An apparatus may include a plurality of bone engagement features, at least one of the plurality of bone engagement features configured based on the user directions. An apparatus may include: a second resection feature coupled to the body and configured to guide the cutting tool to form a second osteotomy in a second bone, the second osteotomy shaped to form a resection interface with the first osteotomy; a second bone attachment feature configured to secure the resection guide to the second bone; and where the second bone is part of a joint that includes the first bone.

One general aspect of the present disclosure may include a system that may include a tibial resection guide having: a body having an anterior side, a posterior side, a medial side, a lateral side, a superior side, and an inferior side; a tibia resection feature configured to guide a cutting tool to form a first osteotomy in a tibia, the tibia resection feature extending through the tibial resection guide from the anterior side to the posterior side at a position at least partially determined based on a bone model of at least a portion of the patient's foot, the bone model based on medical imaging of the patient's foot; a tibial bone attachment feature configured to secure the tibial resection guide to the tibia. A system that may also include a talus resection guide having: a body having an anterior side, a posterior side, a medial side, a lateral side, a superior side, and an inferior side; a talus resection feature configured to guide the cutting tool to form a second osteotomy in a talus, the talus resection feature extending through the talus resection guide from the anterior side to the posterior side at a position at least partially determined based on the bone model, the second osteotomy configured to cooperate with the first osteotomy to form a resection interface between the tibia and the talus; and a talus bone attachment feature configured to secure the talus resection guide the talus.

Implementations may also include one or more of the following features. A system where the resection interface may include a resected distal end of the tibia and a resected proximal end of the talus. A system where the resection interface may include a polygonal cross-section taken along an anterior-posterior axis that extends from the anterior side to the posterior side of the body. A system where the resection interface may include a curve shape cross-section taken along an anterior-posterior axis that extends from the anterior side to the posterior side of the body. A system may include an alignment guide coupled to one of the tibial resection guide and the talus resection guide, the alignment guide configured to indicate an orientation of one of the tibial resection guide and the talus resection guide relative to a mechanical axis of the tibia. A system where: the tibial resection guide may include a tibial bone engagement feature having a bone engagement surface configured to register to a surface of the tibia; and the talus resection guide may include a talus bone engagement feature having a bone engagement surface configured to register to a surface of the talus. A system where at least one of the tibial bone engagement feature and the talus bone engagement feature may include a body section that is coupled to and supports the bone engagement surface and where the body section is configured based at least partially on user directions. A system may include a positioning guide configured to cooperate with one of the tibial bone attachment feature and the talus bone attachment feature to abut the first osteotomy against the second osteotomy in a stable relationship to close the resection interface. A system may include a set of stops configured to prevent the cutting tool from cutting tissue beyond a boundary defined at least partially using the bone model. A system where the system includes the set of stops based on user directions. A system where one of the tibial resection guide and the talus resection guide may include a fastener guide configured to guide a fixation system that fixes the tibia to the talus.

One general aspect of the present disclosure may include a method that may include positioning a tibial resection guide onto an anterior surface of a distal end of a tibia, the tibial resection guide having: a body having an anterior side, a posterior side, a medial side, a lateral side, a superior side, and an inferior side; a tibia resection feature configured to guide a cutting tool to prepare the tibia for fusion to a talus; a tibial bone attachment feature configured to secure the tibial resection guide to the tibia; a bone engagement feature having a bone engagement surface configured to at least partially match a contour of a portion of the anterior surface of the distal end of the tibia when the tibial resection guide is positioned for use; where the tibial resection guide is defined based at least partially on user directions and at least partially on a bone model of at least a portion of the tibia, the bone model based on medical imaging of a patient's foot.

A method that may also include deploying a set of fasteners as part of the tibial bone attachment feature to secure the tibial resection guide to the tibia. A method that may furthermore include deploying an alignment guide that includes a shaft directed towards a proximal end of the tibia. A method that may in addition include inserting the cutting tool into the tibia resection feature and cutting the tibia to form a first osteotomy. A method that may moreover include positioning a talus resection guide onto an anterior surface of the proximal end of the talus, the talus resection guide having: a body having an anterior side, a posterior side, a medial side, a lateral side, a superior side, and an inferior side; a talus resection feature configured to guide the cutting tool to prepare the talus for fusion to the tibia; a talus bone attachment feature configured to secure the talus resection guide to the talus; a bone engagement feature having a bone engagement surface configured to at least partially match a contour of a portion of the anterior surface of the proximal end of the talus when the talus resection guide is positioned for use; where the talus resection guide is defined based at least partially on user directions and at least partially on a bone model of at least a portion of the talus, the bone model based on medical imaging of the patient's foot. A method that may also include deploying a set of fasteners as part of the talus bone attachment feature to secure the talus resection guide to the talus. A method that may furthermore include inserting the cutting tool into the talus resection feature and cutting the talus to form a second osteotomy. A method that may in addition include deploying fixation across the first osteotomy and the second osteotomy to enable fusion of the tibia and the talus.

Implementations may also include one or more of the following features. A method may include: accessing an anterior surface of a distal end of a tibia and an anterior surface of a proximal end of a talus of a patient's foot; deploying a set of stops within the tibia resection feature to manage the cutting tool; verifying the position of the tibial resection guide by comparing the shaft to a mechanical axis of the tibia and a set of fasteners deployed using the tibial resection guide; and reducing the first osteotomy and the second osteotomy by abutting a resected distal end of the tibia and a resected proximal end of the talus.

One general aspect of the present disclosure may include a system that may include a first resection guide having: a body having an anterior side, a posterior side, a medial side, a lateral side, a superior side, and an inferior side; a first resection feature configured to guide a cutting tool to form a curved osteotomy in a sagittal plane of an ankle of a patient, the curved osteotomy at least partially determined based on a bone model of at least a portion of the patient's ankle, the bone model based on medical imaging of the patient's ankle; a first bone attachment feature configured to secure the first resection guide to at least one bone of the patient. A system that may also include a second resection guide having: a body having an anterior side, a posterior side, a medial side, a lateral side, a superior side, and an inferior side; a second resection feature configured to guide a cutting tool to form an angled curved osteotomy, the angled curved osteotomy having a curve in a sagittal plane of an ankle of a patient that extends at a first angle in a frontal plane of the ankle that is not perpendicular to a mechanical axis of a tibia of the patient, the angled curved osteotomy at least partially determined based on a bone model of at least a portion of the patient's ankle, the bone model based on medical imaging of the patient's ankle; and a second bone attachment feature configured to secure the second resection guide to at least one bone of the patient.

Implementations may also include one or more of the following features. A system where the curved osteotomy extends in the frontal plane at a second angle that is perpendicular to the mechanical axis of the tibia of the patient and where the first angle and the second angle are determined at least partially based on the bone model of the portion of the patient's ankle. A system where the first angle and the second angle are confirmed by a surgeon. A system where the first angle and the second angle are configured such that reduction of the curved osteotomy and the angled curved osteotomy remediates a deformity of the patient. A system where the first resection feature extends perpendicularly from the medial side of the first resection guide to the lateral side of the first resection guide at the second angle and the second resection feature extends from the medial side of the second resection guide to the lateral side of the second resection guide at the first angle. A system where at least one of the first resection feature and the second resection feature may include one of a curved slot and a plurality of openings arranged in a curved pattern. A system where at least one of the first resection guide and the second resection guide may include a bone engagement feature having: an insert configured to extend into the curved osteotomy; and a bone engagement surface configured to engage with bone on one or both sides of the curved osteotomy. A system where at least one of the first resection guide and the second resection guide may include a first bone engagement surface on one side and a second bone engagement surface on another side.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments, according to one embodiment.

FIG. 4 illustrates an exemplary system configured to generate one or more patient-specific instruments, according to one embodiment.

FIG. 5 illustrates an exemplary system configured to generate one or more patient-specific instruments, according to one embodiment.

FIG. 6 illustrates an exemplary system configured to generate a patient-specific osteotomy system, according to one embodiment.

FIG. 7 illustrates an exemplary system for remediating a condition present in a patient's foot, according to one embodiment.

FIG. 8 illustrates an exemplary system for an osteotomy, according to one embodiment.

FIG. 9A-9F illustrate views of a resection guide of the osteotomy system of FIG. 8, according to one embodiment.

FIG. 9G illustrates a cross section view of the resection guide of FIG. 9F taken along line 9G, according to one embodiment.

FIG. 9H illustrates a cross section view of the resection guide in FIG. 9F taken along line 9H, according to one embodiment.

FIGS. 10A-10F illustrate views of a resection guide of the osteotomy system of FIG. 8, according to one embodiment.

FIGS. 11A-11F illustrate views of a resection guide of an osteotomy system, according to an alternative embodiment.

FIGS. 12A-12C illustrate different views a surgical osteotomy procedure using the osteotomy system of FIG. 8, according to one embodiment.

FIG. 12D illustrates resection guides, according to another embodiment.

FIGS. 13A-13F illustrate alternative embodiments of a resection interface between two osteotomies using an exemplary osteotomy system, according to one embodiment.

FIGS. 14A-14B illustrate views of resection guides of an osteotomy system, according to alternative embodiments.

FIG. 15A illustrates a lateral side view of a resection guide of an osteotomy system, according to one embodiment.

FIG. 15B illustrates an anterior view of a positioning guide of an osteotomy system, according to one embodiment.

FIG. 16 is a flowchart diagram depicting a method for remediating a bone condition, according to one embodiment.

FIGS. 17A-17E illustrate different views of one or more stages in a surgical procedure that includes one or more embodiments of the present disclosure.

FIG. 18 illustrates an exemplary system for an osteotomy using a lateral approach, according to one embodiment.

FIG. 19A-19G illustrate views of a resection guide of the osteotomy system of FIG. 18, according to one embodiment.

FIG. 20A-20H illustrate views of a resection guide of the osteotomy system of FIG. 18, according to one embodiment.

FIGS. 21A-21E illustrate different views of a surgical osteotomy procedure using the osteotomy system of FIG. 18, according to one embodiment.

FIG. 22 illustrates an exemplary system for an osteotomy using a medial approach, according to one embodiment.

FIG. 23A-23G illustrate views of a resection guide of the osteotomy system of FIG. 22, according to one embodiment.

FIG. 24A-24G illustrate views of a resection guide of the osteotomy system of FIG. 22, according to one embodiment.

FIGS. 25A-25E illustrate different views of a surgical osteotomy procedure using the osteotomy system of FIG. 22, according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

“Patient specific” refers to a feature, an attribute, a characteristic, a structure, function, structure, device, guide, tool, instrument, apparatus, member, component, system, assembly, module, or subsystem or the like that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific attribute or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, a trajectory for an instrument, implant, or anatomical part of a patient, a lateral offset, and/or other features.

“Patient-specific instrument” refers to an instrument, implant, or guide designed, engineered, and/or fabricated for use with a specific patient. In one aspect, a patient-specific instrument is unique to a patient and may include features unique to the patient such as a surface contour or other features.

“Patient-specific positioning guide” or “Patient-specific positioner” refers to an instrument, implant, positioner, structure, or guide designed, engineered, and/or fabricated for use as a positioner with a specific patient. In one aspect, a patient-specific positioning guide is unique to a patient and may include features unique to the patient such as patient-specific offsets, translation distances, openings, angles, orientations, anchor a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient-specific trajectory guide” refers to a trajectory guide designed, engineered, and/or fabricated for use with a specific patient. In one aspect, a patient-specific trajectory guide is unique to a single patient and may include features unique to the patient such as a surface contour or other features.

“Patient specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

As used herein, a “handle” or “knob” refers to a structure used to hold, control, or manipulate a device, apparatus, component, tool, or the like. A “handle” may be designed to be grasped and/or held using one or two hands of a user. In certain embodiments, a handle or knob may be an elongated structure. In one embodiment, a knob may be a shorter stubby structure.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

“Bone engagement feature” refers to a structure, feature, component, aspect configured to contact, touch, abut, and/or engage with a bone, a bone part, and/or a bone fragment. A bone engagement feature may enable temporary engagement with a bone or bone fragment or permanent engagement with a bone or bone fragment. A bone engagement feature may include a bone engagement surface and a body section that supports the bone engagement surface. In certain embodiments, a bone engagement feature may include a bone probe. In one embodiment, a bone engagement feature may include a landmark registration feature.

“Frangible” refers to a type of material designed, engineered, and/or configured to break easily under an expected force. Frangible objects may be designed to break easily under the expected force to provide a safety feature, a convenience feature, or the like. Frangible objects can be made from metal, plastic, ceramics, wood, paper, or the like. Frangible also includes something that is breakable or fragile; especially something that is intentionally made so. (Search “frangible” on wordhippo.com. WordHippo, 2023. Web. Accessed 11 May 2023. Modified.)

As used herein, “side” refers to a structure or part of a structure including, but not limited to: one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Tissue” refers to a structure that makes up a one or more anatomical structures of a patient (i.e., human or animal). Tissue can be soft tissue or hard tissue. “Soft tissue” refers to tissue of a patient (i.e., human or animal). Examples of soft tissue include but are not limited to skin, ligament, tendon, fascia, fat muscle, fibrous tissue, blood vessels, lymph vessels, brain tissue, and/or nerves. “Hard tissue” refers to any human or animal tissue that is not soft tissue. Examples of hard tissue include bone, teeth, tooth enamel, dentin, cementum, cartilage, or the like.

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed February 2022.)

“Boundary” refers to a structure, line, or area where an object, surface, line, area, or operation is or is expected to begin and/or end. A boundary can be similar to a border.

“Landmark registration feature” or “Landmark” refers to a structure configured to engage with a feature, aspect, attribute, or characteristic of a first object to orient and/or position a second object that includes the landmark registration feature with respect to the first object. A variety of structures can serve as a landmark registration feature. For example, a landmark registration feature may include a protrusion, a projection, a tuberosity, a cavity, a void, a divot, a tab, an extension, a hook, a curve, or the like. In the context of bones of a patient a landmark registration feature can include any protuberance, void, divot, concave section, sesamoid, bone spur or other feature on, or extending from, a bone of a patient. A landmark refers to any structure of an anatomical structure that is referenced, contacted, engaged with and/or associated with a landmark registration feature.

“Probe bone engagement surface” refers to a bone engagement surface on one surface of a probe or part of a probe.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

As used herein, “patient-specific osteotomy procedure” refers to an osteotomy procedure that has been adjusted, tailored, modified, or configured to specifically address the needs or desires or a particular patient. In certain aspects, one patient-specific osteotomy procedure may be useable in connection with only one patient. In other aspects, one patient-specific osteotomy procedure may be useable with a number of patients having a particular class of characteristics.

“Ankle fusion procedure” refers to a surgical procedure that seeks to immobilize an ankle joint of a patient. The surgery fuses two or more bones of the ankle of the patient. The surgery involves the use of screws, plates, medical nails, and other hardware or fasteners to achieve bone union. Ankle fusion is considered to be the gold standard for treatment of end-stage ankle arthritis. Ankle fusion trades joint mobility for relief from pain. (Search “ankle fusion” on Wikipedia.com Dec. 21, 2022. CC-BY-SA 3.0 Modified. Accessed Jun. 28, 2023.) An ankle fusion procedure may also be referred to as ankle arthrodesis, talocrural joint fusion, tibiotalar arthrodesis, and tibiotalocalcaneal arthrodesis. An ankle fusion procedure can be performed using a variety of approaches to the ankle including an anterior approach, a posterior approach, a lateral approach and a medial approach. Each approach may use common or different instrumentation or implants for the procedure.

“Deformity” refers to any abnormality in or of an organism, a part of an organism, or an anatomical structure of a patient that appears or functions differently than is considered normal, or is common, in relation to the same organism, a part of an organism, or an anatomical structure of other subjects of the same species as the patient. (Search “deformity” on Wikipedia.com Jun. 13, 2023. CC-BY-SA 3.0 Modified. Accessed Jun. 28, 2023.)

“Prescription” or “Prescribed” refers to an instruction, request, direction, determination, designation, authorization, and/or order, as by a physician or nurse practitioner, for the administration of a medicine, preparation of an implant, preparation of an instrument, or other intervention. Often a prescription is written. Prescription can also refer to the prescribed medicine or intervention. (Search “prescription” on wordhippo.com. WordHippo, 2023. Web. Accessed 3 May 2023. Modified.)

“User directions” refers to any request, instruction, direction, input, feedback, prescription, designation, order, directive, or the like from a user of an apparatus, system, device, component, subsystem, or other object. User directions can be created, sent, and/or received in a variety of forms and/or formats, including, but not limited to, a user action in a user interface, a prescription, a form, a conversation, an electronic mail message, a text message, a gesture by the user, or the like. In the context of an osteotomy procedure, user directions can include a set of default settings or choices or instructions for fabrication of a patient-specific instrument or set of instruments, an online form completed by a user (e.g., surgeon), a set of modifications to an original set of user directions, and the like.

“Position” refers to a place or location. (Search “position” on wordhippo.com. WordHippo, 2022. Web. Modified. Accessed 9 Aug. 2022.) Often, a position refers to a place or location of a first object in relation to a place or location of another object. One object can be positioned on, in, or relative to a second object. In addition, a position can refer to a place or location of a first object in relation to a place or location of another object in a virtual environment. For example a model of one object can be positioned relative to a model of another object in a virtual environment such as a modeling software program.

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly. In one embodiment, a stop may be used to manage and/or control a cutting tool.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like. Fixation refers to the act of deploying or using a fixator to fix two structures together.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. Often, an anchor is coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. In certain embodiments, the term anchor may be used as an adjective that describes a function, feature, or purpose for the noun the adjective ‘anchor’ describes. For example, an anchor hole is a hole that serves as or can be used as an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited to any fastener.

“Clearance” refers to a space or opening that provides an unobstructed area to permit one object to move freely in relation to another object.

“Correction,” in a medical context, refers to a process, procedure, device, instrument, apparatus, system, implant, or the like that is configured, designed, developed, fabricated, configured, and/or organized to adjust, translate, move, orient, rotate, or otherwise change an anatomical structure from an original position, location, and/or orientation to a new position, location, and/or orientation that provides a benefit to a patient. The benefit may be one of appearance, anatomical function, pain relief, increased mobility, increased strength, and the like.

“Uniplanar correction” refers to a medical correction, which can include an osteo correction, in one plane (e.g., one of a sagittal plane, a transverse plane, and a coronal/frontal plane) of an anatomical structure such as a foot, hand, or body of a patient.

“Biplanar correction” refers to a medical correction, which can include an osteo correction, in two planes (e.g., two of a sagittal plane, a transverse plane, and a coronal/frontal plane) of an anatomical structure such as a foot, hand, or body of a patient.

“Triplane correction” refers to a medical correction, which can include an osteo correction, in three planes (e.g., all three planes of a sagittal plane, a transverse plane, and a coronal/frontal plane) of an anatomical structure such as a foot, hand, or body of a patient.

“Probe” refers to a medical instrument used to explore, identify, locate, or register to, wounds, organs, and/or anatomical structures including a joint or an articular surface. In certain embodiments, a probe can be thin and/or pointed. In one embodiment, a probe is connected, integrated with, and/or coupled to another structure or instrument. In such an embodiment, the probe may serve to facilitate proper positioning of the another structure or instrument. For example, the probe may be used to identify and/or locate a particular anatomical structure and the positioning of the probe may then cause the connected structure or instrument to also be positioned in a desired location relative to one or more anatomical structures.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

“Friction fit” refers to a type of joint or connection that is created between two components by means of friction. A joint or connection that is formed using a friction fit may or may not include the use of additional fasteners such as screws, bolts, or adhesives. In a friction fit, the components are designed or configured to fit tightly together, creating enough friction between the surfaces to hold them securely in place, at least temporarily. The friction force is generated by the compressive force that is experienced between the components, and can be strong enough to prevent the components from separating under normal conditions. (© ChatGPT Mar. 23 Version, Modified, accessed chat.openai.com/chat May 2, 2023).

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.) As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

“Metatarsal” is a bone of a foot of a human or animal. In a human, a foot typically includes five metatarsals which are identified by number starting from the most medial metatarsal, which is referred to as a first metatarsal and moving laterally the next metatarsal is the second metatarsal, and the naming continues in like manner for the third, fourth, and fifth metatarsal. The metatarsal bone includes three parts a base which is a part that is at a proximal end of the metatarsal, a head which is a part that is at a distal end of the metatarsal, and a shaft or neck connects the base to the head.

“Epiphyses” refers to the rounded end of a long bone, at long bone's joint with adjacent bone(s). Between the epiphysis and diaphysis (the long midsection of the long bone) lies the metaphysis, including the epiphyseal plate (growth plate). At the joint, the epiphysis is covered with articular cartilage; below that covering is a zone similar to the epiphyseal plate, known as subchondral bone. (Search ‘epiphysis’ on Wikipedia.com 17 Jun. 2022. Modified. Accessed Aug. 1, 2022.) “Metaphysis” refers to the neck portion of a long bone between the epiphysis and the diaphysis. The metaphysis contains the growth plate, the part of the bone that grows during childhood, and as the metaphysis grows the metaphysis ossifies near the diaphysis and the epiphyses. (Search ‘metaphysis’ on Wikipedia.com 17 Jun. 2022. Modified. Accessed Aug. 1, 2022.) “Diaphysis” refers to the main or midsection (shaft) of a long bone. The diaphysis is made up of cortical bone and usually contains bone marrow and adipose tissue (fat). The diaphysis is a middle tubular part composed of compact bone which surrounds a central marrow cavity which contains red or yellow marrow. In diaphysis, primary ossification occurs. (Search ‘diaphysis’ on Wikipedia.com 17 Jun. 2022. Modified. Accessed Aug. 1, 2022.)

“Metaphyseal Diaphyseal Junction” or “MDJ” refers to an area of a long bone between the Metaphysis and the Diaphysis. This area can also include or be referred to as the epiphyseal plate (growth) plate. For certain surgical procedures, performing an osteotomy at or near the metaphyseal diaphyseal junction may be advantageous and desirable to promote rapid fusion of two cut faces formed in the osteotomy and bone growth to close the osteotomy, and/or may mitigate the risk of a nonunion of the osteotomy.

As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position. Certain bones such as a metatarsal bone can include a base as one structural component of the bone.

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

“Reduction guide” or “reducer” refers to any structure, apparatus, surface, device, system, feature, or aspect configured, designed, engineered, or fabricated to reduce or aide a user in the reduction of one bone or bone fragment or implant in relation to another bone or bone fragment or implant.

“Rotation guide” or “rotator” refers to any structure, apparatus, surface, device, system, feature, or aspect configured, designed, engineered, or fabricated to rotate or aid a user in the rotation of one structure relative to another structure. In certain embodiments, a rotation guide or rotator may be used to help a surgeon rotate one or more bones, parts of bones, bone fragment, an implant, or other anatomical structure, either alone or in relation to another one or more bones, parts of bones, bone fragments, implants, or other anatomical structures.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Metatarsal base resection guide” refers to a resection guide designed, engineered, fabricated, or intended for use with, one, in, or about a base part, section, surface, portion, or aspect of a metatarsal for one or more steps of a medical procedure. The metatarsal base resection guide may be used to form an osteotomy, to resect a wedge for a closing wedge procedure, resect a bone wedge that preserves a cortical layer of bone opposite the resected bone wedge, form an osteotomy that uniplanar wedge, a biplanar wedge, or a triplane wedge. Various embodiments of a metatarsal base resection guide may be used on a medial surface, a dorsal surface, a lateral surface, or a plantar surface of a single metatarsal. Alternatively, or in addition, various embodiments of a metatarsal base resection guide can be used on two or more metatarsals.

“Reduction guide” or “reducer” refers to any structure, apparatus, surface, device, system, feature, or aspect configured, designed, engineered, or fabricated to reduce or aide a user in the reduction of one bone or bone fragment or implant in relation to another bone or bone fragment or implant.

“Fastener guide” or “reducer” refers to any structure, apparatus, surface, device, system, feature, or aspect configured, designed, engineered, or fabricated to guide or direct a fastener into a bone as part of deploying the fastener. Examples of a fastener guide include an opening in a structure that is sized and/or oriented for deployment of a fastener such as a bone screw, a reference pin for aligning a fastener for deployment at a desired orientation and/or trajectory, and the like.

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers to the process of partitioning an image into different meaningful segments. These segments may correspond to different tissue classes, organs, pathologies, bones, or other biologically relevant structures. Medical image segmentation accommodates imaging ambiguities such as by low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities. Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer controls the position of the X-ray sources and detectors. Magnetic Resonance Imaging (MRI) is another medical imaging technology. Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.) Data, including images, text, and other data associated with medical imaging is referred to as patient imaging data. As used herein, “patient imaging data” refers to data identified, used, collected, gathered, and/or generated in connection with medical imaging and/or medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.) As used herein, “additive manufacturing” refers to a manufacturing process in which materials are joined together in a process that repeatedly builds one layer on top of another to generate a three-dimensional structure or object. Additive manufacturing may also be referred to using different terms including: additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

“Reference” refers to any apparatus, structure, device, system, component, marking, and/or indicator organized, configured, designed, engineered, and/or arranged to serve as a source of information or a point of comparison used to support or establish knowledge, truth, or quality. (© ChatGPT January 9 Version, Modified, accessed chat.openai.com/chat Jan. 28, 2023). In certain embodiments, a reference can serve as a starting point or initial position for one or more steps in a surgical procedure. A reference may be a type of fiducial. In certain embodiments, “reference” can be with a an adjective describing the reference. For example, a “model reference” is a reference within a model such as a computer model. A model reference refers to any feature, aspect, and/or component within a model. Examples of a model reference include, but are not limited to, a point, a plane, a line, a plurality of points, a surface, an anatomical structure, a shape, or the like. An “anatomical reference” is a reference within, on, near, or otherwise associated with an anatomical structure such as a bone. A reference (e.g., model, actual, virtual, and/or real) may also be referred to as a reference feature.

“Reference feature” refers to a feature configured for use as a point, plane, axis, or line of reference (aka a reference). A reference or reference feature can be used to position, measure, orient, fixation, couple, engage, and/or align one object or structure with another object or structure. In certain embodiments, a reference or reference feature can serve as a baseline, a ground truth, a waypoint, a control point, a landmark, and/or the like. A reference feature can facilitate moving from one coordinate system or frame of reference in a virtual environment to a position, location, frame of reference, environment, or orientation on, or in, an actual object, structure, device, apparatus, anatomical structure, or the like. Advantageously, a reference feature can coordinate objects, models, or structures in a digital or virtual model or representation with corresponding objects or structures (e.g., anatomical structures) of actual physical objects or structures. Said another way, a reference feature can serve to map from a virtual or modeled object to an actual or physical object. As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “alignment feature,” “securing feature,” “placement feature,” “protruding feature,” “engagement feature,” “disengagement feature,” “resection feature”, “guide feature”, “alignment feature,” and the like.

As used herein, a “marking” or “marker” refers to a symbol, letter, lettering, word, phrase, icon, design, color, diagram, indicator, figure, structure, device, apparatus, surface, component, system, or combination of these designed, intended, structured, organized, configured, programmed, arranged, or engineered to communication information and/or a message to a user receiving, viewing, or encountering the marking. The marking or “marker” can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

“Set” refers to a collection of objects. A set can have zero or more objects in the collection. Generally, a set includes one or more objects in the collection.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

“Fusion” refers to a natural process of bone growth and generation in which two separate bones and/or bone fragments grow together as new bone grows when the two separate bones and/or bone fragments contact each other. Often, fusion is facilitated by compression of the two separate bones and/or bone fragments towards each other.

As used herein, “image registration” refers to a method, process, module, component, apparatus, and/or system that seeks to achieve precision in the alignment of two images. As used here, “image” may refer to either or both an image of a structure or object and another image or a model (e.g., a computer based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed. (Search “medical image computing” on Wikipedia.com Jun. 24, 2021. CC-BY-SA 3.0 Modified. Accessed Jun. 25, 2021.)

“Register” or “Registration” refers to an act of aligning, mating, contacting, engaging, or coupling one or more parts and/or surfaces of one object in relation to one or more parts and/or surfaces of another object. Often, the one or more parts and/or surfaces of one object include protrusions and/or depressions that are the inverse or mirror configuration of protrusions and/or depressions of one or more parts and/or surfaces of the other object.

“Registration key” refers to a structure, surface, feature, module, component, apparatus, and/or system that facilitates, enables, guides, promotes, precision in the alignment of two objects by way of registration. In one aspect a registration key can include a surface and one or more recesses and/or features of that surface that are configured to fit within corresponding recesses, projections, and/or other features of another structure such as another surface. In one aspect a registration key can include a surface and one or more projections and/or features of, extending from, or connected to that surface that are configured to fit within corresponding recesses, projections, and/or other features of another structure such as another surface. In certain aspects, the features of the registration key may be configured to fit within, or in contact, or in close contact with those of the another structure. In one embodiment, when the two structures align the registration key has served its purpose.

As used herein, a “resection” refers to a method, procedure, or step that removes tissue from another anatomical structure or body. A resection can include an osteotomy that cuts through a bone or other tissue because the osteotomy still removes at least a minimal amount of tissue. A resection is typically performed by a surgeon on a part of a body of a patient. A resection is a type of osteotomy. (Search “surgery” on Wikipedia.com May 26, 2021. CC-BY-SA 3.0 Modified. Accessed May 26, 2021.) Resection may be used as a noun or a verb. In the verb form, the term is “resect” and refers to an act of performing, or doing, a resection. Past tense of the verb resect is resected.

“Anatomical structure” refers to any part or portion of a part of a body of a person, animal, or other patient. Examples of anatomical structures, include but are not limited to, a bone, bones, soft tissue, a joint, joints, a tissue surface, a protrusion, a recess, an opening, skin, hard tissue, teeth, mouth, eyes, hair, nails, fingers, toes, legs, arms, torso, vertebrae, ligaments, tendons, organs, or the like.

“Anatomical reference” refers to any reference(s) that is, or is on, or is in, or is otherwise associated, with an anatomical structure. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, a hole, a post, a plurality of holes, a plurality of posts, a fastener, a suture, a clamp, an instrument, an implant, or the like.

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, pain, discomfort, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “navigation guide” that guides a user in navigating a course or process or procedure such as a surgical procedure, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide.”

Those of skill in the art will appreciate that a resection feature may take a variety of forms and may include a single feature or one or more features that together form the resection feature. In certain embodiments, the resection feature may take the form of one or more slots or cut channels. Alternatively, or in addition, a resection feature may be referenced using other names including, but not limited to, channel, cut channels, and the like.

“Cross section” or “cross-section” refers to the non-empty intersection of a body in three-dimensional space with a plane, or the analog in higher-dimensional spaces. (Search “cross section” on Wikipedia.com Mar. 7, 2022. Modified. Accessed Sep. 21, 2022.)

“Cut channel” refers to a channel, slot, hole, or opening, configured to facilitate making a cut. In certain embodiments, a cut channel is one example of a resection feature, resection member, and/or resection guide. “Rotation slot” refers to a channel, slot, hole, or opening, configured to facilitate rotating one structure in relation to another structure.

As used herein, “slot” refers to a narrow opening or groove. (search “slot” on Merriam-Webster.com. Merriam-Webster, 2021. Web. 4 Aug. 2021. Modified.)

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

As used herein, an “interface,” “user interface,” or “engagement interface” refers to an area, a boundary, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

“Resection interface” refers to an interface between a resected portion of tissue and another object, structure, or thing. Often a resection interface is an interface or boundary between one resected portion of an anatomical structure and another resected portion of another anatomical structure. The two anatomical structures can be portions, parts, or fragments of one anatomical structure or two different anatomical structures. A resection interface can be embodied in a variety of shapes and/or configurations, including a point, a line, a plane, a contour, a boundary, or the like. In one embodiment, a resection interface is an interface between two or more cut planes or two or more cut surfaces or two or more cut faces.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Cortex” refers to an area of bone that extends from an external surface of the bone towards a center part of the bone. The cortex is typically comprised of cortical bone.

“Transosseous placement feature” refers to a placement feature that extends through one or more bones and that enables, or facilitates, placement of another device, apparatus, or instrument.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Prescription” or “Prescribed” refers to a written order, as by a physician or nurse practitioner, for the administration of a medicine, preparation of an implant, preparation of an instrument, or other intervention. Prescription can also refer to the prescribed medicine or intervention. (Search “prescription” on wordhippo.com. WordHippo, 2023. Web. Accessed 3 May 2023. Modified.)

As used herein, “end” refers to a part or structure of an area or span that lies at the boundary or edge. An end can also refer to a point that marks the extent of something and/or a point where something ceases to exist. An end can also refer to an extreme or last part lengthwise of a structure or surface. (search “end” on Merriam-Webster.com. Merriam-Webster, 2021. Web. 4 Aug. 2021. Modified.)

As used herein, “edge” refers to a structure, boundary, or line where an object, surface, or area begins or ends. An edge can also refer to a boundary or perimeter between two structures, objects, or surfaces. An edge can also refer to a narrow part adjacent to a border. (search “edge” on Merriam-Webster.com. Merriam-Webster, 2021. Web. 3 Aug. 2021. Modified.) In certain embodiments, an edge can be a one dimensional or a two dimensional structure that joins two adjacent structures or surfaces. Furthermore, an edge may be at a perimeter of an object or within a perimeter or boundary of an object.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, ankle joints, articulations of foot, and the like. (Search “joint” on Wikipedia.com Dec. 19, 2021. CC-BY-SA 3.0 Modified. Accessed Jan. 20, 2022.)

“Tarso-metatarsal joint” or “TMT joint” refers to a joint of a patient between a metatarsal bone and one or more cuneiform/tarsal/cuboid bones. The TMT joint may also be referred to as a “Lis Franc” or “Lisfranc” joint after a French surgeon Lisfranc.

“Cut surface” refers to a surface of an object that is created or formed by the removal of one or more parts of the object that includes the original surface. Cut surfaces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface can include manual, mechanical, motorized, hydraulic, automated, robotic, and the like. In certain embodiments, the cut surface(s) are planar.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” or “Long axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

“Mechanical axis” refers to an axis of a long bone such as a femur or tibia. The mechanical axis of a long bone is a straight line connecting the joint center points of the proximal and distal joint regions, whether in the frontal or sagittal plane. A mechanical axis can be useful in defining how the mechanical (weight, gait, flexion, extension, etc.) forces impact the morphology of the bone structure. A mechanical axis and anatomical axis can both help in the surgical planning in relation to deformed bones. (Search “axes of the long bones” on appropedia.com; Amit Dinanath Maurya, OpenSurgiSim (2021-2023). “Axes of the long bones—Mechanical and Anatomical”. SELF. Modified. Accessed Jun. 28, 2023.)

As used herein, a “drive”, “drive feature”, or “drive recess” refers to an apparatus, instrument, structure, member, device, component, system, or assembly structured, organized, configured, designed, arranged, or engineered to receive a torque and transfer that torque to a structure connected or coupled to the drive. At a minimum, a drive is a set of shaped cavities and/or protrusions on a structure that allows torque to be applied to the structure. Often, a drive includes a mating tool, known as a driver. For example, cavities and/or protrusions on a head of a screw are one kind of drive and an example of a corresponding mating tool is a screwdriver, that is used to turn the screw, the drive. Examples of a drive include but are not limited to screw drives such as slotted drives, cruciform drives, square drives, multiple square drives, internal polygon, internal hex drives, penta lobular sockets, hex lobular sockets, combination drives, external drives, tamper-resistant drives, and the like. (Search ‘list of screw drives’ on Wikipedia.com Mar. 12, 2021. Modified. Accessed Mar. 19, 2021.)

“Thread” or “threads” refers to a helical structure used to convert between rotational and linear movement or force. A thread is a ridge wrapped around a cylinder or cone in the form of a helix, with the ridge wrapped around the cylinder being called a straight thread and the ridge wrapped around the cone called a tapered thread. Straight threads or tapered threads are examples of external threads, also referred to as male threads. Threads that a correspond to male threads are referred to as female threads and are formed within the inside wall of a matching hole, passage, or opening of a nut or substrate or other structure. A thread used with a fastener may be referred to as a screw thread and can be an important feature of a simple machine and also as a threaded fastener. The mechanical advantage of a threaded fastener depends on its lead, which is the linear distance the threaded fastener travels in one revolution. (Search ‘screw thread’ on Wikipedia.com Jul. 17, 2022. Modified. Accessed Aug. 1, 2022.)

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

As used herein, a “shaft” refers to a long narrow structure, device, component, member, system, or assembly that is structured, organized, configured, designed, arranged, or engineered to support and/or connect a structure, device, component, member, system, connected to each end of the shaft. Typically, a shaft is configured to provide rigid support and integrity in view of a variety of forces including tensile force, compression force, torsion force, shear force, and the like. In addition, a shaft can be configured to provide rigid structural support and integrity in view of a loads including axial loads, torsional loads, transverse loads, and the like. A shaft may be oriented and function in a variety of orientations including vertical, horizontal, or any orientation between these and in two or three dimensions. A shaft may be made from a variety of materials including, but not limited to, metal, plastic, ceramic, wood, fiberglass, acrylic, carbon, biocompatible materials, biodegradable materials or the like. A shaft may be formed of any biocompatible materials, including but not limited to biocompatible metals such as Titanium, Titanium alloys, stainless steel, carbon fiber, combinations of carbon fiber and a metallic alloy, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others, or any combination of these materials.

“Head” refers to a device, apparatus, member, component, system, assembly, module, subsystem, circuit, or structure, organized, configured, designed, arranged, or engineered to have a prominent role in a particular feature, function, operation, process, method, and/or procedure for a device, apparatus, member, component, system, assembly, module, subsystem, circuit, or structure the includes, is coupled to, or interfaces with the head. In certain embodiments, the head may sit at the top or in another prominent position when interfacing with and/or coupled to a device, apparatus, member, component, system, assembly, module, subsystem, circuit, or structure.

As used herein, an “interface,” “user interface,” or “engagement interface” refers to an area, a boundary, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like.

The present disclosure discloses surgical systems and methods by which a bone condition, that can include a deformity, may be corrected or otherwise addressed. Known methods of addressing bone conditions are often limited to a finite range of discretely sized instruments. A patient with an unusual condition, or anatomy that falls between instrument sizes, may not be readily treated with such systems.

Furthermore, patient-specific instruments may be used for various other procedures on the foot, or on other bones of the musculoskeletal system. For example, patient-specific instruments and/or other instruments may be used for various procedures including resection and translation of a head of a long bone, determining where to perform an osteotomy on one or more joints or part of one or more bones, determining ligament or tendon attachment or anchoring points, determining where to form bone tunnels or position anchors, tendon or graft deployment, and the like.

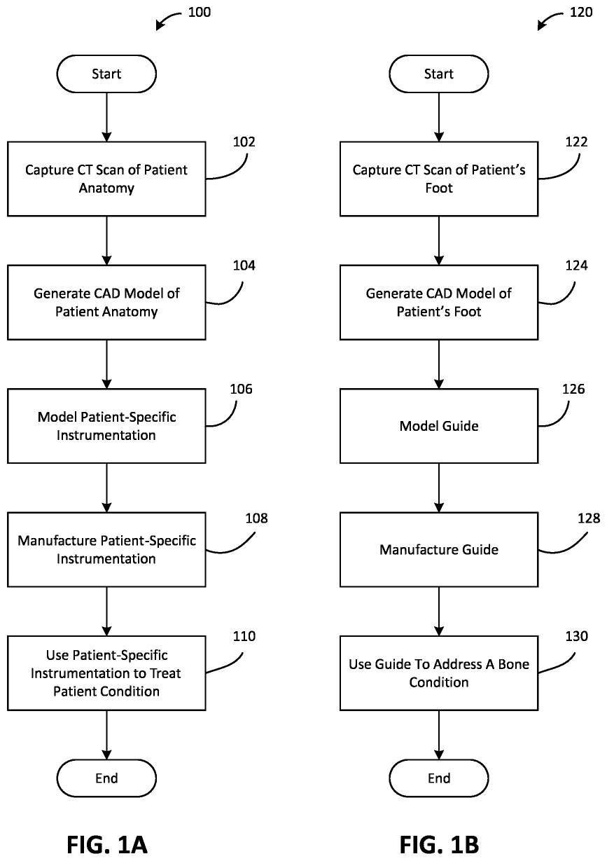

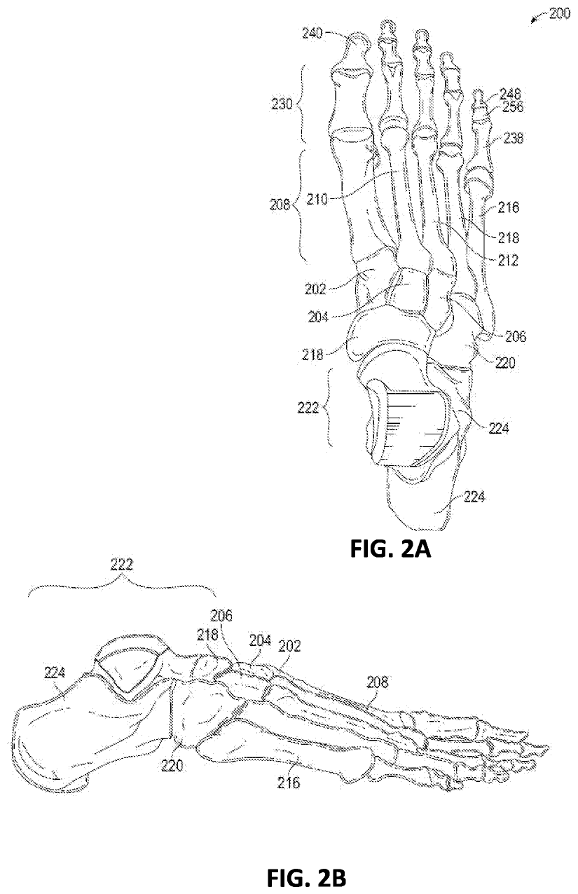

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may include capturing a scan of only the particular bone(s) to be treated, or may include capture of additional anatomic information, such as the surrounding tissues. Additionally or alternatively, the step 102 may include receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan, and generate one or more instruments that are matched specifically to the size and/or shape of the patient's bone(s). In some embodiments, such instrumentation may include a targeting guide, trajectory guide, drill guide, cutting guide, tendon trajectory guide, capital fragment positioning guide, or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioner, positioning guide, tendon trajectory guide, or the like.

In addition to, or in the alternative to the step 108, the model(s) may be used to select from available sizes of implants and/or instruments or instruments having various attributes and advise the surgeon accordingly. For example, where a range of guides are available for a given procedure, analysis of the CAD data may facilitate pre-operative selection of the optimal guide and/or optimal placement of the guide on the bone. Similarly, if a range of implants and/or instruments may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may include placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, an advanced computer analysis system, machine learning and automated/artificial intelligence may be used to generate a CAD model and/or one or more patient-specific instruments and/or operation plans. For example, a patient-specific instrument may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific instrument and the patient-specific synthetic cadaver can be provided to a surgeon who can then rehearse an operation procedure in part or in full before going into an operating room with the patient.

In certain embodiments, the patient-specific instrument or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific instrument and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, deformity, and/or tendon condition of a patient.

As shown, the method 120 may begin with a step 122 in which a CT scan (or another three-dimensional image) of the patient's foot is obtained. The step 122 may include capturing a scan of select bones of a patient or may include capturing additional anatomic information, such as the entire foot. Additionally or alternatively, the step 122 may include receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing an instrument with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured instrument may be used in surgery to facilitate treatment of the condition. In certain embodiments, a bone engagement surface of the instrument may be placed against the corresponding contours of the bone. The instrument may include an opening and/or trajectory guide to guide insertion of a trajectory guide such as a temporary fastener such as a K-wire. The instrument may then be removed, and the remaining steps of a surgical procedure performed.

Method 100 and method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

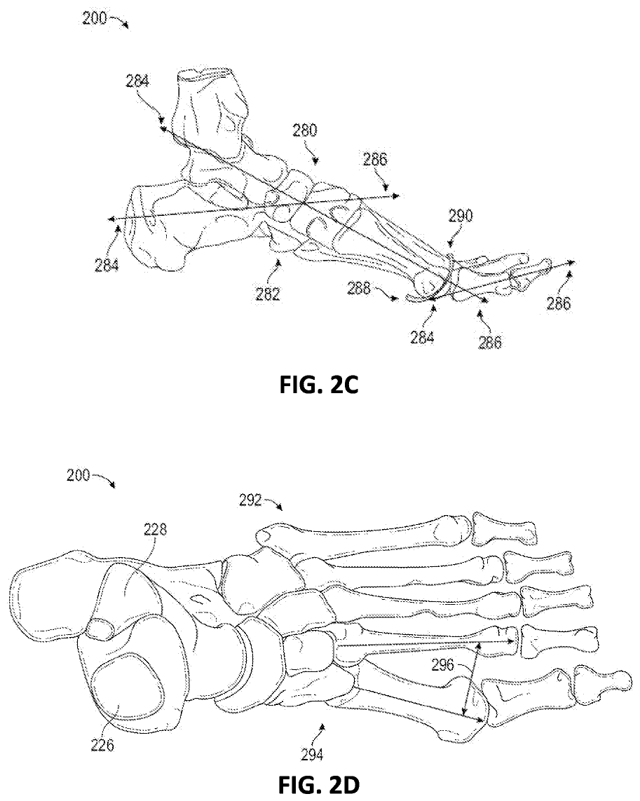

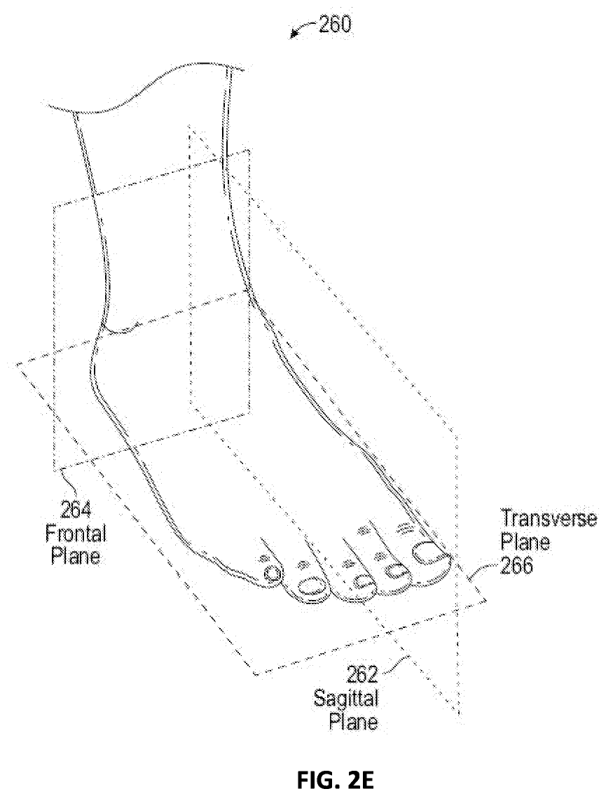

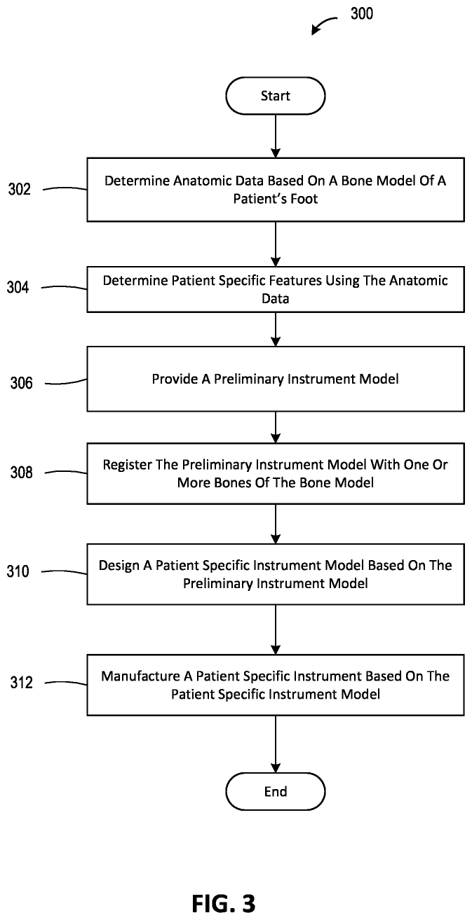

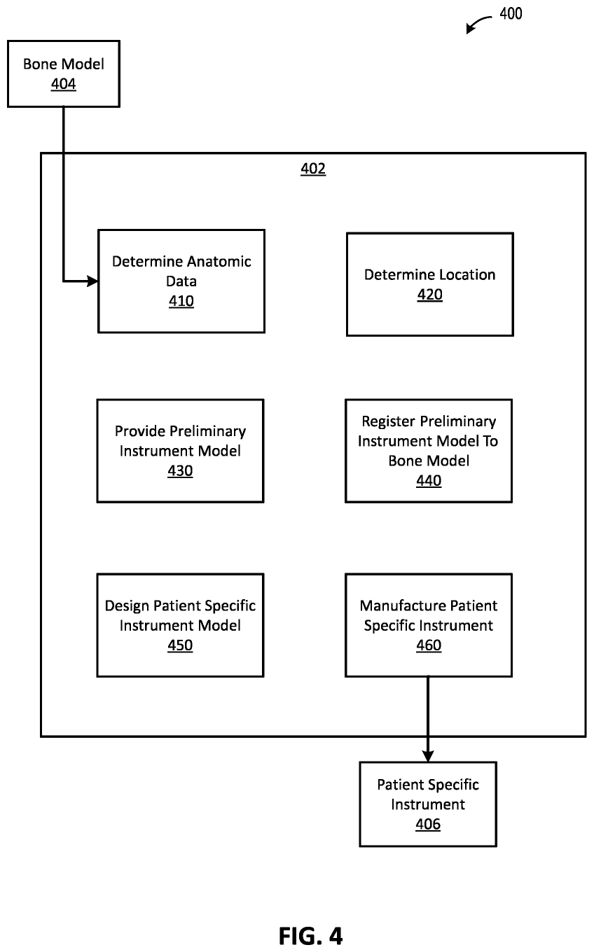

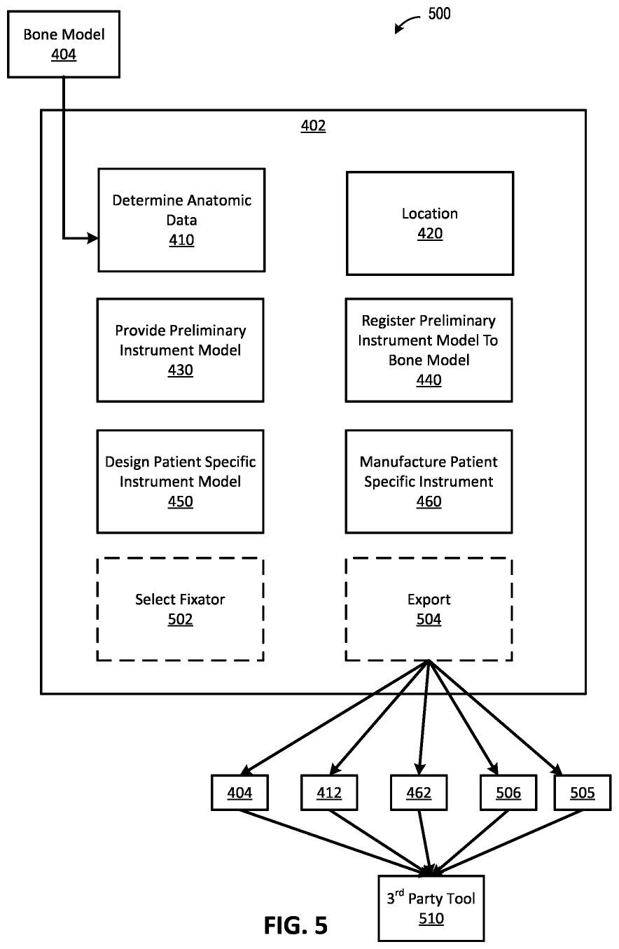

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, talus 222, and calcaneus 224, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. The foot 200, as illustrated, may have a tibia 226 and a fibula 228, among others. Dorsal refers to the top of the foot. Plantar refers to the bottom of the foot. Proximal 284 is defined as “closer to the primary attachment point”. Distal 286 is defined as “further away from the attachment point”. Plantar—flex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific instrument may help the surgeon obtain an optimal realignment, target, or position a bone tunnel, position one or more resections and/or fasteners and the like. Thus, providing patient-specific instruments, jigs, and/or instrumentation may provide unique benefits.

The present patient-specific instrumentation may be used to correct a wide variety of conditions. Such conditions include, but are not limited to, angular deformities of one bone in either the lower or upper extremities (for example, tibial deformities, calcaneal deformities, femoral deformities, and radial deformities). The present disclosure may also be used to treat an interface between two bones (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

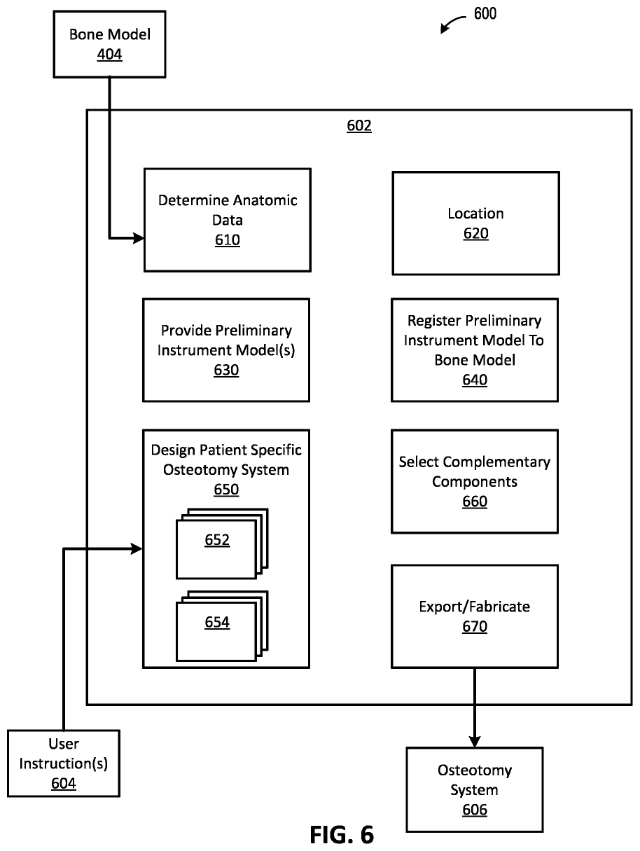

FIG. 3 illustrates a flowchart diagram depicting a method 300 for generating one or more instruments (which may or may not be patient-specific) configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 one or more angles (e.g., trajectory angle) and/or patient-specific features for a procedure using the anatomic data. “Trajectory angle” refers to a recommended angle for deployment of an instrument, graft, body part, or resection feature angle relative to a bone of a patient for a procedure. In certain embodiments, determining steps, instruments, and/or implants for a corrective procedure may employ an advanced computer analysis system, expert systems, machine learning, and/or automated/artificial intelligence.

Next, the method 300 may proceed and a preliminary instrument model is provided 306 from a repository of template models. A preliminary instrument model is a model of a preliminary instrument.

As used herein, “preliminary instrument” refers to an instrument configured, designed, and/or engineered to serve as a template, prototype, archetype, or starting point for creating, generating, or fabricating a patient-specific instrument. In one aspect, the preliminary instrument may be used, as-is, without any further changes, modifications, or adjustments and thus become a patient-specific instrument. In another aspect, the preliminary instrument may be modified, adjusted, or configured to more specifically address the goals, objectives, or needs of a patient or a surgeon and by way of the modifications become a patient-specific instrument. The patient-specific instrument can be used by a user, such as a surgeon, to guide steps in a surgical procedure, such as an osteotomy, graft harvest (e.g., autograft, allograft, or xenograft), minimally invasive surgical (MIS) procedure, and/or a tendon transfer procedure. Accordingly, a preliminary instrument model can be used to generate a patient-specific instrument. The patient-specific instrument model may be used in a surgical procedure to facilitate one or more steps of the procedure, and may be used to generate a patient-specific instrument that can be used in a surgical procedure for the patient.

In certain embodiments, the preliminary instrument model may be generated based on anatomic data and/or a bone model or a combination of these, and no model or predesigned structure, template, or prototype. Alternatively, or in addition, the preliminary instrument model may be, or may originate from, a template instrument model selected from a set of template instrument models. Each model in the set of template instrument models may be configured to fit an average patient's foot. The template instrument model may subsequently be modified or revised by an automated process or manual process to generate the preliminary instrument model used in this disclosure.

As used herein, “template instrument” refers to an instrument configured, designed, and/or engineered to serve as a template for creating, generating, or fabricating a patient-specific instrument. In one aspect, the template instrument may be used, as-is, without any further changes, modifications, or adjustments and thus become a patient-specific instrument. In another aspect, the template instrument may be modified, adjusted, or configured to more specifically address the goals, objectives, or needs of a patient or a surgeon and by way of the modifications become a patient-specific instrument. The patient-specific instrument can be used by a user, such as a surgeon, to guide making one or more resections of a structure, such as a bone for a procedure. Accordingly, a template instrument model can be used to generate a patient-specific instrument model. The patient-specific instrument model may be used in a surgical procedure to address, correct, or mitigate effects of the identified deformity and may be used to generate a patient-specific instrument that can be used in a surgical procedure for the patient.

Next, the method 300 may register 308 the preliminary instrument model with one or more bones of the bone model. This step 308 facilitates customization and modification of the preliminary instrument model to generate a patient-specific instrument model from which a patient-specific instrument can be generated. The registration step 308 may combine two models and/or patient imaging data and position both models for use in one system and/or in one model.

Next, the method 300 may design 310 a patient-specific instrument and/or procedure model based on the preliminary instrument model. The design step 310 may be completely automated or may optionally permit a user to make changes to a preliminary instrument model or partially completed patient-specific instrument model before the patient-specific instrument model is complete. A preliminary instrument model and patient-specific instrument model are two examples of an instrument model. As used herein, “instrument model” refers to a model, either physical or digital, that represents an instrument, tool, apparatus, or device. Examples, of an instrument model can include a cutting instrument model, a resection instrument model, an alignment instrument model, a reduction instrument model, a patient-specific tendon trajectory instrument model, graft harvesting instrument model, minimally invasive surgical (MIS) positioner model, or the like. In one embodiment, a patient-specific instrument and a patient-specific instrument model may be unique to a particular patient and that patient's anatomy and/or condition.

The method 300 may conclude by a step 312 in which a patient-specific instrument may be manufactured based on the patient-specific instrument model. Various manufacturing tools, devices, systems, and/or techniques can be used to manufacture the patient-specific instrument.

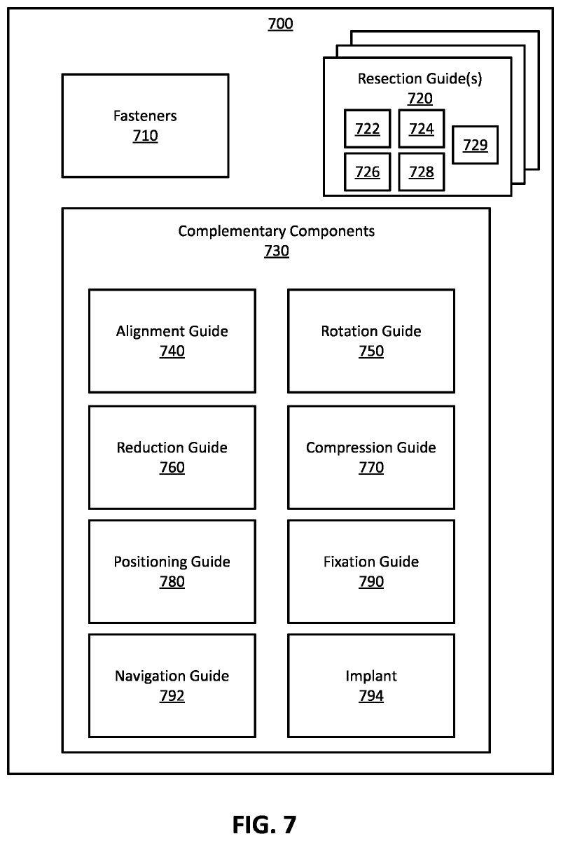

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific instrument 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one or more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one or more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, X-ray, and bone density scans can be combined to effectively create an anatomic map that determines the structural integrity of the underlying bone.

Identifying the structural integrity of the underlying bone can help in determining where bone resections (e.g., osteotomies) can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument and/or soft tissue based on the anatomic data 412 and/or the bone model 404. In one embodiment, the location module 420 may compare the anatomic data 412 to a general model that is representative of most patient's anatomies and may be free from deformities or anomalies. The location module 420 can operate autonomously and/or may facilitate input and/or revisions from a user. The location module 420 may be completely automated, partially automated, or completely manual. A user may control how automated or manual the determining of the location and/or trajectory angles is.

The provision module 430 is configured to provide a preliminary instrument model 438. The provision module 430 may use a variety of methods to provide the preliminary instrument model. In one embodiment, the provision module 430 may generate a preliminary instrument model. In the same, or an alternative embodiment, the provision module 430 may select a template instrument model for a tendon (or tendon substitute) deployment procedure configured to enable locating the position and/or providing the trajectory provided by the location module 420. In one embodiment, the provision module 430 may select a template instrument model for a minimally invasive surgical (MIS) bunion correction procedure configured to enable locating the position and/or providing the trajectory for the fixation deployment. In one embodiment, the provision module 430 may select a template instrument model from a set of template instrument models (e.g., a library, set, or repository of template instrument models).

The registration module 440 registers the preliminary instrument model with one or more bones or other anatomical structures of the bone model 404. As explained above, registration is a process of combining medical imaging data, patient imaging data, and/or one or more models such that the preliminary instrument model can be used with the bone model 404.

The design module 450 designs a patient-specific instrument (or patient-specific instrument model) based on the preliminary instrument model. The design operation of the design module 450 may be completely automated, partially automated, or completely manual. A user may control how automated or manual the designing of the patient-specific instrument (or patient-specific instrument model) is.

The manufacturing module 460 may manufacture a patient-specific instrument 406 using the preliminary instrument model. The manufacturing module 460 may use a patient-specific instrument model generated from the preliminary instrument model. The manufacturing module 460 may provide the patient-specific instrument model to one or more manufacturing tools and/or fabrication tool (e.g., additive and/or subtractive). The patient-specific instrument model may be sent to the tools in any format such as an STL file or any other CAD modeling or CAM file or method for data exchange. In one embodiment, a user can adjust default parameters for the patient-specific instrument such as types and/or thicknesses of materials, dimensions, and the like before the manufacturing module 460 provides the patient-specific instrument model to a manufacturing tool.

Effective connection of the guide to one or more bones can ensure that surgical steps are performed in desired locations and/or with desired orientations and mitigate undesired surgical outcomes.

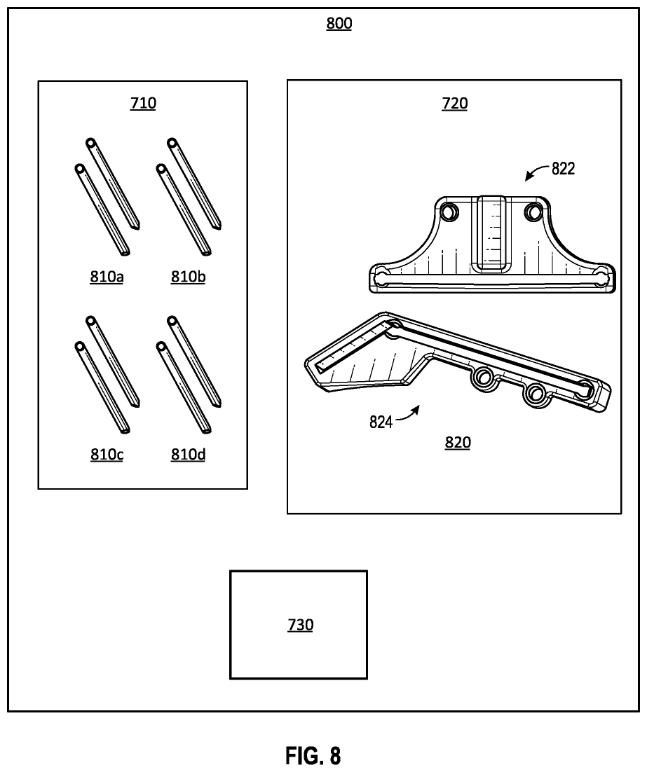

FIG. 5 illustrates an exemplary system 500 configured to generate one or more patient-specific instruments configured to correct a bone condition, according to one embodiment. The system 500 may include similar components or modules to those described in relation to FIG. 4. In addition, the system 500 may include a fixator selector 502 and/or an export module 504.

The fixator selector 502 enables a user to determine which fixator(s) to use for a MIS bunion correction procedure planned for a patient. In one embodiment, the fixator selector 502 may recommend one or more fixators based on the bone model 404, the location, the trajectory, or input from a user or a history of prior MIS bunion correction procedures performed. The fixator selector 502 may select a fixator model from a set of predefined fixator models or select a physical fixator from a set of fixators. The fixators may include a plate and associated accessories such as screws, anchors, and the like.

In one embodiment, the fixator selector 502 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that the artificial intelligence or machine learning module is able to select or recommend suitable fixator(s).

The export module 504 is configured to enable exporting of a patient-specific instrument model 462 for a variety of purposes including, but not limited to, fabrication/manufacture of a patient-specific instrument 406 and/or fixator(s), generation of a preoperative plan, generation of a physical bone model matching the bone model 404, and the like. In one embodiment, the export module 504 is configured to export the bone model 404, anatomic data 412, a patient-specific instrument model 462, a preoperative plan 506, a fixator model 508, or the like. In this manner the custom instrumentation and/or procedural steps for a procedure (e.g., a graft harvesting procedure, minimally invasive surgical (MIS) procedure, or the like) can be used in other tools. The preoperative plan 506 may include a set of step by step instructions or recommendations for a surgeon or other staff in performing a procedure (e.g., a graft harvesting procedure, minimally invasive surgical (MIS) procedure, or the like). The preoperative plan 506 may include images and text instructions and may include identification of instrumentation to be used for different steps of the procedure (e.g., a graft harvesting procedure, minimally invasive surgical (MIS) procedure, or the like). The instrumentation may include the patient-specific instrument 406 and/or one or more fixators/fasteners. In one embodiment, the export module 504 may provide a fixator model which can be used to fabricate a fixator for the procedure.

The exports (404, 412, 462, 506, and 508) may be inputs for a variety of 3rd party tools 510 including a manufacturing tool, a simulation tool, a virtual reality tool, an augmented reality tool, an operative procedure simulation tool, a robotic assistance tool, and the like. A surgeon can then use these tools when performing a procedure or for rehearsals and preparation for the procedure. For example, a physical model of the bones, patient-specific instrument 406, and/or fixators can be fabricated, and these can be used for a rehearsal operative procedure. Alternatively, a surgeon can use the bone model 404, preliminary instrument model 438, and/or a fixator model to perform a simulated procedure using an operative procedure simulation tool.

Referring now to FIGS. 3-5, certain methods, systems, and/or apparatuses are disclosed herein for preparing for, planning, outlining, and/or instrumenting, one or more surgical procedures. Alternatively, or in addition, the methods, systems, and/or apparatuses a disclosed herein can be used for preoperative development and design of systems, instrumentation, and/or implants and/or for preoperative rehearsal and/or instruction of a surgeon before the surgical procedure. For example, a surgeon can use the method 300, bone model(s) 404, patient instrument(s) 406, system 400, and/or apparatus 402 to perform a mock surgical procedure virtually before an actual surgical procedure.

These techniques and/or technologies can greatly advance the medical field and provide valuable instruction and experience to a surgeon prior to an actual surgical procedure. Furthermore, these techniques and/or technologies are made effective owing to the accuracy and precision of the models because of the fidelity of the medical imaging of the patient anatomy. This virtual modeling of patient anatomy has become very accurate and helpful, particularly for hard tissue such as bones and the surfaces of these bones.

Unfortunately, the fidelity and accuracy of these models is not as advanced with respect to the modeling of soft tissue of a patient such as sinews, skin, tendons, ligaments, muscles, fat, and the like. Thus, rehearsal of a surgical procedure, particularly one that includes translating and/or reorienting one or more bone fragments may have limited benefits. In such cases, because the surgeon cannot predict or know beforehand how much movement and reorientation the soft tissue of a patient will permit, the surgeon may need to revise or adapt a surgical procedure intraoperatively to achieve optimal outcomes. The system, apparatus, and methods of the present disclosure enable a surgeon to make intraoperative adjustments to surgical plan based on what the surgeon learns during the surgery.

The present disclosure leverages the use of models, such as computer models, and particularly models of a specific patient to provide and/or generate instrumentation, implants, and/or surgical plans that advanced patient care. Advantageously, these models are unique and customized for a particular patient. Thus, the models reflect the actual anatomical features and aspects of the patient.

However, the utility and helpfulness of the models, methods, systems, and/or apparatuses of FIGS. 3-5, may be dependent on how effectively a surgeon can navigate within, on, or in relation to one or more anatomical references or anatomical features of a patient such that the steps of the surgical procedure can be performed on a patient in the same manner as those modeled using models of the anatomy of the patient. This process of navigation is referred to as a mapping or translation between the virtual or model environment to a physical or real world environment that includes the patient anatomy and the operating field.

Advantageously, the models, methods, systems, and/or apparatuses of the present disclosure facilitate mapping or translating between a virtual or model environment and/or instrumentation to a physical or real world environment for a surgical procedure. The present disclosure provides this feature or benefit by providing an apparatus, system, and method, that enables a surgeon to identify, create, form, and/or use reference features for a surgical procedure. The reference feature provides a reference and/or starting point on, in, or associated with anatomy of a patient such that steps, stages, features, or aspects planned and configured within the model can be accurately performed on, with, or to the anatomy of the patient. In certain embodiments, one or more steps of a surgical procedure can be done in connection with or in relation to the reference feature.

The reference feature facilitates moving from one coordinate system or frame of reference in a virtual environment to a position, location, frame of reference, environment, or orientation on, or in, an actual object, structure, device, apparatus, anatomical structure, or the like. Advantageously, the reference feature can coordinate objects, models, or structures in a digital or virtual model or representation with corresponding objects or structures (e.g., anatomical structures) of actual physical objects or structures. Said another way, the reference feature can serve to map from a virtual or modeled object to an actual or physical object.

Advantageously, the embodiment of the present disclosure include features and aspects that assist a surgeon in locating at least one reference feature, which can then be used in one or more stages of a surgical procedure. In certain embodiments, the actual instruments fabricated using the present disclosure may include one or more references (e.g., a model references). The one or more model instruments may use the one or more references to position and/or orient the one or more model instruments such that other steps of a surgical procedure can be performed in relation to those one or more model instruments and/or model references. Certain model references may key off or related to anatomical references of modeled anatomical body parts. The reference feature(s) correspond to the model references and together enable a surgeon to identify reference features on actual anatomy of a patient for a surgical procedure.

In certain embodiments, one or more fasteners deployed in an instrument such as a resection guide can serve as reference features, for an initial stage of the surgical procedure and/or for subsequent stages of the surgical procedure. In certain embodiments, a bone engagement feature can serve as a reference feature for an osteotomy system and/or surgical procedure.

Advantageously, the embodiments of the present disclosure leverage patient-specific models of patient anatomy and the use of these models to generate patient-specific instruments as well as input from users of the osteotomy (e.g., surgeons). In one embodiment, this input is provided in the form of user directions. Combining patient-specific medical imaging, patient-specific anatomical models, and user directions enable the present disclosure to provide a customized or patient-specific osteotomy that serves the patient's needs as well as aides the surgeon in performing the surgical procedure. In this manner, a surgeon can perform the surgical procedure with higher confidence and assurance that the procedure performed on the patient will coincide with the plan set forth using models in a virtual environment. Consequently, the present disclosure improves the level of patient care and positive outcomes.

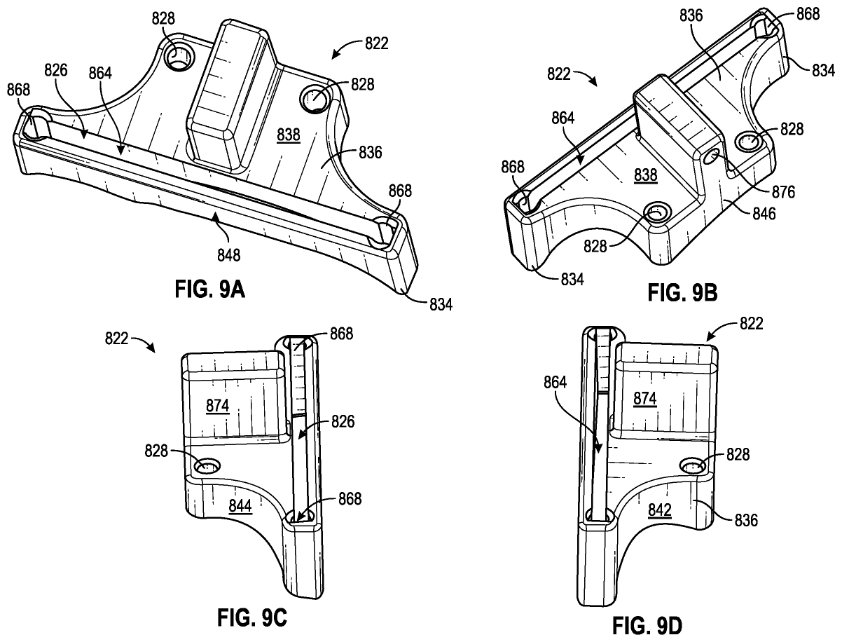

FIG. 6 illustrates an exemplary system 600 configured to design, generate, develop, and/or produce an osteotomy system, according to one embodiment. In certain embodiments, the osteotomy system can be patient-specific. One advantage of the present disclosed embodiments is that an end user of an osteotomy system (e.g., instruments, preoperative plan, implants, etc.) can have as much, or as little control or input, over one or more or all of the aspects of the osteotomy system. Furthermore, this osteotomy system can be customized both to the needs and specific aspects of the patient as well as to the needs and/or preferences and/or desires of the user (e.g., surgeon).

The system 600 may include similar components or modules to those described in relation to FIG. 4. The structures, features, and functions, operations, and configurations of the system 600 may be similar or identical to components or modules of system 400, like parts identified with similar reference numerals. Accordingly, the system 600 may include an apparatus 602 configured to accept, review, receive or reference a bone model 404 and user instructions 604 and provide a patient-specific osteotomy system 606. In one embodiment, the apparatus 602 is a computing device. In another embodiment, the apparatus 602 may be a combination of computing devices, systems, apparatuses, software components, single software component such as a software application, one or more third party manufacturers, or the like.

The apparatus 602 may include a determination module 610, a location module 620, a provision module 630, an optional registration module 640, a design module 650, a selection module 660, and an export/fabrication module 670. Each of which may be implemented in one or more of, software, hardware, or a combination of hardware and software. In certain embodiments, one or more parts of the system 600 may be operated by a user (e.g., a technician), a plurality of users, and may include input, involvement, and/or feedback from an end user of the osteotomy system developed. Generally, the end user of the osteotomy system will be a surgeon. Those of skill in the art will appreciate that depending on the surgical procedure being performed, one or more of the modules of the apparatus 602 may or may not be used.

The determination module 610 may operate in a similar manner to the determination module 410. The location module 620 may operate in a similar manner to the location module 420. The provision module 630 may operate in a similar manner to the provision module 430. The registration module 640 may operate in a similar manner to the registration module 440.

The design module 650 enables one or more users to design an osteotomy system 606 and in particular a patient-specific osteotomy system 606. A patient-specific osteotomy system 606 can include a number of different instruments, components, and/or systems, including but not limited to one or more cutting tools, one or more resection guides, one or more provisional fasteners, one or more fixation systems and/or instruments, a preoperative plan, one or more kits of implants and/or trial components, one or more alignment guides, one or more positioning guides, one or more reduction guides, one or more, one or more navigation guides, one or more fixation guides, one or more, one or more compression guides, one or more rotation guides, and the like. In addition, one or more of these components can be patient-specific. For example, the patient-specific osteotomy system 606 can include a patient-specific instrument, patient-specific trajectory guide, a patient-specific resection guide, a patient-specific cutting guide, a patient-specific positioning guide or positioner, another patient-specific instrument, or the like.

Alternatively, or in addition, the patient-specific osteotomy system 606 can include one or more subparts or components of each of the instruments, components and/or systems of the patient-specific osteotomy system 606. For example, in one embodiment, the design module 650 may enable a user and/or end user to determine and/or define a number, size, shape, position, orientation, trajectory and/or configuration for one or more bone attachment features, a number, size, shape, position, orientation, trajectory and/or configuration for one or more resection features, a number, size, shape, position, orientation, trajectory and/or configuration for one or more bone engagement features, a number, size, shape, position, orientation, trajectory and/or configuration for one or more bone engagement surfaces, a number, size, shape, position, orientation, trajectory and/or configuration for one or more fixators (either or both provisional or permanent), and the like.

Those of skill in the art will appreciate that the design module 650 offers a large variety of different options and combinations for the constituents of the patient-specific osteotomy system 606 as well as a plurality of options for the components of the patient-specific osteotomy system 606 and that such options may be overwhelming. Advantageously, the surgical procedure to be performed, the bone model 404, and user instructions 604 each alone and/or in combination define an initial set of members for the patient-specific osteotomy system 606. For example, certain well known surgical techniques have specific names and surgeons understand and/or have experience doing these procedures and therefore know what instruments will be needed for the surgical procedure.

In addition, each surgeon is different just as each patient is different. Therefore, surgeon experience and/or preferences may factor into the members of the patient-specific osteotomy system 606 a particular surgeon wants and/or the configuration of the members of the patient-specific osteotomy system 606. For example, where one surgeon may prefer to use two resection guides another surgeon may want to use one resection guide and perform other osteotomies for the surgical procedure manually or free-hand.

Based on the surgical procedure to be performed, many decisions about the design and/or make up of the patient-specific osteotomy system 606 can be made as recommendations and/or proposals by a technician to a surgeon. These decisions can be based in whole or in part on the surgical procedure to be performed, the bone model 404 and/or the user instructions 604.

For example, suppose a surgeon would like a patient-specific osteotomy system 606 for an ankle fusion procedure. One goal of the ankle fusion procedure may be to relieve pain of the patient and to remove a minimal amount of bone in the process of completing the procedure. In such an example, the bone model 404 may be of one or more bones of a foot and/or ankle of the patient. The surgeon may provide a request and/or a set of user instructions 604 for a patient-specific osteotomy system 606 for this ankle fusion procedure.

Those of skill in the art will appreciate that the user instructions 604 may be of a variety of different types, lengths, number of details and may be provided in a variety of different formats including oral, written, or the like. In one embodiment, the user instructions 604 may be a request for a patient-specific osteotomy system 606 that includes a set of default instruments, preoperative plan, implants, or the like. For example, the user instructions 604 may as short and simple as “Please provide an osteotomy system for an ankle fusion of the left ankle for patient <<identifying information (e.g., name, dob, etc.)>> with an anterior approach.” The user instructions 604 may be provided in the form of a product order, a purchase order, a prescription, or the like. The user instructions 604 may be provided in written manual/analog form, include a manual signature, digital form, include an e-signature, or the like. In addition, the user instructions 604 may include security and/or authorization features that enable the receiver to confirm that the user instructions 604 are valid and are authorized by a particular surgeon or doctor. The user instructions 604 may indicate the approach to the surgical site (e.g., an ankle or foot joint or bone) the surgeon wants to take, anterior, posterior, medial, lateral, or the like.

In another embodiment, the user instructions 604 may include specific instructions for the number and/or kind or type of components in the patient-specific osteotomy system 606 and/or the configuration of one or more of these components. For example, the user instructions 604 may identify a specific fixation product or fixation system the surgeon will be using for permanent fixation of the osteotom(ies). Alternatively, or in addition, the user instructions 604 may include designation of one or more complementary components and/or configurations for these components to be included in the patient-specific osteotomy system 606.

In one embodiment, the user instructions 604 may designate a particular material and/or mass for fabricating one or more guides to be included in the patient-specific osteotomy system 606. Some surgeons may find that patient-specific instruments, such as a patient-specific resection guide may more readily register to one or more bone surfaces if the instrument has a greater mass and/or weight. With the greater mass and a sufficient fidelity bone engagement surface, a patient-specific instrument may seem to find its own way or seek out a desired position on a bone that matches or substantially matches a position planned when the patient-specific osteotomy system 606 was developed. Consequently, a surgeon may request in the user instructions 604 that the instrument be made from a metal such as titanium.

With the bone model 404 and user instructions 604 a user such as a technician may operate the design module 650 alone or together with other modules of the apparatus 602 to develop a patient-specific osteotomy system 606. In certain embodiments, a single user operates the apparatus 602. Alternatively, or in addition, a plurality of users, which may include an end user, such as surgeon can operate or interact with one or more modules of the apparatus 602 as the patient-specific osteotomy system 606 is designed or developed.

In one embodiment, a technician may provide a patient-specific osteotomy system 606 that includes one or more complementary components and one or more resection guides, which may be patient-specific. The technician may also provide a preoperative plan. These may be provided to an end user (e.g., surgeon) either directly or by accessing the apparatus 602 remotely. The end user may review the preoperative plan and/or the components of the patient-specific osteotomy system 606 (e.g., resection guides) and may approve of the patient-specific osteotomy system 606 or may request changes. In certain embodiments, these changes may include the addition of one or more added bone engagement features, one or more resection guides, a change in a trajectory for a bone attachment feature, a change in trajectory for an osteotomy, an addition of openings in a guide to coincide with openings needed for a fixation system, as well as a plurality of other possible changes to the patient-specific osteotomy system 606. The technician may then make the requested changes and present a revised patient-specific osteotomy system 606 for the surgeon to review again. Next, the surgeon may approve of the revised patient-specific osteotomy system 606 and/or request additional changes.

In the illustrated embodiment, the design module 650 may include a plurality of resection features 652 and/or a plurality of bone engagement features 654. A technician may select one or more resection features 652 and/or one or more bone engagement features 654 and include them in the patient-specific osteotomy system 606. Alternatively, or in addition, a surgeon may designate which resection features 652 and/or bone engagement features 654 to include in the patient-specific osteotomy system 606.

In certain embodiments, the surgeon and technician may collaborate and/or consult with each other regarding the design and/or configuration of the patient-specific osteotomy system 606 and its components. The technician may share with the surgeon information about the technological features and/or limitations of the components of the patient-specific osteotomy system 606 and use the technician's experience and know-how to make recommendations to the surgeon. The surgeon can present ideas and/or requests regarding what the surgeon would like for components of the patient-specific osteotomy system 606 and the technician can determine whether those ideas/requests can be satisfied using a patient-specific osteotomy system 606.

The apparatus 602 uses both the bone model 404 and user instructions 604 to provide a patient-specific osteotomy system 606. Advantageously, the apparatus 602 enables a surgeon to be involved in the design and development of a patient-specific osteotomy system 606 that is suited not just for the patient, but also for the needs, skills and/or preferences of the surgeon. In this manner, a patient-specific osteotomy system 606 can be provided that improves patient care and accomplishing of desired outcomes.

In one embodiment, the operation of the design module 650 may be completely automated, partially automated, or completely manual. A user may control how automated or manual the designing of the patient-specific osteotomy system 606 is, including patient-specific instrument models, patient-specific instruments, and/or other components of the patient-specific osteotomy system 606.

The apparatus 602 may include a selection module 660 and an export/fabrication module 670. The selection module 660 facilitates the selection and/or customization of one or more complementary components for a patient-specific osteotomy system 606. Complementary components are described herein, but can include certain guides or other aids to facilitate completing a surgical procedure as planned. In one embodiment, the operation of the selection module 660 may be completely automated, partially automated, or completely manual. A user may control how automated or manual the selection module 660 is.

The export/fabrication module 670 is configured to enable exporting of a patient-specific osteotomy system 606 for a variety of purposes including, but not limited to, fabrication/manufacture of one or more patient-specific instruments and/or fixator(s), ordering or fabricating one or more members of the patient-specific osteotomy system 606, generation of a preoperative plan, generation of a physical bone model matching the bone model 404, and the like.

In one embodiment, the export/fabrication module 670 is configured to export the bone model 404, anatomic data 412, one or more patient-specific instrument models 462, a preoperative plan 506, a fixator model 508, or the like. In this manner the custom instrumentation and/or procedural steps for a procedure can be used in other tools. The preoperative plan 506 may include a set of step by step instructions or recommendations for a surgeon or other staff in performing a procedure (e.g., a graft harvesting procedure, minimally invasive surgical (MIS) procedure, or the like). The preoperative plan 506 may include images and text instructions and may include identification of instrumentation to be used for different steps of the procedure (e.g., a graft harvesting procedure, minimally invasive surgical (MIS) procedure, or the like). The instrumentation may include a patient-specific instrument, bone engagement features, and/or one or more fixators/fasteners.

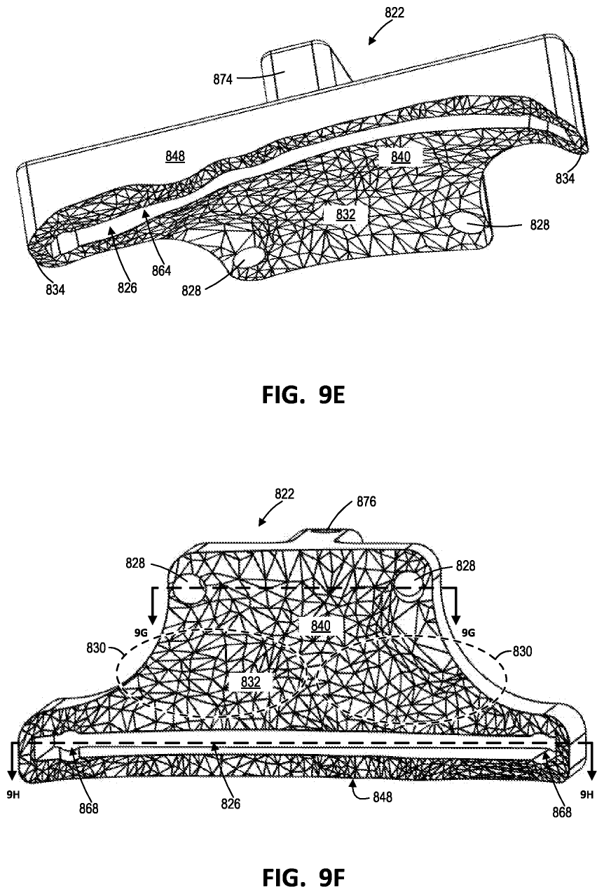

FIG. 7 illustrates an exemplary system 700 for remediating a condition present in a patient's foot, according to one embodiment. The system 700 can include one or more fasteners 710, one or more resection guides 720, and one or more complementary components 730. While a system 700 can be used for a variety of procedures, one or more features, components, and/or aspects of the system 700 may be particularly suited for one or more osteotomies on one or more bones of a structure such as a patient's foot, ankle, wrist, hand, shoulder, or the like.

In certain embodiments, the one or more fasteners 710 can include one or more permanent fasteners and/or one or more temporary fasteners. Typically, the fasteners 710 may be used during a variety of different steps of a procedure. Temporary fasteners are often used because they can securely hold bone or parts/fragments of bones while steps of the procedure are conducted. A common temporary fastener that can be used with system 700 is a K-wire, also referred to as a pin, guide pin, and/or anchor pin. Permanent fasteners 710 such as bone screws, bone staples, sutures, tethers or the like may also be used in a surgical procedure.

The one or more resection guides 720 assist a surgeon in performing different resection or dissection steps for an osteotomy or other procedure. In certain embodiments, a resection guide 720 includes one or more resection features 722 and one or more bone attachment features 724. The resection features 722 can take a variety of forms and/or embodiments. In one embodiment, the resection features 722 take the form of a cut channel or slot or other opening.

The resection features 722 provide a guide for a surgeon using a cutting tool to resect a bone, one or more bones, or other tissues of a patient. In certain embodiments, the resection features 722 may guide a surgeon in performing a resection, and osteotomy, and/or a dissection.

Similarly, the bone attachment features 724 can take a variety of forms and/or embodiments. The bone attachment features 724 may serve to secure the resection guide 720 and/or other instrumentation to one or more bones and/or one or more other structures. Often, a bone attachment feature 724 can take the form of a hole in and/or through the resection guide 720 together with a temporary fastener such as a K-wire, pin, or guide pin.

The bone attachment features 724 facilitate attachment (at least temporarily) of a resection guide 720 to one or more bones, or bone fragments, of a patient. The bone attachment features 724 may include any of a wide variety of fasteners or structures including, but not limited to, holes, spikes, prongs, screws, fastening devices, and/or the like. Effective connection of the resection guide 720 to one or more bones across a joint and/or to one or more bones can ensure that cut surfaces are formed in desired locations and orientations and mitigate removal of hard tissue and/or soft tissue in undesired locations and/or orientations.

In certain embodiments, a resection guide 720 may include one or more bone engagement surfaces 726 and/or one or more landmark registration features 728. In certain embodiments, a landmark registration feature 728 may extend from one or more sides or ends of a resection guide 720 and engage with one or more landmarks of a bone or joint or anatomical structure of a patient. Registration of the landmark registration feature 728 to a landmark of a bone or joint can serve to confirm and/or ensure that a surgeon has located a desired placement and/or orientation for a resection guide 720.

In certain embodiments, the bone engagement surfaces 726 are patient-specific: contoured to match a surface of: one or more bones and/or bone surfaces the resection guide 720 contacts during the procedure or one or more joints proximal to the resection guide 720 during the procedure. Alternatively, or in addition, the bone engagement surface 726 may not be patient-specific, and may, or may not, contact a bone surface during use of the resection guide 720. In one embodiment, a skin contact surface may be used in addition to, or in place of, a bone engagement surface. Those of skill in the art appreciate that one or more sides of any of the members of the system 700 may include one or more bone engagement surfaces 726. Consequently, one or more sides of the fasteners 710, the resection guide(s) 720, the complementary components 730, navigation guides 792, and/or the implants 794 may include one or more bone engagement surfaces 726.

In certain embodiments, the resection guides 720 and/or aspects of the resection guides 720 may be integrated into other components and/or instruments, such as a pin guide, a trajectory guide, an alignment guide, or the like.

The complementary components 730 serve to assist a surgeon during one or more steps of a procedure. Those of skill in the art appreciate that a number of components can serve as complementary components 730. One or more of the features, functions, or aspects of the complementary components 730 can include patient-specific features.

Examples of complementary components 730 include, but are not limited to, an alignment guide 740, a rotation guide 750, a reduction guide 760, a compression guide 770, a positioning guide 780, a fixation guide 790, navigation guides 792, and/or one or more implants 794. In general, the complementary components 730 serve to assist a surgeon in performing the function included in the name of the complementary component 730. Thus, an alignment guide 740 can help a surgeon align bones, parts of bones, or other parts of a patient as part of a procedure. A rotation guide 750 can help a surgeon rotate one or more bones, parts of bones, or other parts of a patient as part of a procedure. In one embodiment, a rotation guide 750 may hold one bone fragment stable while another bone fragment is rotated into a desired position.

A reduction guide 760 can help a surgeon position and/or orient one or more bones, parts of bones, or other parts of a patient as part of a procedure in order to reduce the bone, bones, bone parts, or other parts and/or in order to position and/or orient the bone, bones, bone parts, or other parts to a desired position and/or orientation. In certain embodiments, aspects and/or features of a reduction guide 760 can be integrated into one or more other components of an osteotomy system 700, such as components of the complementary components 730. A compression guide 770 can help a surgeon compress one or more bones, parts of bones, or other parts of a patient together or against an implant as part of a procedure. In certain embodiments, compression guide 770 can be a separate instrument such as a compressor and/or a combined compressor/distractor. The compressor/distractor can be used to compress two or more cut faces formed by an osteotomy until fixation is deployed or distract bones or parts of bones involved in a procedure. In certain embodiments, a compression guide 770 may serve a dual purpose as both a compression guide 770 and as a positioning guide 780. The same instrument may be used to both translate and/or rotate bones or bone fragments and compress two or more cut faces formed by an osteotomy until fixation can be deployed.

A positioning guide 780 (also referred to as a positioner) can help a surgeon position one or more bones, parts of bones, or other parts of a patient as part of a procedure. For example, a positioning guide 780 may hold one bone or bone fragment stable and hold one or more other bone fragments in a desired position while permanent or temporary fixation is deployed. In certain embodiments, the positioning guide 780 may hold bone fragments in a reduced position, and thus may function as both a positioning guide 780 and/or a reduction guide 760.

In certain embodiments, the positioning guide 780 may be designed and fabricated to be patient-specific. The patient-specific aspects can include a patient-specific bone engagement surface, a predefined angle for reorienting one or more bone or bone parts within one or more planes, a predefined position for bone attachment features 724 or fasteners 710, a predefined or patient-specific offset or amount of translation that is provided, or the like. Alternatively, or in addition, the positioning guide 780 may be selected from a kit, collection, or repository of a number of positioning guides 780: each having a different configuration for one or more aspects/attributes of the positioning guide 780. For example, each member of the repository/kit may include a different positioning angle (repositioning or correction angle), the angles may differ by 2 degrees for example. In such an embodiment, each positioning guide 780 may not be patient-specific to a particular patient but may provide the desired amount of positioning to meet the goals of the surgeon. In certain embodiments, a preoperative plan generated based on the present disclosure may include a recommendation for the positioning guide 780 to be used, even if the recommended positioning guide 780 is not patient-specific to the particular patient.

A fixation guide 790 can help a surgeon in completing one or more temporary or permanent fixation steps for one or more bones, parts of bones, or other parts of a patient as part of a procedure. The fixation guide 790 may include and/or may use one or more components of a fastener or fixation system including implant hardware of the fastener or fixation system.

Those of skill in the art will appreciate that the other complementary components 730 may each have functions, purposes, and/or advantages with respect to one or more anatomical parts of the patient. Alternatively, or in addition, the other complementary components 730 may each have functions, purposes, and/or advantages with respect to one or more instruments and/or one or more anatomical parts of the patient. For example, a trajectory guide may be a type of alignment guide 740 in that the trajectory guide facilitates alignment of fixation with the desired location and/or trajectory/orientation with respect to one or more anatomical parts of the patient. Alternatively, or in addition, a trajectory guide may also be considered a type of fixation guide 790 because the trajectory guide facilitates deployment of one or more fasteners 710.

Advantageously, the system 700 can help a surgeon overcome one or more of the challenges in performing an osteotomy procedure, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the fasteners 710, resection guide(s) 720, and/or complementary components 730 can be configured to assist in overcoming this challenge.

Advantageously, system 700 can help a surgeon in positioning, placing, and/or orienting a resection guide accurately. Modern techniques may include preoperative planning, simulation, or even practice using computer models, 3D printed models, virtual reality systems, augmented reality systems or the like. However, simulations and models are still different from actually positioning a resection guide on a patient's bone, joint, or body part during the procedure. System 700 can include a number of features, including patient-specific features, to assist the surgeon with the positioning. In one embodiment, the resection guide 720 can include one or more landmark registration features 728.

Advantageously, the system 700 can help a surgeon in securing guides of the osteotomy system 700, such as a resection guide, as well as how to readily remove the guide (e.g., resection guide) without disturbing a reduction, shifting, reorienting, or repositioning one or more bones or parts of bones while removing the guide. In certain embodiments, the system 700 is configured to permit removal of a guide while keeping temporary fasteners in place for use in subsequent steps of an osteotomy procedure. Alternatively, or in addition, system 700 may facilitate positioning of temporary fasteners during one step of a wedge osteotomy procedure for use in a subsequent step of the wedge osteotomy procedure. Removal of a guide during an osteotomy procedure can be particularly challenging where translation and/or rotation of the bones involved in the osteotomy procedure is required for the success of the osteotomy procedure. Advantageously, system 700 accommodates translation and/or rotation of the bones during the osteotomy procedure while facilitating a successful outcome for the osteotomy procedure.

Advantageously, the components of the system 700 can be specifically designed for a particular patient. Alternatively, or in addition, the components of the system 700 can be specifically designed for a class of patients. Each of the components of system 700 can be designed, adapted, engineered and/or manufactured such that each feature, attribute, or aspect of the component is specifically designed to address one or more specific indications present in a patient. Advantageously, the cuts made for the osteotomy procedure can be of a size, position, orientation, and/or angle that provides for an optimal osteotomy with minimal risk of undesirable resection. In one embodiment, the components of system 700 can be configured such that an osteotomy is performed that enables a correction in more than one plane in relation to the parts of the body of the patient. For example, cut channels or resection features 722 in a resection guide 720 can be oriented and configured such that when the bones are fused/fixated the correction results from translation, rotation, and/or movement of bones or bone parts in two or more planes (e.g., sagittal and transverse) once the fragments or bones are reduced.

In certain embodiments, the exemplary system 700 may include a plurality of fasteners 710, resection guides 720, and/or complementary components 730. For example, a surgeon may plan to resect a plurality of osteotomies from the bone(s) in order to accomplish a desired correction. In one example, one or more wedge segments may be resected from a medial side of a patient's foot and another one or more wedge segments may be resected from a lateral side of the patient's foot. These wedge segments may extend part way into the foot, or through from one side of the foot to the other. Of course, multiple wedge segments may be formed on one side of the foot as well.

Additionally, a surgeon may use one or more components in an exemplary system 700 to make multiple cuts in the bone(s). The multiple cuts may be centered over or around an apex of a deformity or positioned at other locations within the foot such that when the multiple cuts are made, any resected segments removed, or added bone void fillers introduced, and/or bones and/or bone fragments translated and/or rotated the combined angles, surfaces, removed segments, and/or added portions cooperate to provide a desired correction. Each of the components of the exemplary system 700 can be identified, defined, and reviewed using the apparatuses, systems, and/or methods of the present disclosure.

In certain embodiments, the components of system 700 may be made as small as possible to minimize the amount of soft tissue that is opened in the patient for the osteotomy procedure. Alternatively, or in addition, walls and/or sides of the components may be beveled and/or angled to avoid contact with other hard tissue or soft tissues in the operating field for the osteotomy procedure.

Those of skill in the art will appreciate that for certain osteotomy procedures a complementary component 730 may not be needed or a given complementary component 730 may be optional for use in the osteotomy procedure. Similarly, those of skill in the art will appreciate that certain features of the fasteners 710, resection guides 720, and/or complementary components 730 can be combined into one or more of apparatus or devices or may be provided using a plurality of separate devices.

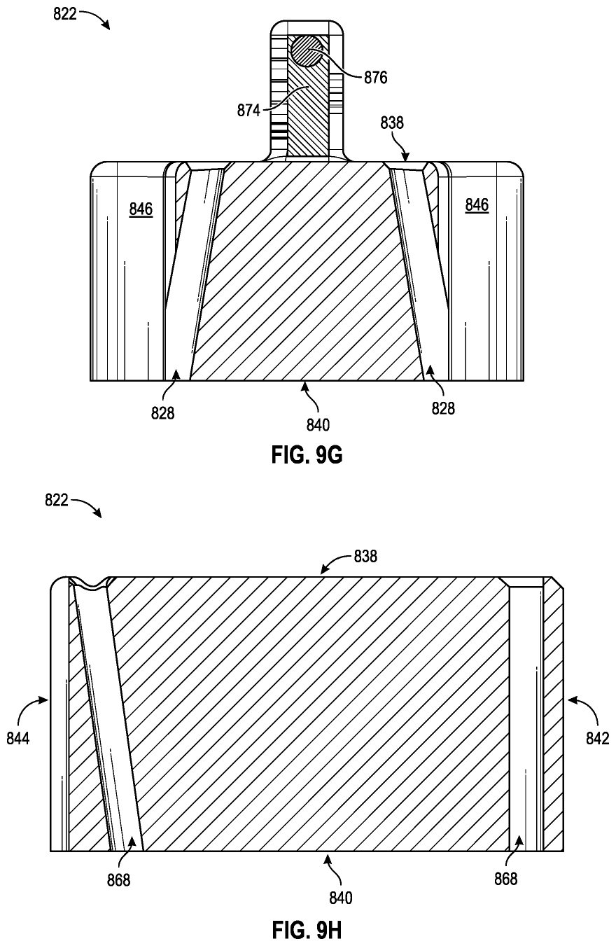

FIG. 8 illustrates an exemplary ankle fusion osteotomy system 800, according to one embodiment. The osteotomy system 800 can include one or more fasteners 810 and/or two or more sets of fasteners (810a, 810b, 810c, 810d), one or more resection guides 820, and zero or one or more complementary components 730. In the illustrated embodiment, the resection guides 820 include a tibial resection guide 822 and a talus resection guide 824. The resection guides 820 may also include one or more of resection features 826, bone attachment features 828, bone engagement feature 830, and/or landmark registration feature 834. In certain embodiments, a bone engagement feature 830 can include a bone engagement surface 832.

While specific embodiments of complementary components 730 are not specifically shown here in relation to the osteotomy system 800, those of skill in the art will appreciate that complementary components 730 can be similar in feature, design, implementation, configuration, and purpose as those described in relation to the osteotomy system 700 and can be used for the osteotomy system 800. Thus, the osteotomy system 800 can include one or more alignment guides 740, rotation guides 750, correction guides 760, compression guides 770, positioning guides 780, fixation guides 790, navigation guides 792, implants 794, or the like.

Either or both of the resection guides 820 may be custom patient-specific resection guides made for a particular patient and/or for a particular surgical procedure. Various aspects of the resection guides 820 may be patient-specific, including, but not limited to, an angle and/or orientation for a resection feature of the resection guide 820, a position of the resection feature, a depth of the resection feature, a size of the resection guide 820, a configuration and/or composition of a bone contacting surface such as a bone engagement surface of the resection guide 820, and the like.

In one embodiment, the tibial resection guide 822 includes a body 836 that includes an anterior side 838, a posterior side 840, a medial side 842, a lateral side 844, a superior side 846, and an inferior side 848. The talus resection guide 824 may also include a body 850 that includes an anterior side 852, a posterior side 854, a medial side 856, a lateral side 858, a superior side 860, and an inferior side 862.

In the illustrated embodiment, the tibial resection guide 822 and talus resection guide 824 are separate instruments. Advantageously, using two separate resection guides can enable a surgeon to adapt intraoperatively to an amount of rotation and/or translation of one or more bones the soft tissue around the bones will permit. Specifically, one resection guide (e.g., tibial and/or talus) may include a resection feature with a first angle for correction, a second resection guide may include a resection feature with a second angle for correction greater than the first, a third resection guide may include a resection feature with a third angle for correction greater than the second, and so forth. These different resection guides may be part of a kit available to the surgeon during the surgery. In this manner, a surgeon can choose a resection guide with a preferred angle for correction during the surgery.

In another embodiment, the tibial resection guide 822 and talus resection guide 824 and/or components of the tibial resection guide 822 and talus resection guide 824 may be combined into a single instrument. In another embodiment, the tibial resection guide 822 and/or talus resection guide 824 may include a coupler that enables the tibial resection guide 822 and talus resection guide 824 to be coupled together before or during a surgical procedure.

In certain embodiments, a position of one or more of a resection feature 826 of the tibial resection guide 822 and/or a resection feature 826 of the talus resection guide 824 can be determined and/or defined at least partially based on a bone model of at least a portion of a bone of a patient's foot. In one embodiment, the resection features 826 may be positioned to remove a minimal amount of bone and implement a deformity correction.

In one embodiment, the position and/or configuration of the resection feature 826 may be determined, defined, and/or dictated by both a bone model of a portion of a patient's foot and user instructions 604. Advantageously, the user instructions 604 may identify certain goals for the resection feature 826 such as minimal size (i.e., width, length, depth), shape, contour, position, orientation, trajectory, and the like. Since the resection feature 826 guides the formation of an osteotomy, the configuration of the resection feature 826 provided by the user instructions 604 can also define an osteotomy created using the resection feature 826.

The resection feature 826 of the tibial resection guide 822 may be referred to as a tibia resection feature 864 and the resection feature 826 of the talus resection guide 824 may be referred to as a talus resection feature 866. In certain embodiments, this means that a user, or end user, may review the bone model, and a model of the tibial resection guide 822 and/or the talus resection guide 824 and based on these models determine, at least in part, where to position the resection feature 826 (e.g., tibia resection feature 864 and talus resection feature 866) within a resection guide 820. Advantageously, by reviewing and working with the models, a user and/or end user can minimize the amount of bone removed to perform a successful surgical procedure. Determining a position for the resection features 826 in a model of an instrument can be directly reflected in a fabricated instrument based on the model. In one embodiment, the position of a resection feature can be based on user instructions 604. Alternatively, or in addition, other features of a resection guide 820 can be based, at least partially, on user instructions 604.

In one embodiment, the tibia resection feature 864 is configured to guide a cutting tool to form a first osteotomy in a tibia of a patient. The talus resection feature 866 is configured to guide the cutting tool to form a second osteotomy in a talus of the patient. The first osteotomy and second osteotomy are configured to cooperate with each other to form a resection interface between the tibia and the talus of the patient. The resection interface is an interface between a resected portion of the tibia and a resected portion of the talus. In certain embodiments, these resected portions (e.g., cut faces) are brought into contact and fixation is deployed to promote fusion of the talus to the tibia at the resection interface. In certain embodiments, the resection interface includes no implant or other structure between the resected portion of the tibia and the resected portion of the talus. In contrast to an arthroplasty surgical procedure, the resection guides 820 are used for a bone fusion procedure, an arthrodesis. Thus, the interface in the embodiments of the present disclosure are for facilitating a joint fusion.

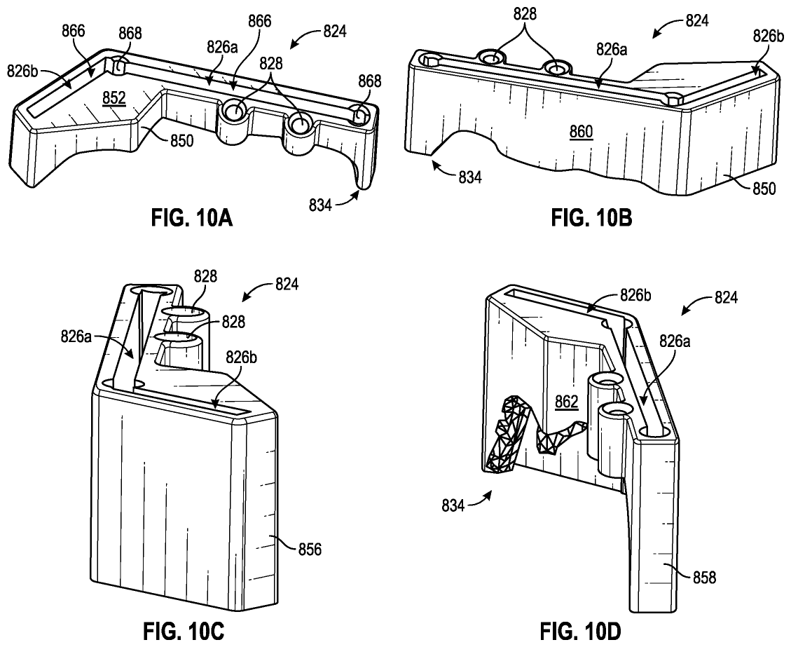

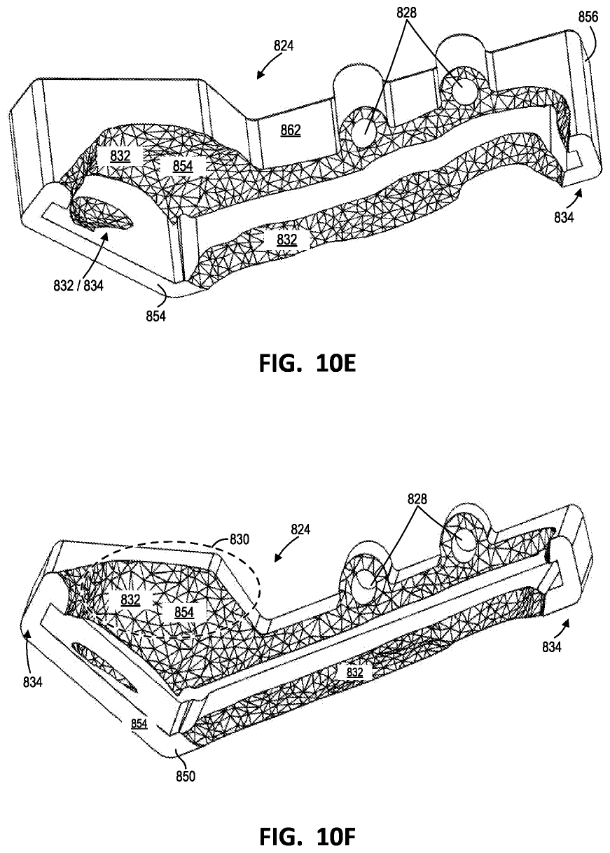

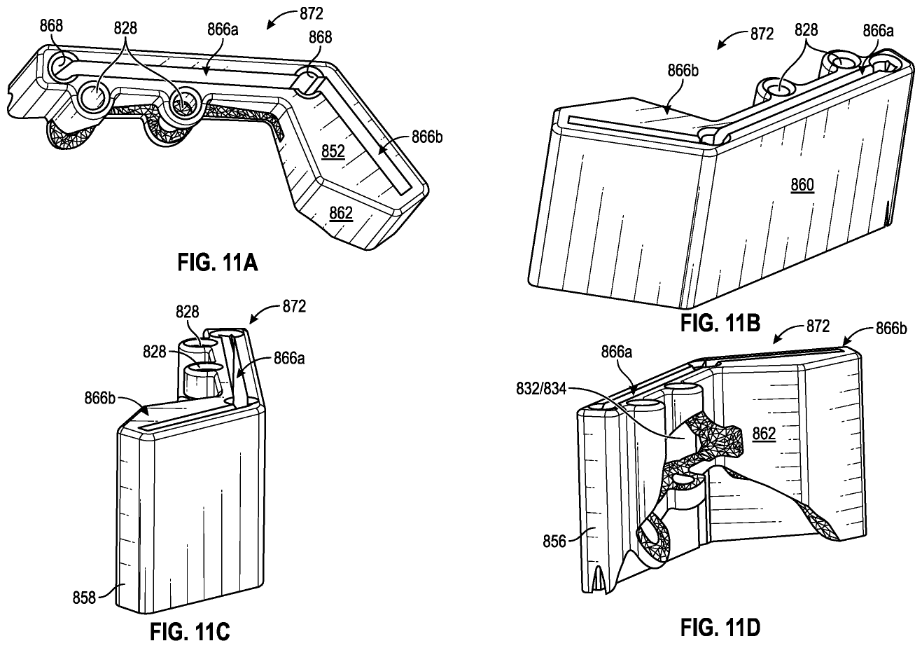

FIGS. 9A-9F illustrate views of a resection guide 820 (e.g., tibial resection guide 822) of the osteotomy system of FIG. 8, according to one embodiment. The tibial resection guide 822 includes one or more resection features 826, one or more bone attachment features 828 (e.g., a fastener 810 and opening), and one or more bone engagement features 830 which may include one or more bone engagement surfaces 832. In certain embodiments, bone engagement features 830 implemented in a tibial resection guide 822 are referred to as tibial bone engagement features.

Advantageously, the resection features 826 can be positioned, sized, and/or oriented to enable a surgeon to resect any particular shape in the bone for the osteotomy procedure. In the illustrated embodiment, the resection features 826 are configured to direct a cutting tool at an angle in one or more planes into the bone. The angle may be normal to a longitudinal axis of the bone, at an oblique angle, at an acute angle, or at an obtuse angle relative to a longitudinal axis of the bone. Advantageously, the size, shape, and angle of the resection can be predefined and can be determined preoperatively and/or can be patient-specific. In manner, the tibial resection guide 822 serves to provide for a patient-specific osteotomy procedure. Alternatively, or in addition, the resection features 826 can be configured to enable a surgeon to readily resect in a plantar direction and/or a dorsal direction. In certain embodiments, the resection features 826 may include an opening on one end or the other or both ends to permit the surgeon to position a cutting tool to make desired cuts that can extend laterally or medially.

In addition, the tibial resection guide 822 includes an anterior side 838, posterior side 840, a medial side 842, lateral side 844, superior side 846, and an inferior side 848. Generally, the sides of the tibial resection guide 822 refer to the direction the sides face when the resection guide 820 is in use. In certain embodiments, the tibial resection guide 822 can include a landmark registration feature 834.

In the illustrated embodiment, at least one landmark registration feature 834 extends from the posterior side 840. Advantageously, the landmark registration feature 834 can provide a surgeon with confidence and assurance in the placement and positioning of the tibial resection guide 822 on the bone because the landmark registration feature 834 can be configured to engage with a particular landmark on the bone (e.g., a projection or a depression or cavity). Alternatively, or in addition, the landmark registration feature 834 can include a contoured bone engagement surface 832 that can further facilitate registration of the landmark registration feature 834 and/or tibial resection guide 822 with the bone. In this manner, a surgeon can be assured intraoperatively that the tibial resection guide 822 is being positioned as desired.

In certain embodiments, the landmark registration feature 834 can be shaped like a hook to engage a surface or structure of a bone. Alternatively, or in addition, the tibial resection guide 822 may include a landmark registration feature 834 on each end (lateral and medial), together the landmark registration features 834 can engage one or more landmarks of a surface of the bone such that the surgeon can accurately position and register the tibial resection guide 822 to the bone.

In the illustrated embodiment, the tibial resection guide 822 is shaped such that the superior side 846 is not as wide as the inferior side 848. This shape can be advantageous because this enables the tibial resection guide 822 to be smaller towards the proximal end of the tibia 226. The inferior side 848 may be just large enough to support the resection feature 826. This triangular shape of the tibial resection guide 822 allows for a minimal length and width for the opening in the skin and soft tissue of the patient in order to secure the tibial resection guide 822 to the tibia 226.

FIGS. 9E and 9F illustrate the bone engagement surface 832 of an example tibial resection guide 822. The bone engagement surface 832 facilitates registration of the tibial resection guide 822 to the tibia 226. The position of the tibial resection guide 822 on a distal end of the tibia 226 may be determined by a surgeon preoperatively.

FIG. 9F illustrates that the bone engagement surface 832 may be part of a tibial bone engagement feature 830 which may be part of a tibial resection guide 822. A bone engagement feature is any structure configured to engage or assist in the engagement of one or more bones. Thus, any structure that engages or assists in the engagement of a resection guides 820 with a bone is a bone engagement feature.

FIG. 9F illustrates two examples of a bone engagement feature 830, tibial bone engagement feature of the tibial resection guide 822. In the illustrated embodiment, the bone engagement feature 830 includes a body section (i.e. part of the body 836) that is coupled to and/or supports a bone engagement surface 832. The bone engagement surface 832 is configured to register to (i.e. seated or fit with) a surface of the tibia, directly below and/or in contact with the body section when the tibial resection guide 822 is in use. As one example, this means that projections from the surface of the tibia 226 fit within voids in the bone engagement surface 832 when the tibial resection guide 822 is in use and in a desired position relative to a position of a model of the tibial resection guide 822 in relation to a model of a patient's tibia 226.

FIG. 9F illustrates that structurally, the tibial resection guide 822 may be able to function without the body sections identified as part of the bone engagement features 830. However, in the illustrated embodiment, the size (length, width, height) of the tibial resection guide 822 may be determined, defined, and/or dictated at least in part based on user instructions 604. Thus, the surgeon may also provide instructions for the number, size, and/or configuration of bone engagement feature 830 of the tibial resection guide 822. The bone engagement features 830 may not be needed structurally, however, they may be very helpful to a surgeon in positioning the tibial resection guide 822 in a desired position relative to a bone of a patient.

These user instructions 604 may have initiated the design and/or fabrication of the tibial resection guide 822. For example, a surgeon may have desired to have as much surface area on the posterior side 840 as possible while still keeping the overall size of the tibial resection guide 822 as small as practicable. Accordingly, in the illustrated embodiment, a surgeon may have indicated that two bone engagement feature 830 that includes bone engagement surface 832 are to be positioned and sized as indicated between the bone attachment features 828 and the tibia resection feature 864. In this manner, a surgeon may maximize an amount of surface area of the tibial resection guide 822 that contacts a tibia 226 to facilitate initial positioning intraoperatively to match or substantially match a position planned using a model of the tibial resection guide 822 and a model of the tibia 226. In certain embodiments, a surgeon may, at their discretion, include in the user instructions 604, instructions to add another body section that includes a bone engagement surface 832 to a medial side 842, a lateral side 844, a superior side 846, and/or an inferior side 848 of the tibial resection guide 822.

FIG. 9G illustrates a cross section view of the tibial resection guide 822 in FIG. 9F taken along line 9G. FIG. 9G illustrates angles for bone attachment features 828 (e.g., fasteners 810 in openings). In the illustrated embodiment, the angles cause the bone attachment features 828 to diverge as they extend into the bone. Advantageously, a surgeon may predefine an angle for one or more bone attachment features 828 preoperatively.

FIG. 9H illustrates a cross section view of the tibial resection guide 822 in FIG. 9F taken along line 9H. FIG. 9H illustrates angles for openings 868 that can cooperate with fastener 810 to provide resection guards 870. In the illustrated embodiment, the angle of the opening 868 nearest the lateral side 844 starts on the anterior side 838 and extends medially through the tibial resection guide 822 such that the fastener 810 extends at this same angle into the tibia 226. In contrast, the angle of the opening 868 nearest the a medial side 842 starts on the anterior side 838 and extends perpendicular to the anterior side 838 through the tibial resection guide 822 such that the fastener 810 extends at this same angle into the tibia 226. Advantageously, a surgeon may predefine one or more of these angles for resection guards 870 preoperatively.

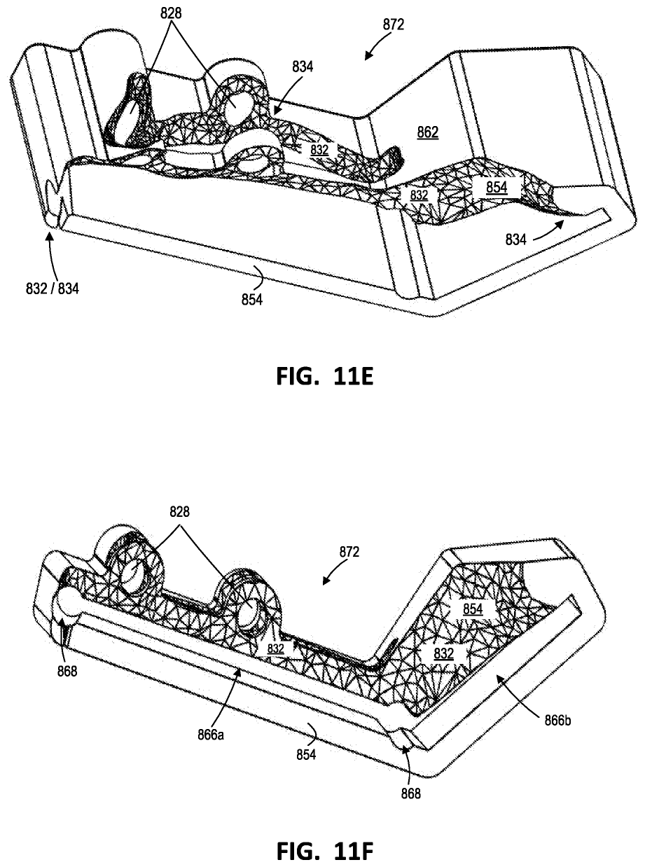

FIGS. 10A-10F illustrate views of a resection guide 820 (e.g., talus resection guide 824) of the osteotomy system of FIG. 8, according to one embodiment. The talus resection guide 824 includes one or more resection features 826 (e.g., resection features 826a-b), one or more bone attachment features 828 (e.g., a fastener 810 and opening), and one or more bone engagement features 830, which may include a bone engagement surface 832. In certain embodiments, bone engagement features 830 implemented in a talus resection guide 824 are referred to as talus bone engagement features.

FIG. 10F illustrates that the bone engagement surface 832 may be part of a bone engagement feature 830 which may be part of a talus resection guide 824. In the illustrated embodiment, the bone engagement surface 832 may be part of a talus bone engagement feature 830. FIG. 10F illustrates one example of a bone engagement feature 830, talus bone engagement feature of the talus resection guide 824. In the illustrated embodiment, the bone engagement feature 830 includes a body section (i.e. part of the body 850) that is coupled to and/or supports a bone engagement surface 832. The bone engagement surface 832 is configured to register to (i.e. seated or fit with) a surface of the talus, directly below and/or in contact with the body section when the talus resection guide 824 is in use. As one example, this means that projections from the surface of the talus 222 fit within voids in the bone engagement surface 832 when the talus resection guide 824 is in use and in a desired position relative to a position of a model of the talus resection guide 824 in relation to a model of a patient's talus 222.

FIG. 10F illustrates that structurally, the talus resection guide 824 may be able to function without the body section identified as part of the bone engagement features 830. However, in the illustrated embodiment, the size (length, width, height) of the talus resection guide 824 may be determined, defined, and/or dictated at least in part based on user instructions 604. Thus, the surgeon may also provide instructions for the number, size, and/or configuration of bone engagement feature 830 of the talus resection guide 824. The bone engagement features 830 may not be needed structurally, however, they may be very helpful to a surgeon in positioning the talus resection guide 824 in a desired position relative to a bone of a patient.

These user instructions 604 may have initiated the design and/or fabrication of the talus resection guide 824. For example, a surgeon may have desired to have increased surface area on the posterior side 854 and medial side of the talus resection guide 824 as possible while still keeping the overall size of the talus resection guide 824 as small as practicable. Accordingly, in the illustrated embodiment, a surgeon may have indicated that one bone engagement feature 830 that includes bone engagement surface 832 is to be positioned and sized as indicated on the medial side between the resection feature 826a and the resection feature 826b. In this manner, a surgeon may selectively include surface area on the posterior side 854 of the talus resection guide 824 that contacts a talus 222 to facilitate initial positioning intraoperatively to match or substantially match a position planned using a model of the talus resection guide 824 and a model of the talus 222. In certain embodiments, a surgeon may, at their discretion, include in the user instructions 604, instructions to add another body section that includes a bone engagement surface 832 to a lateral side 858, a medial side 856, superior side 860, and/or inferior side 862 of the talus resection guide 824.

The resection features 826a,b can be similar or the same as the resection feature 826 described herein. In addition, the talus resection guide 824 includes an anterior side 852, a posterior side 854, a medial side 856, a lateral side 858, a superior side 860, and an inferior side 862. Generally, the sides of the talus resection guide 824 refer to the direction the sides face when the talus resection guide 824 is in use. In certain embodiments, the talus resection guide 824 can include a landmark registration feature 834.

In the illustrated embodiment, at least one landmark registration feature 834 extends from the posterior side 854. Advantageously, the landmark registration feature 834 can provide a surgeon with confidence and assurance in the placement and positioning of the talus resection guide 824 on the bone because the landmark registration feature 834 can be configured to engage with a particular landmark on the bone (e.g., a projection or a depression or cavity). Alternatively, or in addition, the landmark registration feature 834 can include a contoured bone engagement surface that can further facilitate registration of the landmark registration feature 834 and/or talus resection guide 824 with the bone. In this manner, a surgeon can be assured intraoperatively that the tibial resection guide 822 is being positioned as desired.

In certain embodiments, the landmark registration feature 834 can be shaped like a hook to engage a surface or structure of a bone. Alternatively, or in addition, the talus resection guide 824 may include a landmark registration feature 834 on each end (lateral and medial), together the landmark registration features 834 can engage one or more landmarks of a surface of the bone such that the surgeon can accurately position and register the talus resection guide 824 to the bone.

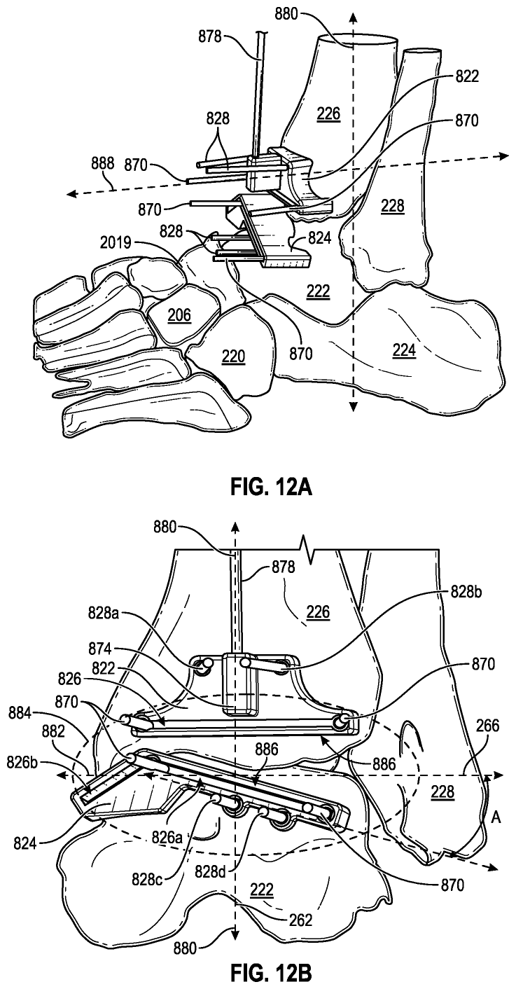

FIGS. 11A-11F illustrate views of a talus resection guide 872 of an osteotomy system, according to an alternative embodiment. It should be noted that the talus resection guide 872 of FIGS. 11A-F is a talus resection guide 872 that is similar or the same as the talus resection guide 824 described above, with the main difference being the talus resection guide 872 may be for a right ankle of a patient. Alternatively, or in addition, the talus resection guide 872 may be for a left ankle of a patient but the resection features 826 may be different from talus resection guide 824 due to user instructions 604 for a particular surgical procedure.

FIGS. 10E, 10F, 11D, 11E, and 11F illustrate a bone engagement surface 832 of an example talus resection guide 824. The bone engagement surface 832 facilitates registration of the talus resection guide 824 to the talus 222. The position of the talus resection guide 824 on a proximal end of the talus 222 may be determined by a surgeon preoperatively. FIGS. 10E and 10J illustrate examples of a structure of a talus resection guide 824 that can serve as a landmark registration feature 834 and/or a bone engagement surface 832. The landmark registration feature 834 may form an indentation or pocket within a side wall of the talus resection guide 824. Whether or not a talus resection guide 824 includes a pocket shaped landmark registration feature 834 can depend on the anatomy of the patient and the desired position and orientation of the talus resection guide 824 as overseen by the surgeon.

Those of skill in the art will appreciate that the talus resection guide 824/872 and/or tibial resection guide 822 can be designed, engineered, organized, and/or fabricated using one or more of methods and/or processes described herein.

As explained, the exemplary ankle fusion osteotomy system 800 can include one or more complementary components 730 which serve to assist a surgeon during one or more steps of an osteotomy procedure. Those of skill in the art appreciate that a number of components can serve as complementary components 730. Examples of complementary components 730 include, but are not limited to, an alignment guide 740, a rotation guide 750, a correction guide 760, a compression guide 770, a positioning guide 780, a fixation guide 790, and one or more implants 796.

Furthermore, those of skill in the art appreciate that certain complementary component 730 may include one or more pins, fasteners 810, or other complementary component 730 that are designed and positioned to facilitate completing a surgical procedure. For example, certain pins or fasteners 810 may remain engaged with bones (prior to resection or after resection) to assist a surgeon in translating, compressing, rotating, and/or attaching fixation hardware to complete a surgical procedure.

In one embodiment, the exemplary ankle fusion osteotomy system 800 may include one or more drill guides for pilot or anchor holes for one or more fasteners of a fixation assembly. For example, holes extending from the anterior side 852 to the posterior side 854 may be used for drill pilot holes and/or K-wires for deployment of bone screws and/or a bone plate.

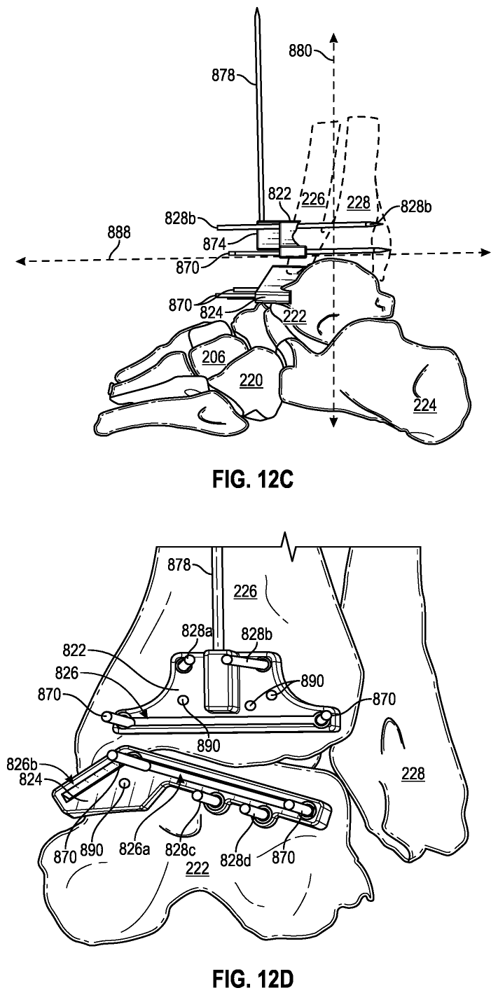

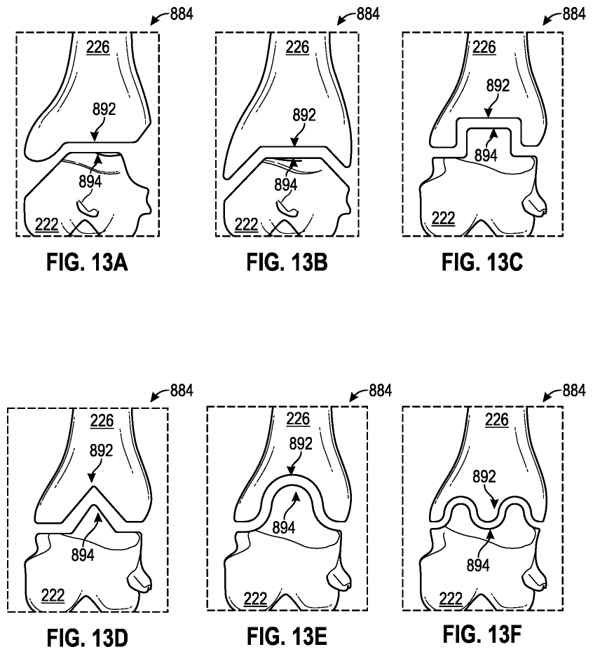

FIGS. 12A-12C illustrate different views a surgical osteotomy procedure using the osteotomy system of FIG. 8, according to one embodiment.

FIG. 12A illustrates a stage of performing a surgical osteotomy procedure (e.g., tibia and/or talus osteotomy and/or ankle fusion) using the osteotomy system 800, according to one embodiment. The exemplary ankle fusion osteotomy system 800 can be used to perform osteotomies for a surgical procedure that includes one or more of a fusion of a joint (e.g., arthrodesis), joint replacement, such as a total ankle replacement, and/or correction of a deformity. A surgeon may elect to approach the osteotomies on the anterior side of the foot. FIG. 12A illustrates a left foot and shows a lateral perspective view and an anterior-posterior axis 888. FIG. 12A illustrates two resection guides 820 (e.g., tibial resection guide 822 and talus resection guide 824) one secured to the talus 222 and one secured to the tibia 226 by way of one or more bone attachment features 828 which may be implemented, at least in part, using fasteners 810 and/or one or more holes or openings in the tibial resection guide 822 and talus resection guide 824.

FIG. 12B illustrates a close up anterior view of the talus resection guide 824 and tibial resection guide 822 positioned on the talus 222 and the tibia 226 of the left foot. Other foot bones are not shown for clarity. The talus resection guide 824 and/or tibial resection guide 822 may each include one or more resection features 826. The resection features 826 may serve similar, or the same, purposes as the resection features 722 and/or other resection features described herein. The resection features 826 guide a cutting tool in performing an osteotomy. Advantageously, as with other embodiments described herein, the number, size, configuration, length, width, position, and/or angle in one or more planes of the resection features 826 can be defined for a particular patient or for a group of patients and can be included in the tibial resection guide 822 and/or resection guard 870 provided for a surgical procedure.

The bone attachment features 828 secure the resection guide 820 to the bone for performing an osteotomy. Advantageously, as with other embodiments described herein, the number, size, configuration, length, width, position, and/or angle in one or more planes of the bone attachment features 828 can be defined for a particular patient or group of patients. In certain embodiments, one or more of the bone attachment features 828 (e.g., bone attachment features 828a, b, c, d) may be configured to enter the bone at predefined angles such that the pins, K-wires, or fasteners used as part of the bone attachment features 828 diverge, converge, or are parallel to each other as they extend into the bone.

FIG. 12B illustrates that the bone attachment features 828 of the tibial resection guide 822 may be angled such that the fasteners diverge as they extend into the tibia 226. This divergence can be advantageous since it can provide a stable engagement between the resection guide 820 and the bone. Similarly, converging bone attachment features 828 as they extend into the bone can also provide stable engagement of the bone. Alternatively, or in addition, divergence and/or convergence of one or more bone attachment features 828 can minimize the number of one or more bone attachment features 828 used for a surgical procedure. This can save time and materials. In the illustrated embodiment, bone attachment feature 828a may be angled medially and bone attachment feature 828b may be angled laterally. In another embodiment, bone attachment feature 828a may be angled to enter the bone perpendicular to surface and bone attachment feature 828b may be angled to converge with bone attachment feature 828a (either outside or within the bone). Advantageously, a surgeon can request certain configurations and/or trajectories for the bone attachment features 828 (e.g., by way of the user instructions 604).

Those of skill in the art will appreciate that the talus resection guide 824 and/or the tibial resection guide 822 may include more or fewer bone attachment features 828 than those included in the illustrated embodiments. For example, in one embodiment, the talus resection guide 824 may include one or more additional bone attachment features 828 near a medial side 856 and/or near resection feature 826b. In certain embodiments, the number of bone attachment features 828 may be determined at least in part by user instructions 604.

In the illustrated embodiment, the talus resection guide 824 includes two resection features 826 (resection feature 826a, resection feature 826b). Those of skill in the art will appreciate that a single resection feature 826 can be used instead of two or more resection features 826. In one embodiment, the resection feature 826b can assist a surgeon in resecting hard and/or soft tissue on a medial side of the joint. Such resection can contribute to a successful surgical procedure. In one embodiment, such resection may create clearance on the talus 222 for the talus 222 to fit under a medial malleolus when fused to the tibia 226. In certain embodiments, a resection feature 826 can be included on the lateral side of the talus resection guide 824. Similarly, additional resection features 826 can be used on the tibial resection guide 822 as well.

The resection feature 826a may be used to make a primary resection of the talus 222. The resection feature 826b may be used to make a secondary, or additional resection, along a side of the talus 222 (e.g., a medial side of the talus 222). During the surgical procedure, a surgeon may resect within the resection feature 826b to remove medial parts of the talus 222 and/or parts of the medial malleolus (e.g., within a medial gutter between the tibia 226 and the talus 222) to facilitate a clean and accurate fit between the resected talus 222 and the resected tibia 226 during reduction and for the fusion.

In certain embodiments, the resection feature 826a can be angled in a cephalad/dorsal direction or a caudal/plantar direction within the transverse plane 266. Alternatively, or in addition, the resection feature 826a can be angled in a cephalad/dorsal direction or a caudal/plantar direction within the sagittal plane 262. Alternatively, or in addition, the resection feature 826a can be positioned to resect the talus 222 normal to the transverse plane 266.

Advantageously, a surgeon (e.g., end user) and/or fabricator of the resection guides 820 can predefine desired angles, locations, and sizes for any, each, or all of the resection features 826 of the resection guides 820. For example, size, angle, orientation, trajectory, and location for any of the resection feature 826 can be determined by way of the user instructions 604. In this manner, a surgeon can resect the tibia 226 and/or talus 222 in such a way that positions the talus 222 fore, aft, medial, or lateral within the joint when fused to correct a deformity.

In addition, the ability to define resection features 826 preoperatively can enable a surgeon to resect one or more malleoli of a patient to achieve a desired surgical procedure outcome. For example, the resection guides 820 can be used to resect the malleoli of a patient such that translational/rotational correction in one joint or foot match another joint or foot or extremity more precisely.

In the illustrated embodiment, the resection feature 826a extends in a plantar direction away from a medial-lateral axis 882 at an angle A measured in degrees. The angle A may range from 1 degree to 60 degrees. A surgeon may designate and/or revise the magnitude of angle A, until the result of the osteotomies using angle A will provide a desired level of correction as determined by the surgeon. In certain embodiments, the osteotomy system 800 may include a proposed magnitude for angle A, that a surgeon can then revise as needed or desired.

In the illustrated embodiment, the resection feature 826a may extend at angle A in relation to the medial-lateral axis 882 and guide a cutting tool perpendicular to a mechanical axis 880 of the tibia 226. The medial-lateral axis 882 runs in the transverse plane 266 perpendicular to the mechanical axis 880 of the tibia 226.

Once a first osteotomy is performed using resection feature 826 of the tibial resection guide 822 and a second osteotomy is performed using the resection feature 826 (e.g., 826a, 826b) of the talus resection guide 824, a surgeon can then reduce the osteotomies. When a surgeon reduces the osteotomies and abuts the first osteotomy against the second osteotomy, the reduced osteotomies remediate a deformity of the patient. The tibia 226 and talus 222 can now fuse to complete the arthrodesis. It should be noted that in the illustrated embodiment the position, alignment, orientation, and/or trajectories for the resection features 826 are selected for an arthrodesis, no implants will be used between the first osteotomy and the second osteotomy.

FIG. 12B illustrates a resection interface 884 (see dashed oval). Prior to the osteotomies, the resection interface 884 can include a distal end of the tibia 226 and a proximal end of the talus 222. Once the osteotomies are performed, the resection interface 884 can include the resected surfaces on the distal end of the tibia 226 and a proximal end of the talus 22. Once the osteotomies are reduced, the resection interface 884 includes the resected surfaces abutting each other. Those of skill in the art will appreciate that for a given arthrodesis, ankle fusion procedure, the configuration of the osteotomies can be varied based on the experience level and/or desires of the surgeon in order to accomplish a successful fusion. Furthermore, the space between osteotomies and other structures of the ankle that a surgeon desires to preserve can be limited and thus require extra care. Advantageously, the present disclosure includes aspects to assist with accomplishing the desired osteotomies and preserving the surrounding structures. In one embodiment, these aspects can include one or more resection guards 870 and/or one or more stops. In certain embodiments, a resection guard 870 can serve as a stop.

In certain embodiments, the osteotomy system 800 may include one or more resection guards 870. A resection guard 870 is a structure, device, part, component, or apparatus designed and/or positioned to mitigate or prevent resection beyond a particular point or boundary. The resection guard 870 can and/or does help a surgeon limit resection within a certain area of the bone as guided by a resection feature 826 and resection guard 870. In the illustrated embodiment, fasteners 810 may cooperate with the resection features 826 and/or openings in the resection features 826 to serve as both fasteners and as resection guards 870. Alternatively, or in addition, other structures may be used to serve as resection guards 870. In one embodiment, the fasteners 810 and/or resection guards 870 can be strategically positioned in the resection guides 820 and/or configured (e.g., having a particular length) such that the fasteners 810 and/or resection guards 870 also retain or retract soft tissue around an opening formed for performing the osteotomy.

Advantageously, the resection guards 870 provide a boundary within the respective bones to prevent a cutting tool from drifting, or moving, outside an area defined by a resection feature 826. Resection of the talus 222 and/or tibia 226 can require deep cuts and keeping the cutting tool within the area defined by the resection feature 826 can be a challenge as the resection extends deeper and deeper into a bone. Advantageously, the resection guards 870 can assist the surgeon to avoid inadvertent resection of structures near or adjacent to the area indicated by the resection feature 826 (e.g., medial malleolus, lateral malleolus, etc.). In this manner, resection guards 870 can be a form of stop used in the osteotomy system 800.

As an osteotomy is performed in the resection feature 826 a surgeon may have limited to no information about how deep the osteotomy is extending into the bone. Of course, a surgeon can check depth using medical imaging such as fluoroscopy, however this can add time and expense to a procedure. In one embodiment, a preoperative plan included in the osteotomy system 800 can identify the width and length of cutting tool, such as a saw for a surgeon to use for each of the osteotomies. The saw may include laser etch depth markings on the blade such that as the blade enters a surgeon can read the depth from the blade. Alternatively, or in addition, the present disclosure can provide one or more stops to assist a surgeon in managing depth of the osteotomy.

In one embodiment, a stop 886 can be implemented in the form of a distance of a resection feature 826 between an opening on an anterior side 838, 852 and an opening on the posterior side 840, 854. In one embodiment, a portion of the body 836,850 may be of a predetermined length just around the resection features 826, Alternatively, or in addition, the whole body may have the predetermined length to provide the stop 886. Those of skill in the art will appreciate the one or the other or both of the resection feature 826 may include this type of stop.

Alternatively, or in addition, another structure may be formed at or near the resection feature 826 and serve as a stop 886. In this format, the distance within the resection feature 826 combined with a particular length for the blade or bur of the cutting tool may serve as a stop 886. Advantageously, a surgeon using such a configured resection guides 820 need not check or monitor the depth when forming the osteotomy. The surgeon can simply insert the cutting tool until the tool abuts the resection feature 826 of the resection guide 820 (“bottoms out”). At this point, the surgeon knows the cutting tool has reached a prescribed depth and that tissue beyond the depth is preserved.

In the illustrated embodiment, a resection guard 870 is positioned at each end of the resection feature 826 of the tibial resection guide 822 and at each end of the resection feature 826a of the resection guard 870. Of course, the resection guard 870 may be positioned at other points within, along, or near the resection features 826. Advantageously, because the resection guides 820 can be manufactured to meet a specific patient's anatomy, more precise placement and/or orientation of resection guards 870 can enable preservation of a joint of the patient and/or avoid resection of adjacent structure within the area of the joint. Those of skill in the art will appreciate that the use of one or more resection guards 870 may be optional and may depend on the user instructions 604 as to whether resection guards 870 are included, where they are positioned, oriented, and/or used.

Advantageously, the osteotomy system 800 can include a plurality of stops 886 and/or resection guards 870 that can assist a surgeon during the cutting in or between any of the planes of the foot and/or ankle. These stops 886 are configured to prevent a cutting tool from cutting tissue beyond a boundary defined at least partially using a bone model of at least a portion of a patient's foot and/or ankle. Those of skill in the art will appreciate that as an osteotomy is formed in one or both of the resection feature 826 and resection guards 870 are being used, the osteotomy will free the resection guard 870. For certain surgeons this may be undesirable. Advantageously, a surgeon can include in the user instructions 604 that specify whether or not to include stops 886 and/or resection guards 870, how many to include, where to position them, and the like. Thus, a surgeon can control how and where stops 886 may be used.

In one embodiment, the tibial resection guide 822 may include an alignment guide 874. The alignment guide 874 includes a superior end and one or more openings 876 (See FIG. 9B) near the superior end. An alignment guide 874 may be coupled to, connected to, and/or extend from a tibial resection guide 822. Alternatively, or in addition, An alignment guide 874 may be coupled to, connected to, and/or extend from a talus resection guide 824. The alignment guide 874 assists a surgeon in confirming that a resection guide 820 is in a desired position relative to other anatomical structures and/or axes of a patient. In one embodiment, the alignment guide 874 indicates an orientation of a resection guides 820 relative to a mechanical axis 880 of a tibia 226 of the patient.

A surgeon may use the alignment guide 874 by inserting a shaft 878, such as a K-wire, into, or through, an opening 876. The openings 876 and alignment guide 874 may be configured such that a K-wire 878 within the opening 876 extends superiorly along a superior-inferior axis and indicates the orientation and alignment of the tibial resection guide 822 relative to a long axis or a mechanical axis 880 of the tibia 226. A surgeon may compare this alignment with a desired alignment and/or position and/or the orientation and/or alignment of other bones of the patient. In this manner, a surgeon can confirm that tibial resection guide 822 is properly positioned.

In certain embodiments, the alignment guide 874 may have an opening on both ends such that the shaft 878 can extend in a dorsal/cephalad direction and in a plantar/caudal away from the alignment guide 874, the lower extension of the shaft 878 can further assist a surgeon in checking alignment. Alternatively, or in addition, the alignment guide 874 can include one or more second openings that extend perpendicular to the opening(s) 876. These second openings are horizontal to the shaft 878 and may be configured to accept a second shaft that is perpendicular to the shaft 878. This second shaft may extend medially and laterally and may cross over the shaft 878. In one embodiment, the alignment guide 874 includes an opening that extends from an anterior surface to a posterior surface and is centered on where the shaft 878 crosses over the second shaft. In this manner, the alignment guide 874 may form a kind of sight, much like a gunsight. In one embodiment, a surgeon may use this site and medical imaging such as with a C-arm (e.g., fluoroscopy) to check or confirm a position of a resection guides 820 intraoperatively.

FIG. 12C illustrates an alternative view of the surgical procedure similar to the stage in FIG. 12A. However, the tibia 226 and fibula 228 are transparent. FIG. 12C illustrates how a surgeon may use the shaft 878, alignment guide 874, and/or one or more of the resection guard 870 to check and/or confirm the position, placement, alignment, and/or trajectory that cuts using the resection guides 820 will have in the bones of the patient. In the illustrated embodiment, the mechanical axis 880 may be perpendicular to an anterior-posterior axis 888. In certain embodiments, the surgeon may choose to position and/or orient the resection guard 870 so that they can serve as alignment and/or positioning guides during the surgical procedure.

In certain cases, a surgeon may position the tibial resection guide 822 on the tibia 226 and secure it using the bone attachment feature(s) 828. Next, the surgeon may insert the shaft 878 into the alignment guide 874 and may use flouroscopy from an anterior view and/or a lateral view to confirm the shaft 878 is parallel in the frontal plane 264 and transverse plane 266. Alternatively, or in addition, the surgeon may deploy or stage (just insert one end into holes in the resection guides 820 so that the pins are held but do not enter the bone) and then use flouroscopy from a lateral view to check that the resection guard 870 are parallel with the anterior-posterior axis 888 in the sagittal plane 262. Since the tibial resection guide 822 was positioned and/or designed using a model of at least a portion of the foot and ankle of the patient, the surgeon can be assured that the tibial resection guide 822 is in the position desired. Of course, a similar process for checking and/or confirming can be done with the talus resection guide 824 as desired.

FIG. 12D illustrates resection guides, according to another embodiment. In the illustrated embodiment, the resection guides 820 can include, again either by default and/or due to user instructions 604, one or more fastener guides 890. The fastener guides 890 are structures that serve to assist, facilitate, mark, and/or indicate where to position and/or place fasteners that are used independently and/or as part of a fixation system. In certain embodiments, the fastener guides 890 may be implemented as spikes or tines on a bone facing surface of the resection guides 820 that provide an opening in the bone that can be used when the resection guides 820 is pounded against the bone using a mallet.

Advantageously, a surgeon can indicate in the user instructions 604 which fasteners and/or fixation systems, the surgeon plans to use. Accordingly, the tibial resection guide 822 and/or talus resection guide 824 can be designed and/or fabricated to support those fastener guides 890.

In the illustrated embodiment, the example resection guides 820 include fastener guides 890 that can be implemented as holes in the bodies of the tibial resection guide 822 and/or talus resection guide 824. These fastener guides 890 may be laid out on the resection guides 820 in a pattern that matches the one or more holes for a fastener such as one or more bone plates, one or more bone staple, one or more bone screws, or the like that a surgeon may desired to use for provisional or permanent fixation. In one embodiment, the fastener guides 890 mark where pilot holes or hole marks can be made in the bone. Alternatively, or in addition, a surgeon may deploy pins that can be used in a fixation system. In still other embodiments, a fastener guides 890 may serve to enable deployment of fixation fastener, such as a cannulated headless bone screw. Those of skill in the art will appreciate that the fastener guides 890 can take a variety of forms and configurations and leverage the positioning and placement of the resection guides 820.

Of course, the bone attachment features 828 and/or the resection guard 870 can also be strategically positioned to serve as pilot holes and/or guides for fasteners and/or components of a fixation system used when fixation is needed.

FIGS. 12A-12D illustrate one example of one type of osteotomy the osteotomy system 800 can be used to create during the surgical procedure. In the illustrated embodiment, a first osteotomy formed by the tibial resection guide 822 is a straight cut and a second osteotomy formed by the talus resection guide 824 is a straight cut with a straight medial connected cut. Advantageously, the surgeon, by way for example of the user instructions 604 can defined what shape, size, type, and/or trajectory of osteotomy they want the resection guide 820 to assist them in creating. Said another way, the surgeon can define the shape, size, length, and/or configuration of the resection interface 884.

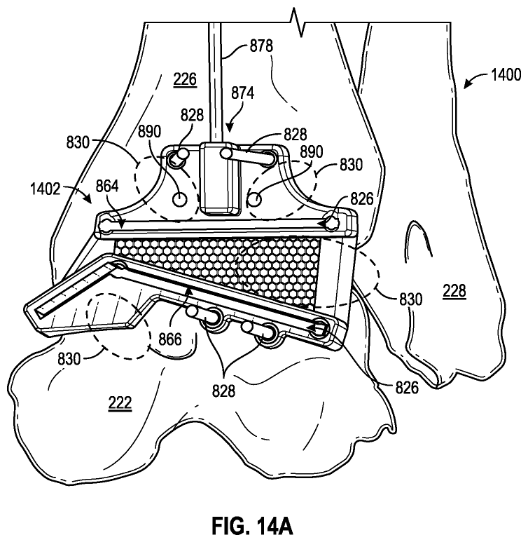

FIGS. 13A-13F illustrate alternative embodiments of a resection interface between two osteotomies using an exemplary osteotomy system, according to one embodiment. As described herein, the resection interface 884

Those of skill in the art will appreciate that the FIGS. 13A-13F illustrate examples and that other shapes and/or configurations can be provided within the scope of the present disclosure.

FIG. 13A illustrates the resection interface 884 of the example in FIGS. 12A-12D. As has been described, the resection interface 884 consists of a resected distal end 892 of the tibia 226 and a resected proximal end 894 of the talus 222. In certain embodiments, there are no other structures and/or implants between the resected distal end 892 and the resected proximal end 894. This is particularly the case where the surgical procedure is an ankle fusion.

The resection interface 884 shown in FIG. 13A, illustrates a view showing an anterior side of the bones and the resection interface 884 and the medial side is on the left and the lateral side is on the right. An anterior-posterior axis extends perpendicularly into and out of the view in FIG. 13A and the view illustrates a cross-section of the resection interface 884. FIG. 13A illustrates the cross-section of the resection interface 884 is a polygonal cross-section. In the illustrated embodiment, the polygonal cross-section includes two resected sides of the talus 222 and at least one side of the tibia 226. In FIG. 13A, the resected proximal end 894 of the talus 222 has an angled medial cut that connects to a straight cut. The angled medial cut may be implemented to enable the talus 222 to fit under the tibia 226 without interference from the medial malleolus when the osteotomies are reduced.

The resection interface 884 shown in FIG. 13B, illustrates a view showing an anterior side of the bones and the resection interface 884 and the medial side is on the left and the lateral side is on the right. An anterior-posterior axis extends perpendicularly into and out of the view in FIG. 13B and the view illustrates a cross-section of the resection interface 884. FIG. 13B illustrates that the cross-section of the resection interface 884 is a polygonal cross-section. In the illustrated embodiment, the polygonal cross-section includes two resected sides of the talus 222 and at least one side of the tibia 226.

In FIG. 13B, the resected proximal end 894 of the talus 222 has an angled medial cut that connects to a straight cut that connects to an angled lateral cut. The angled medial cut may be implemented to enable the talus 222 to fit under the tibia 226 without interference from the medial malleolus when the osteotomies are reduced. The angled lateral cut may be implemented to enable the talus 222 to fit under the tibia 226 without interference from the lateral malleolus when the osteotomies are reduced. Alternatively, or in addition, the resected distal end 892 of the tibia 226 may include an angled medial cut that connects to a straight cut that connects to an angled lateral cut. The angled medial cut, straight cut, and angled lateral cut of the tibia 226 can be configured to match and/or substantially match the angled medial cut, straight cut, and angled lateral cut of the talus 222. In this manner, the sides of the resection interface 884 may be configured to engage with each other.

The resection interface 884 shown in FIG. 13C illustrates a view showing an anterior side of the bones and the resection interface 884 and the medial side is on the left and the lateral side is on the right. An anterior-posterior axis extends perpendicularly into and out of the view in FIG. 13C and the view illustrates a cross-section of the resection interface 884. FIG. 13C illustrates that the cross-section of the resection interface 884 is a polygonal cross-section. In the illustrated embodiment, the polygonal cross-section includes five resected sides of the talus 222 and five resected sides of the tibia 226.

In FIG. 13C, the resected proximal end 894 of the talus 222 has five cuts that connect to each other at right (or substantially right) angles. Alternatively, or in addition, the resected distal end 892 of the tibia 226 may include five corresponding cuts that connect to each other at right (or substantially right) angles. The cuts of the talus 222 correspond to the tibia 226 such that an opening formed in the tibia 226 receives a shape formed in the talus 222. In this manner, the sides of the resection interface 884 may be configured to engage with each other.

Those of skill in the art will appreciate that the opening can be formed in the talus 222 and the shape that fits into the opening formed in the tibia 226. This can be done for example in response to user instructions 604. Similarly, those of skill in the art will appreciate that the shape of the cross section of the resection interface 884 can be any of a number of straight line and/or curved shapes. Furthermore, a resection interface 884 may be configured to have a keyhole and corresponding key shape configuration. Using FIG. 13C as an example, the opening formed by a first osteotomy of the resected distal end 892 of the tibia 226 can be referred to as a keyhole and the shaped extension formed in the resected proximal end 894 of the talus 222 can be considered a corresponding key configured to fit within the keyhole.

Of course, a variety of shapes for the resection interface 884 can be formed using the present disclosure. Like FIGS. 13A-13C, FIGS. 13D-13F illustrate alternative examples of resection interface 884 that illustrate a cross section for the resection interface 884 that can include straight side cuts, curved cuts, and/or a combination of these. In one embodiment, curved cuts may be formed for the osteotomies using a cutting tool such as a burr. FIG. 13D illustrates a triangular or chevron cross-section shape for the resection interface 884. FIG. 13E illustrates a single curved cross-section shape for the resection interface 884. FIG. 13F illustrates a multiple curve cross-section shape for the resection interface 884.

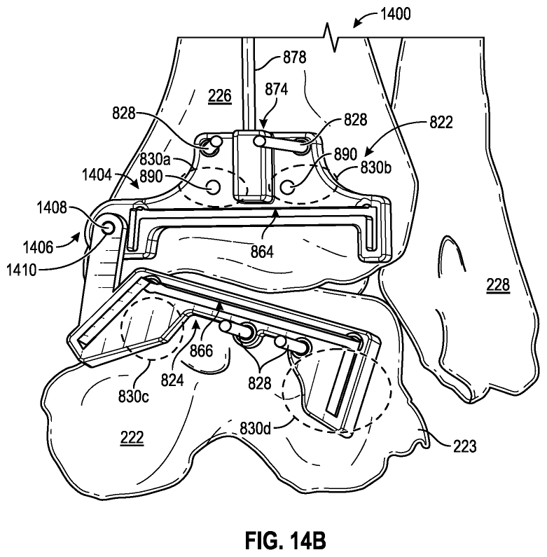

FIGS. 14A-14B illustrate views of resection guides of an osteotomy system 1400, according to alternative embodiments. FIG. 14A illustrates an alternative embodiment of a resection guide 820. The osteotomy system 1400 includes resection guide 1402.

The resection guide 1402 of osteotomy system 1400 can include some or all of the same or substantially the same features, aspects, and/or components as the resection guides 820 described herein with like components including the same reference numerals. Accordingly, the resection guide 1402 can or may include one or more tibia resection features 864, one or more talus resection feature 866, one or more bone attachment features 828, one or more bone engagement features 830, one or more alignment guides 874, one or more shafts 878, one or more openings 868, one or more resection guards 870, one or more fastener guides 890, and the like.

The resection guide 1402 may differ from other resection guides 820 described herein because the resection guide 1402 may include one or more tibia resection features 864 connected to one or more talus resection feature 866 by a single body. The body may extend from a distal end of the tibia 226 to the proximal end of the talus 222 across the ankle joint. In one embodiment, the single body may simply connect the tibia resection feature 864 and/or talus resection feature 866. Alternatively, or in addition, the single body may include a posterior side that includes a bone engagement surface configured to engage bones of the tibia and talus and engage with a space between the bones within the ankle joint. Advantageously, such a bone engagement surface can assist a surgeon in ensuring that that resection guide 1402 is properly positioned before being connected and/or attached to the bone(s). In one embodiment, the part of the body between the tibia resection feature 864 and the talus resection feature 866 can be a bone engagement feature 830.

As described herein, a surgeon can dictate whether the resection guides 820 is a single guide as in resection guide 1402, whether a bone engagement feature 830 is between the resection features 826, how many bone engagement features 830 to include, how many resection features to include, the configuration of the resection features, the configuration of the bone engagement features, and the like. A surgeon can also control whether the resection guide 1402 will include fastener guides 890 and/or an alignment guide 874. In one embodiment, a portion of the body that extends from a distal end of the tibia 226 to the proximal end of the talus 222 across the ankle joint (e.g., between the resection features 826) may also include a plurality of openings in a pattern. The openings may be six sided (i.e., hexagons). The openings may serve to facilitate visibility for a surgeon of the bones and/or space between the bones of the joint, particularly when the surgeon uses flouroscopy to confirm positioning and/or steps for a surgical procedure.

The resection guide 1402 can include a first resection feature 826 (e.g., tibia resection feature 864) coupled to a body of the resection guide 1402. The resection feature 826 can be configured to guide a cutting tool for form a first osteotomy in a first bone, such as a tibia 226. The first osteotomy is defined at least partially based on user directions and/or at least partially based on a bone model of at least a portion of the first bone (e.g., tibia 226). The bone model can be based on medical imaging of a patient's foot. Alternatively, or in addition, the resection guide 1402 can also include a second resection feature 826 (e.g., talus resection feature 866) coupled to a body of the resection guide 1402. The resection feature 826 can be configured to guide a cutting tool for form a second osteotomy in a second bone, such as a talus 222. The second osteotomy can be shaped to form a resection interface with the first osteotomy.

In addition, the resection guide 1402 can include a second bone attachment feature 828 in addition to a first attachment feature 828 of the resection guide 1402. The second bone attachment feature 828 can be configured to secure the resection guide 1402 to a second bone (e.g., talus 222). In one embodiment, the first bone and the second bone are part of a common joint. Securing the resection guide 1402 to a first bone (e.g., tibia 226) and a second bone (e.g., talus 222) can be assist a surgeon in performing the osteotomies just as outlined preoperatively, because the bone models used to prepare the resection guide 1402 and/or a preoperative plan may be dependent on each bone involved in the surgical procedure being in a predetermined position.

Advantageously, securing the resection guide 1402 to both a first bone with a first bone attachment feature 828 and a second bone with a second bone attachment feature 828 can ensure that the two bones are in the same position as the bone models used to design the resection guide 1402. This means that securing the two bones intraoperatively to the resection guide 1402 will also result in positioning and/or orienting the two bones for the osteotomies. Accurate bone positioning during the surgical procedure that corresponds to the bone positioning in a model (e.g., bone model(s) and/or instrument model(s)) ensures that angles, trajectories, sizes, and/or shapes designed into the instrument (e.g., resection guide 1402) will be used and leveraged during the surgical procedure.

FIG. 14B illustrates an alternative embodiment of a resection guide 820. The osteotomy system 1400 includes resection guide 1404. The resection guide 1404 can include some or all of the same or substantially the same features, aspects, and/or components as the resection guide 1402 described herein with like components including the same reference numerals. Accordingly, the resection guide 1404 can or may include one or more tibia resection features 864, one or more talus resection feature 866, one or more bone attachment features 828, one or more bone engagement features 830, one or more alignment guides 874, one or more shafts 878, one or more openings 868, one or more resection guards 870, one or more fastener guides 890, and the like.

The resection guide 1404 may differ from resection guide 1402 because the resection guide 1402 may include a tibial resection guide 822 that is or can be coupled to a talus resection guide 824 by a coupler 1406. In resection guide 1404 there is an open space between tibia resection feature 864 and talus resection feature 866. The coupler 1406 can be implemented in a variety of forms. In the illustrated embodiment, the coupler 1406 includes a pin of one of the tibial resection guide 822 and talus resection guide 824 that engages an opening in the other of the tibial resection guide 822 and talus resection guide 824. In the illustrated embodiment, the tibial resection guide 822 includes a pin 1408 that fits within an opening 1410 of the talus resection guide 824 to engage the coupler 1406. A surgeon may couple the tibial resection guide 822 preoperatively or intraoperatively.

As with the resection guide 1402 the resection guide 1404 may include a plurality of bone engagement features 830a, 830b, 830c. Alternatively, or in addition, certain bone engagement features 830 may be omitted from a particular resection guide 1404 based on user instructions 604.

In one embodiment, the inclusion of these bone engagement features 830 and/or the configuration (i.e., size, shape, etc.) of the bone engagement feature 830 can be based at least partially user instructions 604 and at least partially on a bone model of at least a portion of one or more a tibia 226 and/or a talus 222 of a patient. In one embodiment, the bone model may influence a configuration of a bone engagement surface(s) of the resection guide 1404. Alternatively, or in addition, certain aspects of the bone engagement features 830 may be based on a default configuration provided for the osteotomy system 1400.

In one embodiment, the bone engagement feature 830 is configured to engage with at least a portion of a first bone at a position that substantially matches a model position (i.e., model's position) of a model of the resection guide engaging a bone model of the first bone. In the illustrated embodiment, bone engagement features 830 of the tibial resection guide 822 and/or the talus resection guide 824 can include a bone engagement surface that matches, at least partially matches, and/or substantially matches, a surface of the bones the respective guides will contact when the guides are positioned for use.

Advantageously, the bone engagement surface have been generated based on models of at least a portion of the tibial resection guide 822 and/or talus resection guide 824 positioned on, or in relation to, models of one or more of the tibia 226 and talus 222. Because the bone engagement surface matches, or substantially matches, a contour of the surfaces of the bones, the bone engagement surface, when used, readily moves into the same position on the bone(s) as the model of the bone engagement surface had in relation to the models of the bones.

In certain embodiments, the bone engagement feature 830 includes a bone engagement surface and a body section, the body section extends from the body and supports the bone engagement surface. In certain embodiments, where a bone engagement feature 830 is unneeded or undesired, a body section of a resection guides 820 may be replaced with an opening or a void. Said another way, the body (or a body section) may not be formed/fabricated in an area of a resection guides 820 where a bone engagement feature 830 is not needed or desired.

FIG. 14B illustrates that a surgeon has provided user instructions 604 for including an additional bone engagement feature 830d. Specifically, the surgeon has requested that the body be extended on the lateral side, that the body include a bone engagement surface on its posterior side to engage bone of the talus 222 and that the talus resection feature 866 be configured to include a slot that extends in a plantar direction on the lateral side. In this embodiment, the surgeon may have requested the added bone engagement feature 830d to facilitate placement of the talus resection guide 824 on the talus 222. Alternatively, or in addition, the added bone engagement feature 830d may have been included to facilitate an osteotomy of the lateral side of the talus 222. For example, a surgeon may decide to resect a lateral extending process 223 of the talus 222. Advantageously, a surgeon can use embodiments of the present disclosure to obtain a resection guide 1404 that is patient-specific and customized to the needs and/or desires of the surgeon in order to provide improved patient care.

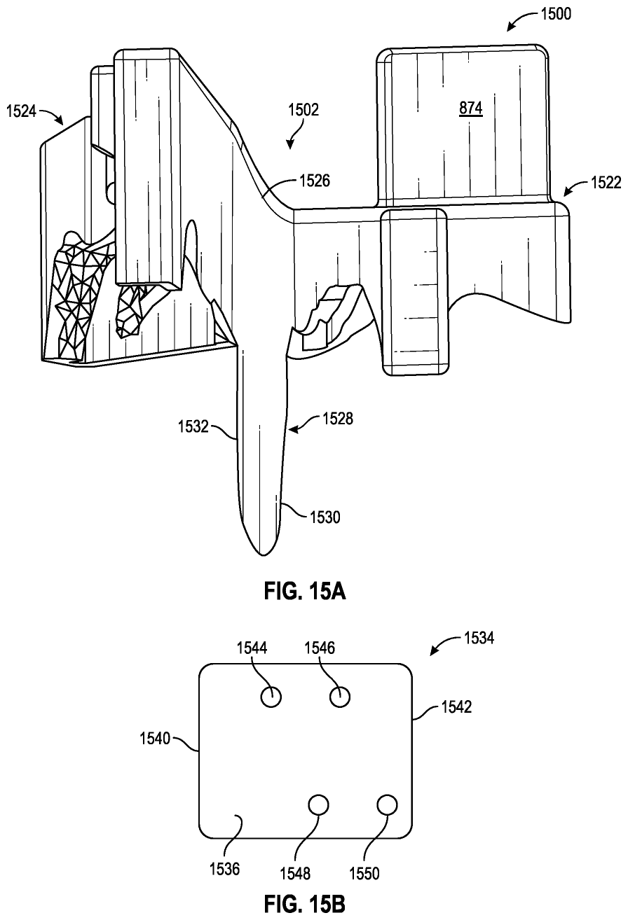

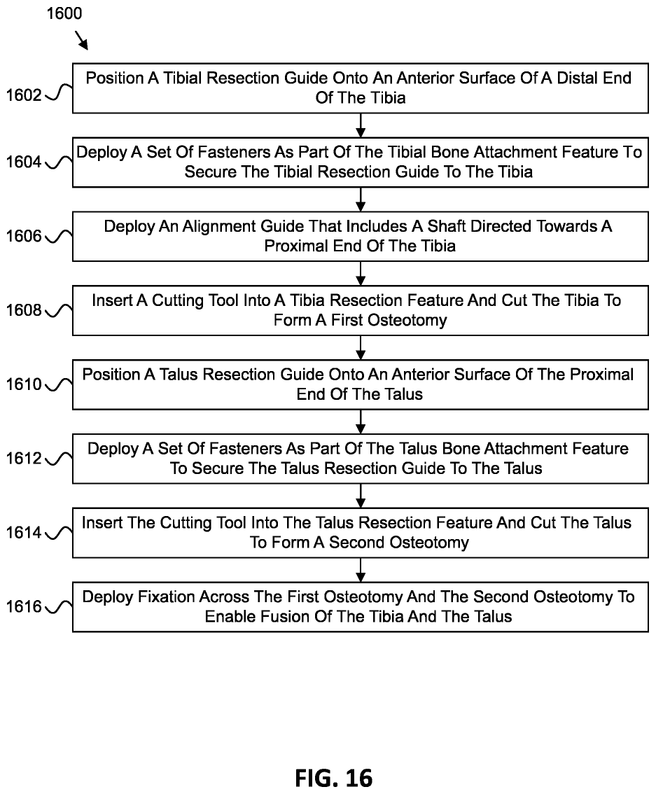

FIG. 15A illustrates a lateral side view of a resection guide 1502 of an osteotomy system 1500, according to one embodiment. The resection guide 1502 of the osteotomy system 1500 can include some or all of the same or substantially the same features, aspects, and/or components as the resection guides 820 described herein with like components including the same reference numerals. Accordingly, the resection guide 1502 can or may include one or more tibia resection features 864, one or more talus resection feature 866, one or more bone attachment features 828, one or more bone engagement features 830, one or more landmark registration features 834, one or more alignment guides 874, one or more shafts 878, one or more openings 868, one or more resection guards 870, one or more fastener guides 890, and the like.

In one embodiment, the resection guide 1502 can be designed and/or fabricated by combining a tibial resection guide 822 with a talus resection guide 824 by adding more body structure that connects and joins the two resection guides 820. Thus, the resection guide 1502, in one embodiment, can include a tibial resection guide 1522 connected to a talus resection guide 1524 and an interconnecting body section 1526.

FIG. 15A illustrates a lateral side view of the resection guide 1502. Those of skill in the art will appreciate that the tibial resection guide 1522 can be configured the same as or similarly to the tibial resection guide 822, including one or more bone attachment features 828 and/or one or more bone engagement surface 832 and/or one or more landmark registration features 834. Alternatively, or in addition, the talus resection guide 1524 can be configured the same as or similarly to the talus resection guide 824, including one or more bone attachment features 828 and/or one or more bone engagement surface 832 and/or one or more landmark registration features 834.

In addition, the resection guide 1502 can include a bone probe 1528. In one embodiment, the bone probe 1528 is a structure that extends away from the interconnecting body section 1526 from a posterior side of the resection guide 1502. In one embodiment, the bone probe 1528 is configured to extend such that when the resection guide 1502 is in use the bone probe 1528 extends into a joint between a first bone (e.g., tibia 226) and a second bone (e.g., talus 222). In one embodiment, the bone probe 1528 is configured to engage, at least partially, with a landmark associated with one or the other or both of the first bone and the second bone. In one embodiment, the landmark of one or both of the bones may be a protrusion, a projection, a tuberosity, a cavity, a void, a divot, a tab, an extension, a hook, a curve, or the like of one or both of the bones and/or a structure on a surface of one or both of the bones. In one embodiment, a distal end of the bone probe 1528 can be patient-specific and configured to substantially match a surface of one or the other of the bones of a joint.

In one embodiment, the bone probe 1528 starts with a first width and tapers to a narrow width moving from a proximal end of the bone probe 1528 to a distal end of the bone probe 1528. In one embodiment, the bone probe 1528 can have a width that is similar to the width of the resection guide 1502 from medial to lateral. Alternatively, or in addition, the bone probe 1528 can have a width that is smaller than the width of the resection guide 1502 from medial to lateral. In another embodiment, the bone probe 1528 can have a width that is greater than the width of the resection guide 1502 from medial to lateral.

In one embodiment, the bone probe 1528 can have a superior surface 1530 and an inferior surface 1532. In one embodiment, one or the other or both of the superior surface 1530 and the inferior surface 1532 can include a bone engagement surface 832. In one embodiment, the bone engagement surface 832 can be configured to match and/or substantially match a corresponding surface of the first bone (e.g., tibia 226) and/or the second bone (e.g., talus 222).

Those of skill in the art will appreciate that just as the resection guide 1502 can be designed to include a bone probe 1528, a body of the tibial resection guide 1522, the talus resection guide 1524, and/or a resection guide 1402 can include additional features that extend anteriorly from an anterior surface (the one facing away from the bone(s) when the device is in use with an anterior approach surgical procedure). These additional features can be referred to as resection guards or resection retractors and may serve to retain soft tissue around the edges of one or more incisions for the surgical procedure. In particular, these resection guards or resection retractors can be configured to extend anteriorly from the resection guide 1502 and of a distance sufficient to meet or exceed a skin surface for the patient during the surgical procedure. The resection guards or resection retractors may keep soft tissue from moving to close the incision(s) and obscuring a view for a surgeon and/or interfering with any cutting tools or other instruments during a surgical procedure.

FIG. 15B illustrates an anterior view of a positioning guide 1534 of an osteotomy system, according to one embodiment. In one embodiment, the osteotomy system 800 include one or more guide anchors. The positioning guide 1534 may use the guide anchors.

A guide anchor is an anchor for a resection guide 820 (e.g., tibial resection guide 822, talus resection guide 824, resection guide 1402, resection guide 1502, etc.). The guide anchor serves to hold a resection guide in place during an osteotomy. In one embodiment, the guide anchor is implemented by way of a pin (e.g., fastener) and an opening in the resection guide 820. In the illustrated embodiment, the pins may be the pins of the bone attachment feature 828 and/or the pins of the resection guard 870 and/or the pins of the fastener guides 890.

In one embodiment, the pins of the fastener guides 890 may enter the tibia 226 in parallel to each other which enables the tibial resection guide 822 to be slid off of the pins of the fastener guides 890 once an osteotomy of the tibia 226 is completed with the pins remaining in the tibia 226. These pins may be referred to as tibia pins. Similarly, the pins of the bone attachment features 828 of the talus resection guide 824 may be deployed parallel to each other and may remain when the talus resection guide 824 is removed. These pins may be referred to as talus pins. The tibia pins and/or talus pins may be referred to as reference features.

Advantageously, because the surgical procedure is preplanned using medical imaging of bones of a patient the same pins that serve as reference features can also serve as guide anchors. Those of skill in the art will appreciate that a reference feature can serve as an alignment feature or a resection guide anchor or both depending on the embodiment.

The positioning guide 1534 engages the guide anchors, reference features (e.g., tibia pins and/or talus pins) to position the talus 222 relative to the tibia 226 for a reduction of osteotomies of each of these bones. Alternatively, or in addition, the positioning guide 1534 holds the reduction closed and stable once the positioning guide 1534 has been slid to contact one of the other or both of the bones.

The positioning guide 1534 includes an anterior side 1536, posterior side 1538 (not shown), a medial side 1540, and a lateral side 1542. The positioning guide 1534 also includes a first proximal opening 1544, a second proximal opening 1546, first distal opening 1548, and a second distal opening 1550.

In one embodiment, a surgeon uses the positioning guide 1534 after a first osteotomy of a first bone and a second osteotomy of a second bone. In one embodiment, the two bones are a tibia 226 and a talus 222 and the positioning guide 1534 is configured to have a size that extends along a superior-inferior axis across the resection interface, the reduced osteotomies. In one embodiment, a surgeon has retained the tibia pins in the tibia 226 and the talus pins in the talus 222. These pins may come from a tibial bone attachment feature and a talus bone attachment feature. The positioning guide 1534 is configured to cooperate with one of the tibial bone attachment feature and the talus bone attachment feature to abut a first osteotomy against a second osteotomy in a stable relationship to close the resection interface.

At this stage, a surgeon is prepared to reduce the osteotomies and prepare for provisional and/or permanent fixation. Next, a surgeon slips the first proximal opening 1544 and the second proximal opening 1546 over the tibia pins and the first distal opening 1548 and second distal opening 1550. Next, the surgeon slides the positioning guide 1534 towards the bones. As the positioning guide 1534 slides towards the bones, the osteotomies are reduced and the two resected surface of the bones are brought together. In one embodiment, the talus 222 may be translated toward the tibia 226 until the talus 222 abuts the tibia 226. At this stage, the positioning guide 1534 may be in contact with or near the surface of the bones. The positioning guide 1534 is in its final position and the resection interface is closed. A surgeon can now prepare for a subsequent step such as fixation.

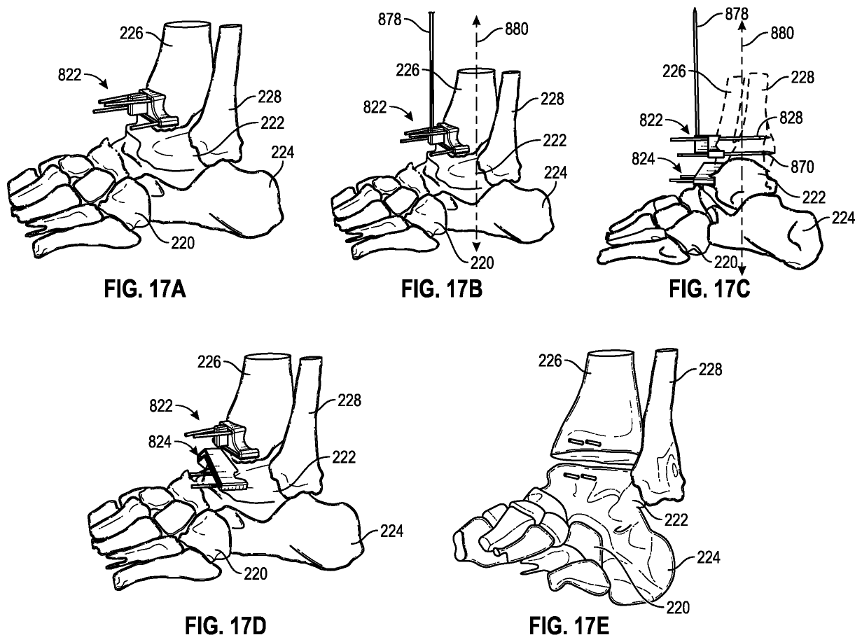

FIG. 16 is a flowchart diagram depicting a method for remediating a bone condition, according to one embodiment. FIG. 16 is a flowchart of an example method 1600. In some implementations, one or more method steps of FIG. 16 may be performed by a surgeon using one or more of the apparatuses, systems, and/or components described here.

As shown in FIG. 16, method 1600 may include positioning a tibial resection guide onto an anterior surface of a distal end of the tibia. The tibial resection guide includes a body having an anterior side, a posterior side, a medial side, a lateral side, a superior side, and an inferior side; a tibia resection feature configured to guide a cutting tool to prepare the tibia for fusion to a talus; a tibial bone attachment feature configured to secure the tibial resection guide to the tibia; a bone engagement feature having a bone engagement surface configured to at least partially match a contour of a portion of the anterior surface of the distal end of the tibia when the tibial resection guide is positioned for use. The tibial resection guide is defined based at least partially on user directions and at least partially on a bone model of at least a portion of the tibia, the bone model based on medical imaging of a patient's foot (block 1602). For example, a surgeon may position a tibial resection guide onto an anterior surface of a distal end of the tibia, as described above.

As also shown in FIG. 16, method 1600 may include deploying a set of fasteners as part of the tibial bone attachment feature to secure the tibial resection guide to the tibia (block 1604).

As further shown in FIG. 16, method 1600 may include deploying an alignment guide that includes a shaft directed towards a proximal end of the tibia (block 1606).

As also shown in FIG. 16, method 1600 may include inserting the cutting tool into the tibia resection feature and cutting the tibia to form a first osteotomy (block 1608).

As further shown in FIG. 16, method 1600 may include positioning a talus resection guide onto an anterior surface of the proximal end of the talus. The talus resection guide includes: a body having an anterior side, a posterior side, a medial side, a lateral side, a superior side, and an inferior side; a talus resection feature configured to guide the cutting tool to prepare the talus for fusion to the tibia; a talus bone attachment feature configured to secure the talus resection guide to the talus; a bone engagement feature having a bone engagement surface configured to at least partially match a contour of a portion of the anterior surface of the proximal end of the talus when the talus resection guide is positioned for use; where the talus resection guide is defined based at least partially on user directions and at least partially on a bone model of at least a portion of the talus, the bone model based on medical imaging of the patient's foot (block 1610). For example, a surgeon may position a talus resection guide onto an anterior surface of the proximal end of the talus, as described above.

As also shown in FIG. 16, method 1600 may include deploying a set of fasteners as part of the talus bone attachment feature to secure the talus resection guide to the talus (block 1612).

As further shown in FIG. 16, method 1600 may include inserting the cutting tool into the talus resection feature and cutting the talus to form a second osteotomy (block 1614).

As also shown in FIG. 16, method 1600 may include deploying fixation across the first osteotomy and the second osteotomy to enable fusion of the tibia and the talus (block 1616).

Method 1600 may include additional implementations, such as any single implementation or any combination of implementations described herein and/or in connection with one or more other methods or processes described elsewhere herein. A first implementation, method 1600 further includes accessing an anterior surface of a distal end of a tibia and an anterior surface of a proximal end of a talus of a patient's foot; deploying a set of stops within the tibia resection feature to manage the cutting tool; verifying the position of the tibial resection guide by comparing the shaft to a mechanical axis of the tibia and a set of fasteners deployed using the tibial resection guide; and reducing the first osteotomy and the second osteotomy by abutting the resected distal end of the tibia and the resected proximal end of the talus. When verifying the position, the surgeon may leverage one or more shafts 878 positioned in one or more alignment guides 874, one or more bone attachment features 828 (i.e., pins in bone), and/or one or more resection guards 870 (e.g., reference features) that may be configured to have a trajectory and/or orientation relative to axes of bones of the patient's foot such that a surgeon can used these reference features to verify that instruments are desired positions.

Although FIG. 16 shows example blocks or steps of a method 1600, in some implementations, a method 1600 may include additional steps, fewer steps, different steps, or differently arranged steps than those depicted in FIG. 16. Additionally, or alternatively, two or more of the steps of a method 1600 may be performed in parallel.

In one embodiment, the present disclosure can include an alternative method to the method 1600 and/or a separate method which includes: obtaining a prescription for an osteotomy system and/or for a component of an osteotomy system. The alternative method may include modifying an instrument model of an instrument to be included in the osteotomy system. In one embodiment, the modifications to the instrument model may be made based on or due to or at least in response to a received prescription. Next the alternative method, may include sending a report, a message, a notification, a set of models that includes a bone model and/or a model of one or more instruments of the osteotomy system, or the like to a surgeon who issued the prescription for review by the surgeon. The surgeon may then provide feedback in response to the report, a message, a notification, a set of models that includes a bone model and/or a model of one or more instruments of the osteotomy system, or the like. Optionally, the alternative method may include implementing any feedback provided by the surgeon. Of course, the method could iterate with more notices of changes to the surgeon and feedback provided by the surgeon, until a satisfactory design (i.e., approved by the surgeon) is generated. Next, the alternative method could conclude by arranging for and/or fabricating the osteotomy system and/or components of the osteotomy system.

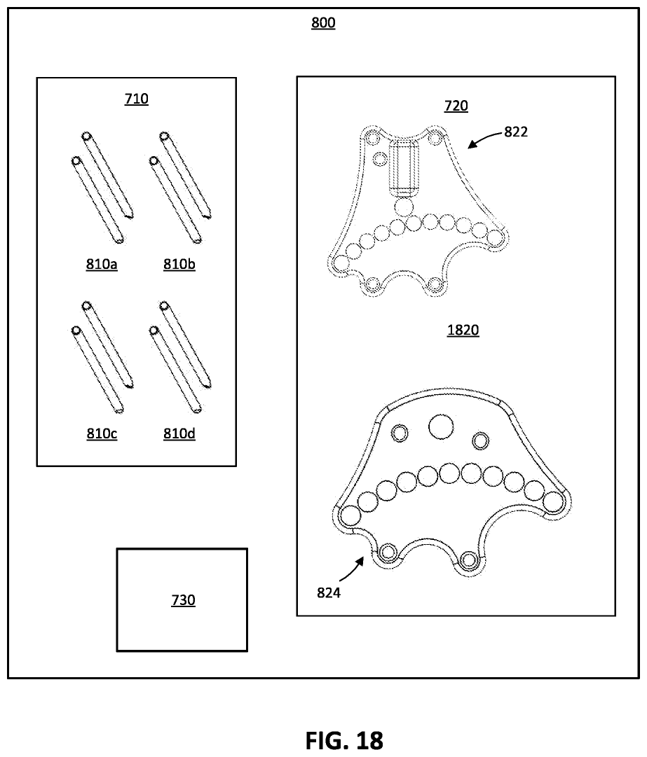

FIGS. 17A-17E illustrate different views of one or more stages in a surgical procedure that includes one or more embodiments of the present disclosure. Reference is made to FIGS. 8-10F, 12A-12D and FIG. 16.

FIG. 17A illustrates a stage of the method 1600 in which a tibial resection guide 822 is positioned 1602 on an anterior surface of a distal end of a tibia 226. In one embodiment, the tibial resection guide 822 and/or talus resection guide 824 may be made of metal such that they have a greater mass than resection guides 820 fabricated from materials having a lower mass such as polymers and/or ceramics. Using a resection guide 820 with a greater mass can facilitate registering the resection guide 820 to a bone surface during positioning steps.

In the illustrated embodiment, the surgeon has made an incision that permits access to an anterior surface of a distal end of a tibia and an anterior surface of a proximal end of a talus of a patient's foot. Pins of two bone attachment features 828 (e.g., one or more tibial bone attachment features) have also been deployed to secure the tibial resection guide 822 to the tibia 226. Note that the tibial resection guide 822 includes an alignment guide 874, but the shaft 878 has not yet been inserted.

FIG. 17B illustrates a stage in the surgical procedure in which the shaft 878 is inserted in the alignment guide 874. A surgeon has deployed the alignment guide 874 according to one embodiment. A surgeon can now visually check manually, and/or with flouroscopy, a position of the tibial resection guide 822 in relation to a mechanical axis 880 of the tibia 226. In the illustrated embodiment, the surgeon has also deployed a plurality of resection guards 870.

FIG. 17C illustrates a lateral view with the fibula 228 and tibia 226 transparent. This illustrates a view a surgeon can take using the resection guards 870 and/or shafts 878 in relation to the mechanical axis 880. Advantageously, a surgeon can check the position, alignment, and/or trajectories in all three planes before performing a first osteotomy. In this manner, the surgeon can confirm that the osteotomy will be where intended and planned.

In one embodiment, FIG. 17D illustrates the ankle joint after method step 1610 is completed. Those of skill in the art will appreciate that the surgeon may opt to deploy the talus resection guide 824 prior to forming a first osteotomy using a cutting tool. Alternatively, or in addition, the surgeon may insert a cutting tool into a tibia resection feature 864 and cut the tibia 226 to form a first osteotomy prior to positioning a talus resection guide 824.

Once the example talus resection guide 824 is positioned, a surgeon may deploy a set of fasteners/pins as part of one or more bone attachment feature 828 (e.g., one or more talus bone attachment features) to secure the example talus resection guide 824 to the talus 222.

Next a surgeon may operate the example talus resection guide 824 by inserting 1614 a cutting tool such as a rectangular oscillating blade or a burr attached to a manual, mechanical, pneumatic, or electric driver into a resection feature to cut the talus 222 for an osteotomy. If the surgeon has not yet formed a first osteotomy using the tibial resection guide 822, the surgeon may also insert 1608 a cutting tool (possibly the same cutting tool as used for the tibial resection) into a resection feature of the tibial resection guide 822 to form a first osteotomy.

In one embodiment, FIG. 17E illustrates a stage in which a tibial resection guide 822 and a talus resection guide 824 have been removed. Pins in the tibia 226 may be retained and pins in the talus 222 may be retained. A first osteotomy has been formed at the distal end of the tibia 226 and a second osteotomy has been formed at the proximal end of the talus 222. A resection interface that includes the two osteotomies can now be closed. Once closed, and/or the osteotomies are reduced, a surgeon can proceed with fixation across the first osteotomy and the second osteotomy to enable fusion for the tibia 226 to the talus 222. In one embodiment, a surgeon may deploy a positioning guide 1534 to assist with the reduction and/or to hold the reduction in a stable relationship while fixation is deployed.

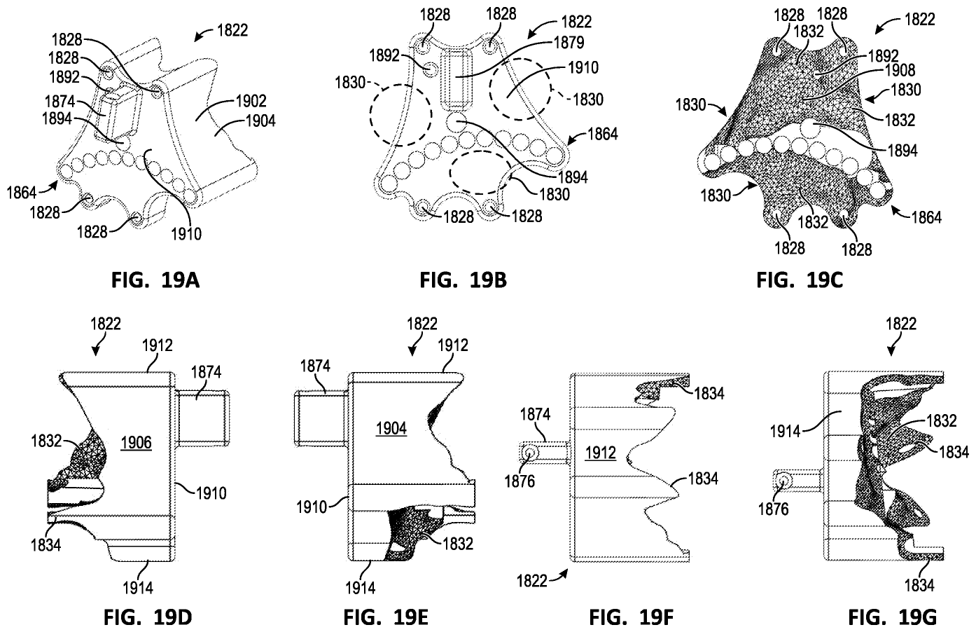

FIG. 18 illustrates an exemplary ankle fusion osteotomy system 1800, according to one embodiment. In one embodiment, the exemplary ankle fusion osteotomy system 1800 is specifically designed for use on a lateral side of the patient's ankle (e.g., using a lateral approach). Those of skill in the art will appreciate that the same or a similar exemplary ankle fusion osteotomy system 1800 can be used on a medial side of the patient's ankle (e.g., using a medial approach). Those of skill in the art will also appreciate that a surgical procedure that conducts an ankle fusion on an ankle from a medial or lateral approach generally includes steps to perform an osteotomy on the malleolus (medial or lateral depending on the approach) and retracting or holding a distal end of the malleolus out of the way for the osteotomy steps on the tibia 226 and/or talus 222. Those of skill in the art will appreciate that the osteotomy system 1800 can include one or more components that are patient-specific and/or one or more components that are not patient-specific. Thus, the osteotomy system 1800 can be a combination of patient-specific components and/or non-patient-specific components.

The osteotomy system 1800 can include one or more fasteners 810 and/or two or more sets of fasteners (810a, 810b, 810c), one or more resection guides 1820, and zero or one or more complementary components 730. In the illustrated embodiment, the resection guides 1820 include a first resection guide 1822 and a second resection guide 1824. The resection guides 1820 may also include one or more of resection features 1826, bone attachment features 1828, bone engagement feature 1830, and/or landmark registration feature 1834. In certain embodiments, a bone engagement feature 1830 can include a bone engagement surface 1832.

While specific embodiments of complementary components 730 are not specifically shown here in relation to the osteotomy system 1800, those of skill in the art will appreciate that complementary components 730 can be similar in feature, design, implementation, configuration, and purpose as those described in relation to the osteotomy system 700 and can be used for the osteotomy system 1800. Thus, the osteotomy system 1800 can include one or more alignment guides 740, rotation guides 750, correction guides 760, compression guides 770, positioning guides 780, fixation guides 790, navigation guides 792, implants 794, or the like.

Either or both of the resection guides 1820 may be custom patient-specific resection guides made for a particular patient and/or for a particular surgical procedure. Various aspects of the resection guides 1820 may be patient-specific, including, but not limited to, an angle and/or orientation for a resection feature of the resection guide 1820, a position of the resection feature, a depth of the resection feature, a size of the resection guide 1820, a configuration and/or composition of a bone contacting surface such as a bone engagement surface of the resection guide 1820, and the like.

Either or both of the resection guides 1820 of osteotomy system 1800 can include some or all of the same or substantially the same features, aspects, alternatives, and/or components as other example resection guides described herein with like components including the same or similar reference numerals. Accordingly, the resection guide 1822 can or may include a first resection feature 1864, a second resection feature 1866, one or more bone attachment features 1828, one or more bone engagement features 1830, one or more alignment guides 1874, one or more shafts 1878, one or more openings 1876, one or more resection guards 1870 (not shown), one or more fastener guides 1890 (not shown), and the like. The bone engagement features 1830 may include one or more bone engagement surfaces 1832. Alternatively, or in addition, either or both of the resection guides 1820 can include one or more stabilizers 1892 and one or more sights 1894.

The resection guides 1822 may each include a body that includes an anterior side, a posterior side, a medial side, a lateral side, a superior side, and an inferior side. In certain embodiments, a first resection guide 1822 may be combined with a second resection guide 1824 into a single resection guide 1820.

In one embodiment, the resection guides 1820 may differ from other resection guides described herein because the first resection feature 1864 and/or the second resection feature 1866 may differ from other example resection features described herein.

In the illustrated embodiment, the first resection feature 1864 and the second resection feature 1866 are configured to form one or more osteotomies for a “dome” osteotomy. A dome osteotomy is a form of osteotomy that results in resected bone surface resembling or taking the shape of a dome or hemisphere. In the illustrated embodiment, the dome osteotomy can be accomplished by a resection feature that forms a curved osteotomy. In certain embodiments, a curved osteotomy can be a challenge for form using a cutting tool such as a reciprocating saw blade. However, where the cutting tool is a burr, a curved osteotomy is easier to form.

In the illustrated embodiment, the first resection feature 1864 and second resection feature 1866 are configured to facilitate forming a curved osteotomy (and/or an angled curved osteotomy). Specifically, in one embodiment, the first resection feature 1864 and/or second resection feature 1866 may include a plurality of openings arranged in a curved pattern. Each of the openings may be sized to accept a cutting tool such as a burr. The burr drills a hole in bone opposite the opening. Alternatively, or in addition, the burr can be angled to resect bone between the openings. By forming bone tunnels or holes using the plurality of openings a surgeon can form a curved osteotomy (and/or an angled curved osteotomy) in the bone. Furthermore, angling the burr once in the bone can enable a surgeon to interconnect adjacent bone tunnels to complete the osteotomy.

In another embodiment, a resection feature (e.g., first resection feature 1864 and/or second resection feature 1866) can be implemented by way of a slot that has a curve, or curved, shape. The resection feature may extend into a body of a resection guide 1820 from one side and out an opposite side and the resection feature includes a first end and a second end. The resection feature may have a curve or curved shape between the first end and the second end. Such a resection feature is referred to herein as a curved slot. In one embodiment, the curved slot may have a curved cross section similar to the curve shape illustrated in the example first resection feature 1864 and/or second resection feature 1866. In one embodiment, a surgeon may insert a cutting tool such as a burr into bone through a curved slot and follow a path between the first end and the second end of the curved slot to resect a curved osteotomy.

Alternatively, or in addition, a single resection guide 1820 may include a pair of resection features. A first resection feature can include a plurality of openings in a curved shape with a few millimeters between them along the arc of the curve. A surgeon may first form holes in bone using each of the openings of the first resection feature. Next, a surgeon may replace the first resection feature with a second resection feature. The second resection feature may include a plurality of openings that are offset from the holes of the first resection feature. The offset holes enable a surgeon to form holes or tunnels in the bone that are adjacent to and may interconnect with the holes formed using the first resection feature. In this manner, a surgeon can accurately form a curved osteotomy (and/or an angled curved osteotomy) in the bone. In one embodiment, the first resection feature and second resection feature may be interchangeable by way of a coupler.

Referring now to FIGS. 2E, 18, and 19A-G, a first resection guide 1822 includes a first resection feature 1864. The first resection feature 1864 is configured to guide a cutting tool for form a curved osteotomy. The curved osteotomy is curved when viewed and/or measured within a sagittal plane 262 of an ankle of a patient. In particular, the curved osteotomy is formed by a pattern of a plurality of openings arranged in a curve within the body of the first resection guide 1822. In the illustrated embodiment, the pattern is a downward curve shape. Where the first resection guide 1822 is used on a proximal end of a bone, this pattern results in a convex curve shape on the end of the bone resected using the first resection guide 1822.

The curved osteotomy is determined, at least partially, based on a bone model of at least a portion of the patient's ankle. The bone model is based on medical imaging of the patient's ankle. A curve osteotomy is an osteotomy that has a curve shape that starts on one end and includes at least one arc and ends at another end.

In the illustrated embodiment, by forming a bone tunnel in the bone aligned with each of the plurality of openings in the first resection feature 1864, and then interconnecting the bone tunnels, a curved shape (like a dome) can be formed on an end or within a bone of a patient. The bone tunnels can be interconnected by angling a burr cutting tool within one opening in one direction and/or the other (e.g., medially and/or laterally). In one embodiment, the openings of the plurality of openings have a diameter that permits a burr cutting tool to be moved in a way to interconnect bone tunnels. Alternatively, or in addition, the bone tunnels can be interconnected by using an osteotome to remove walls between adjacent bone tunnels.

Referring now to FIGS. 2E, 18, and 20A-G, in the illustrated embodiment, a second resection guide 1824 includes a second resection feature 1866. The second resection guide 1824 is configured to guide a cutting tool to form an angled curved osteotomy. The angled curved osteotomy is similar to a curved osteotomy in that the angled curved osteotomy is curved when viewed and/or measured within a sagittal plane 262 of an ankle of a patient.

In addition, an angled curved osteotomy extends into a bone at an angle that is not perpendicular to a long axis (e.g., mechanical axis 880 of a bone). In the illustrated embodiment, the angled curved osteotomy may extend into a tibia at first angle 1896 that is not perpendicular to a long axis of the tibia 226 (e.g., mechanical axis 880 of the tibia 226). In one embodiment, the first angle 1896 is measured within a frontal plane 264. In particular, the first angle 1896 can be measured in relation to a horizontal medial-lateral axis ML within the frontal plane 264. In certain embodiments, the first angle 1896 can range from 1 degree to 88 degrees and can be extend from the ML axis in either a dorsal direction or a plantar direction.

In particular, the curved osteotomy is formed by a pattern of a plurality of openings arranged in a curve within the body of the second resection guide 1824. In the illustrated embodiment, the pattern is a downward curve shape.

The curved osteotomy is determined, at least partially, based on a bone model of at least a portion of the patient's ankle. The bone model is based on medical imaging of the patient's ankle. Those of skill in the art will appreciate that a curved osteotomy and an angled curved osteotomy are formed by a first resection feature 1864 and/or a second resection feature 1866 having a trajectory in relation to the a bone to be resected that matches or substantially matches the angle that the curved osteotomy and/or angled curved osteotomy will have once a cutting tool is used to form the curved osteotomy and/or angled curved osteotomy by way of the first resection feature 1864 and/or a second resection feature 1866.

In the illustrated embodiment, by forming a bone tunnel in the bone aligned with each of the plurality of openings in the second resection feature 1866, and then interconnecting the bone tunnels, a curved shape (like a dome) can be formed on an end or within a bone of a patient. The bone tunnels can be interconnected by angling a burr cutting tool within one opening in one direction and/or the other (e.g., medially and/or laterally). In one embodiment, the openings of the plurality of openings have a diameter that permits a burr cutting tool to be moved in a way to interconnect bone tunnels. Alternatively, or in addition, the bone tunnels can be interconnected by using an osteotome to remove walls between adjacent bone tunnels.

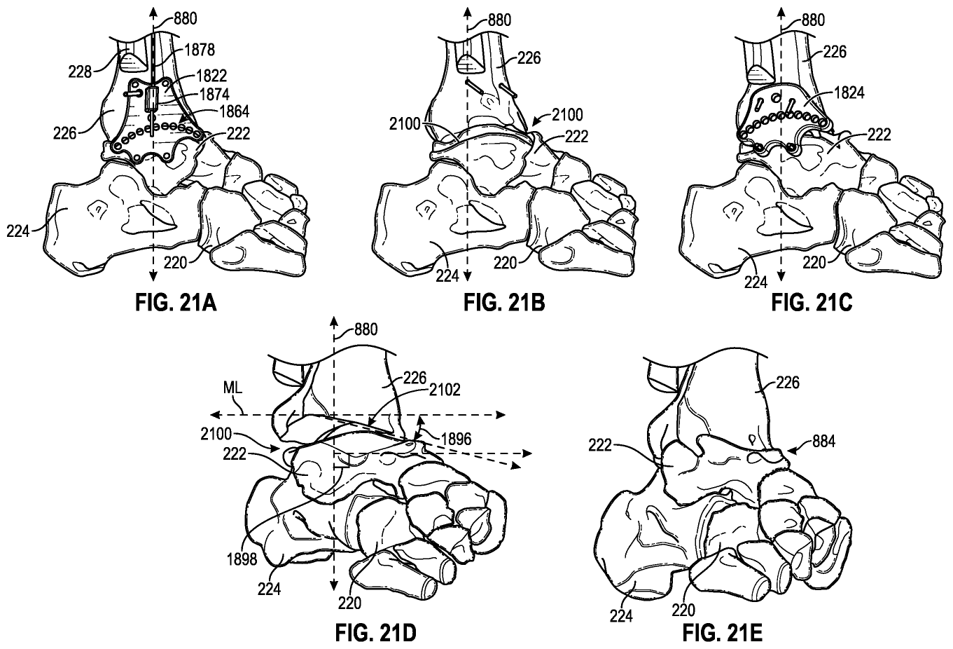

In the illustrated embodiment, a curved osteotomy may refer to an osteotomy formed using from a lateral side or a medial side of an ankle of a patient. (e.g., lateral approach or medial approach). When using this approach a curved osteotomy can be formed in which the osteotomy extends into the bone (e.g., tibia 226 and/or talus 222) at a second angle 1898 (See FIG. 21D) that is perpendicular to a mechanical axis 880 of the bone being resected (e.g., tibia 226 or talus 222). The second angle 1898 may be measured based on a trajectory for how the curved osteotomy extends into the bone within the frontal plane 264. Such a curved osteotomy may be referred to as a straight cut. Alternatively, or in addition, instead of a curved osteotomy, resection guides 1820 may each include resection features for forming angled curved osteotomies.

In one embodiment, the first angle 1896 and the second angle 1898 are determined at least partially based on a bone model of at least a portion of a patient's ankle. Advantageously, a surgeon can define and/or determine the values for the first angle 1896 and/or second angle 1898.

For example, a surgeon may provide these values in a set of user instructions 604. Alternatively, or in addition, the surgeon may review an initial version of an osteotomy system 1800, and based on that review may revise the first angle 1896 and/or second angle 1898. In one embodiment, a preoperative plan can show a surgeon either through an animation and/or a series of images and/or a text description a condition of an ankle and bones of a patient before the osteotomies with one version of an osteotomy system 1800 and a position and/or alignment of the bones and/or ankle after completion of osteotomies using the osteotomy system 1800 and reducing the bones. Accordingly, a surgeon can review the planned positioning and outcome and review the first angle 1896 and/or second angle 1898. Based on the surgeon's professional judgment, the surgeon may decide to revise the first angle 1896 and/or second angle 1898. Alternatively, or in addition, a surgeon may confirm the settings and/or values for the first angle 1896 and/or second angle 1898. For example, after reviewing a preoperative plan with the first angle 1896 and/or second angle 1898 set at a particular set of angles.

In another embodiment, a surgeon may request that two or more resection guides 1820 be fabricated for the osteotomy system 1800 with the intention of using particular resection guides 1820 (i.e., a subset of the two or more resection guides 1820) during the surgical procedure and making the final decision intraoperatively. For example, two or more of the resection guides 1820 may be configured to form angled curved osteotomies using a different first angle 1896.

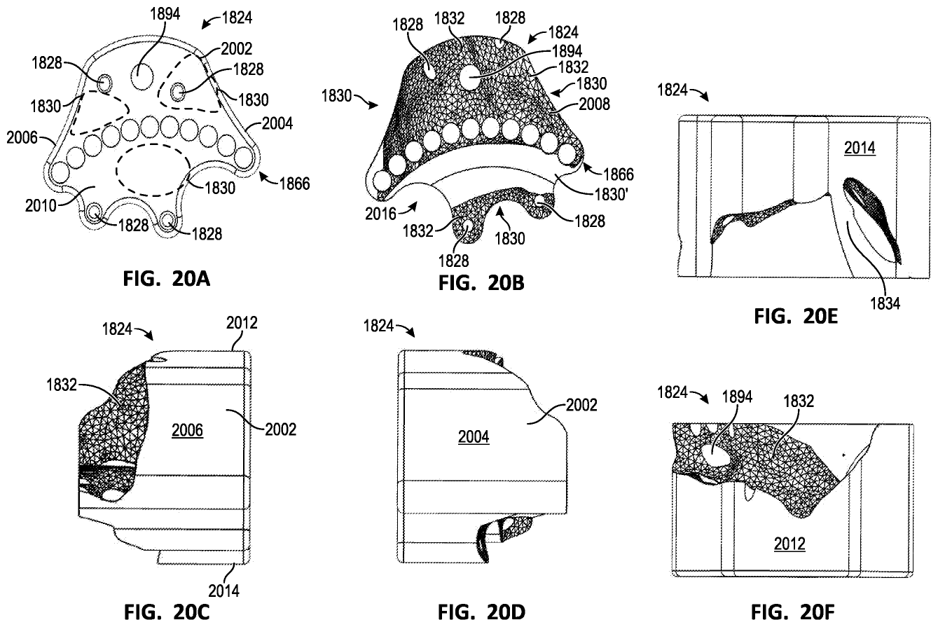

FIGS. 19A-19G illustrate different views of one example first resection guide 1822. In one embodiment, the first resection guide 1822 includes a body 1902 that includes an anterior side 1904, a posterior side 1906, a medial side 1908, a lateral side 1910, a superior side 1912, and an inferior side 1914. Generally, the sides of the first resection guide 1822 refer to the direction the sides face when the first resection guide 1822 is in use. FIG. 19A is an anterior perspective view of one example first resection guide 1822. FIG. 19B is lateral view of the example first resection guide 1822 of FIG. 19A. FIG. 19C is a medial view of the example first resection guide 1822 of FIG. 19A. FIG. 19D is a posterior view of the example first resection guide 1822 of FIG. 19A. FIG. 19E is an anterior view of the example first resection guide 1822 of FIG. 19A. FIG. 19F is a superior view of the example first resection guide 1822 of FIG. 19A. FIG. 19G is an inferior view of the example first resection guide 1822 of FIG. 19A. FIGS. 19C, 19D, 19E, 19F, and 19G include views of the bone engagement surface 1832. In certain embodiments, the first resection guide 1822 can include one or more landmark registration features 1834.

Those of skill in the art will appreciate that one objective in using the resection guides 1820 can be to form a curved osteotomy and/or an angled curved osteotomy. There are a number of ways to configure the resection guides 1820 to reach this objective. In one embodiment, a trajectory for resection of bone can be controlled and/or managed by an angle of the first resection feature 1864 and/or second resection feature 1866 through a body of the resection guide 1820.

Alternatively, whereas in the present disclosure, a resection guide 1820 can be fabricated with a bone facing side (e.g., medial side 1908) that is contoured to a shape, size, contour, and configuration of a surface of the bone, those of skill in the art will appreciate that first resection feature 1864 and/or second resection feature 1866 may extend perpendicular through a body of the resection guide 1820. However, a change in the resection trajectory can be accomplished by changing a distance between a non-bone facing side surface (e.g., lateral side 1910) and a bone facing side (e.g., medial side 1908) surface of a body of the resection guide 1820 such that one end of a resection guide 1820 is closer to the bone than the other side when the resection guide 1820 is deployed. Thus, by varying the depth of the body across the bone facing side (e.g., medial side 1908) of the body, a designer can cause the a resection feature to contact the bone at a desired angle (e.g., first angle 1896 and/or second angle 1898).

FIGS. 19A-19C illustrate that the example first resection guide 1822 includes one or more stabilizers 1892 and one or more sights 1894. In the example first resection guide 1822 the bone attachment features 1828 are configured such that holes/openings in the body 1902 are parallel to each other and therefore the pins deployed into these holes are also parallel to each other. Having parallel pins can be advantageous because the parallel pins can enable the first resection guide 1822 to be readily removed by sliding the body 1902 off of the pins. In this manner, the pins can remain in the bone(s) and can serve as reference features. However, the parallel pins can also result in the body 1902 being unstable and moving too much during the osteotomy. For example, the body 1902 may move away from the bone(s) along the pins (a translation referred to as riding up).

Accordingly, to retain the advantages of parallel pins for the bone attachment features 1828 and provide a stable first resection guide 1822, the first resection guide 1822 can include one or more stabilizers 1892. In one embodiment, a stabilizer 1892 is an opening in the body 1902 configured to accept a pin deployed through the opening and into the bone(s). Advantageously, the opening extends through the body 1902 at an oblique angle relative to the angle that openings for one or more bone attachment features 1828 pass through the body. This angle pin of the stabilizer 1892 ensures that the body 1902 remains stable against the bone(s) for the osteotomy. Of course, the first resection guide 1822 can include one or more stabilizers 1892.

The sights 1894 serve to assist a surgeon in reviewing, confirming, and/or validating a position of the first resection guide 1822 relative to other anatomical structures of the patient (e.g., bones). In one embodiment, sights 1894 are used during a surgical procedure and can be used together with medical imaging to check position, alignment, trajectory, or the like. In one embodiment, a surgeon may provisionally position the first resection guide 1822 and then use flouroscopy to visually check or confirm a position of the first resection guide 1822 relative to another bone or other bones of the patient. In certain embodiments, the flouroscopy machine (e.g., C-arm) can include reference features that key off of certain markers or anatomical features of the patient to ensure that the flouroscopy image is the view desired for the surgeon. A surgeon may use the flouroscopy machine and locate a sight 1894 to determine whether or not the first resection guide 1822 is in a desired position. In one embodiment, the flouroscopy machine may include a scope or view finder or corresponding sight (e.g., cross hairs) that a surgeon can align with the sight 1894 to do a visual check and/or confirmation of the position of the first resection guide 1822. After this visual check, a surgeon may secure the first resection guide 1822 to one or more bones of the patient (i.e., deploy pins in bone attachment features 1828).

In the illustrated embodiment, the sights 1894 is implemented as an opening having a predefined diameter, an opening that is positioned in a predetermined location of the body 1902 and that extends from a lateral side 1910 to a medial side 1908 of the body 1902. In the illustrated embodiment, the sight 1894 also aligns with the alignment guide 1874 and/or a shaft 1878 deployed in the alignment guide 1874. In one embodiment, a sight 1894 can include cross-hairs positioned in the center of the sight 1894.

FIG. 19B illustrates that the first resection guide 1822 can include one or more bone engagement features 1830. These bone engagement features 1830 can each include a bone engagement surface 1832. As described herein, the number, size, and/or configuration of the bone engagement features 1830 can be determined based, at least in part, on a user instructions 604.

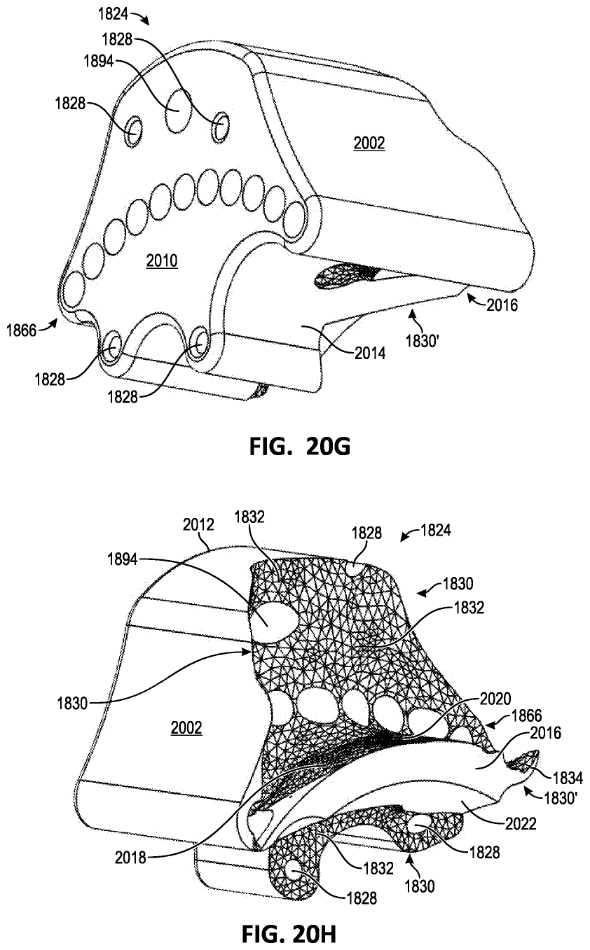

FIGS. 20A-20G illustrate different views of one example second resection guide 1824. In one embodiment, the second resection guide 1824 includes a body 2002 that includes an anterior side 2004, a posterior side 2006, a medial side 2008, a lateral side 2010, a superior side 2012, and an inferior side 2014. Generally, the sides of the second resection guide 1824 refer to the direction the sides face when the second resection guide 1824 is in use. FIG. 20A is lateral view of the example second resection guide 1824 of FIG. 20A. FIG. 20B is a medial view of the example second resection guide 1824 of FIG. 20A. FIG. 20C is a posterior view of the example second resection guide 1824 of FIG. 20A. FIG. 20D is an anterior view of the example second resection guide 1824 of FIG. 20A. FIG. 20E is a superior view of the example second resection guide 1824 of FIG. 20A. FIG. 20F is an inferior view of the example second resection guide 1824 of FIG. 20A. FIG. 20G is an anterior lateral perspective view of the example second resection guide 1824. FIG. 20H is an anterior medial perspective view of the example second resection guide 1824.

FIGS. 20B-20F and 20H include views of the bone engagement surface 1832. In certain embodiments, the second resection guide 1824 can include one or more landmark registration features 1834.

Those of skill in the art will appreciate that one objective in using the resection guides 1820 can be to form a curved osteotomy and/or an angled curved osteotomy. There are a number of ways to configure the resection guides 1820 to reach this objective. In one embodiment, a trajectory for resection of bone can be controlled and/or managed by an angle of the first resection feature 1864 and/or second resection feature 1866 through a body of the resection guide 1820.

Alternatively, whereas in the present disclosure, a resection guide 1820 can be fabricated with a bone facing side (e.g., medial side 2008) that is contoured to a shape, size, contour, and configuration of a surface of the bone, those of skill in the art will appreciate that first resection feature 1864 and/or second resection feature 1866 may extend perpendicular through a body of the resection guide 1820. However, a change in the resection trajectory can be accomplished by changing a distance between a non-bone facing side (e.g., lateral side 2010) surface and a bone facing side (e.g., medial side 2008) surface of a body of the resection guide 1820 such that one end of a resection guide 1820 is closer to the bone than the other side when the resection guide 1820 is deployed. Thus, by varying the depth of the body across the bone facing side (e.g., medial side 2008) of the body, a designer can cause the a resection feature to contact the bone at a desired angle (e.g., first angle 1896 and/or second angle 1898).

One example of this variation in a depth of the body can be seen in the example resection guide 1820 (i.e., second resection guide 1824) in FIG. 20C. FIG. 20C illustrates a left side view (posterior side 2006, because the second resection guide 1824 is configured for use on a lateral side of a patient's ankle) of the second resection guide 1824. Note that the body 2002 is shorter near the superior side 2012 than near the inferior side 2014. In other words, the posterior side 2006 is wider at or near the inferior end than at or near the superior end. This difference in width can be used to set a trajectory for the second resection feature 1866 relative to one or more bones.

FIGS. 20A-20C illustrate that the example second resection guide 1824 includes one or more sights 1894. The second resection guide 1824 may not need stabilizers 1892 because pins for the bone attachment features 1828 may be deployed at oblique angles relative to each other.

The sights 1894 serve to assist a surgeon in reviewing, confirming, and/or validating a position of the second resection guide 1824 relative to other anatomical structures of the patient (e.g., bones). In one embodiment, sights 1894 are used during a surgical procedure and can be used together with medical imaging to check position, alignment, trajectory, or the like. In one embodiment, a surgeon may provisionally position the second resection guide 1824 and then use flouroscopy to visually check or confirm a position of the second resection guide 1824 relative to another bone or other bones of the patient. In certain embodiments, the flouroscopy machine (e.g., C-arm) can include reference features that key off of certain markers or anatomical features of the patient to ensure that the flouroscopy image is the view desired for the surgeon. A surgeon may use the flouroscopy machine and locate a sight 1894 to determine whether or not the second resection guide 1824 is in a desired position. In one embodiment, the flouroscopy machine may include a scope or view finder or corresponding sight (e.g., cross hairs) that a surgeon can align with the sight 1894 to do a visual check and/or confirmation of the position of the second resection guide 1824. After this visual check, a surgeon may secure the second resection guide 1824 to one or more bones of the patient (i.e., deploy pins in bone attachment features 1828).

In the illustrated embodiment, the sights 1894 is implemented as an opening having a predefined diameter, an opening that is positioned in a predetermined location of the body 2002 and that extends from a lateral side 2010 to a medial side 2008 of the body 2002. In one embodiment, a sight 1894 can include cross-hairs positioned in the center of the sight 1894.

FIGS. 20A, 20B illustrate that the second resection guide 1824 can include one or more bone engagement features 1830. These bone engagement features 1830 can each include a bone engagement surface 1832. As described herein, the number, size, and/or configuration of the bone engagement features 1830 can be determined based, at least in part, on a user instructions 604.

FIGS. 20B, 20G, and 20H illustrate an example embodiment of the second resection guide 1824 illustrated in FIG. 20. The second resection guide 1824 includes a particular type of bone engagement feature 1830′. In the illustrated embodiment, the bone engagement feature 1830′ includes an insert 2016 and a bone engagement surface 2018.

The insert 2016 is a structure that is configured to extend into a space such as a curved osteotomy. In another embodiment, an insert 2016 is configured to extend into a space between bones of a joint. Advantageously, the insert 2016 assists a surgeon in confirming that the second resection guide 1824 is in a desired position, prior to securing the second resection guide 1824 to one or more bones and/or performing an osteotomy.

In the illustrated embodiment, the second resection guide 1824 is configured to be used for a second osteotomy after a first osteotomy is performed (e.g., using the first resection guide 1822). The second resection guide 1824 uses resected bone surfaces from the first osteotomy to position and/or orient the second resection guide 1824. Consequently, the bone engagement surface 2018 of the bone engagement feature 1830′ includes at least one surface configured to engage with one or more surfaces of a first osteotomy. In one embodiment, the bone engagement surface 2018 is configured to engage with both sides of a curved osteotomy. As discussed in more detail herein, a surgeon may use a first resection guide 1822 to form a first osteotomy, a curved osteotomy, that cuts into an end of one bone (e.g., a talus 222), an end of two bones (e.g., a talus 222 and a tibia 226), into a portion of a single bone (e.g., a talus 222 or tibia 226).

Where the curved osteotomy resects ends of two bones of a joint, the curved osteotomy has a superior surface/side (resected surface of one bone) and an inferior surface/side (resected surface of another bone). In certain embodiments, the bone engagement surface 2018 is configured to engage with both sides of the curved osteotomy. As shown in the example of FIG. 21D, these side surfaces of the osteotomy can be generally smooth and curved or rounded. Accordingly, FIG. 20H illustrates that the bone engagement surface 2018, in one embodiment, includes a superior bone engagement surface 2020 and an inferior bone engagement surface 2022.

In certain embodiments, a resection guide 1820 (e.g., first resection guide 1822 and/or second resection guide 1824) can include a plurality of bone engagement surfaces that are either independent or part of a bone engagement feature 1830. In one embodiment, such as the example of FIGS. 20B and 20H, the resection guide 1820 can include a first bone engagement surface (e.g., superior bone engagement surface 2020) on one side and a second bone engagement surface (e.g., inferior bone engagement surface 2022) on another side. The two sides that include bone engagement surfaces may be opposite sides as with the superior bone engagement surface 2020 and the inferior bone engagement surface 2022. Alternatively, or in addition, the two sides that include bone engagement surfaces may be adjacent sides such as superior bone engagement surface 2020 on a bone engagement feature 1830′ of an insert 2016 and a bone engagement surface 1832 on a medial side 2008 of a body 2002.

Advantageously, the bone engagement feature 1830′ with its insert 2016 can be modeled, designed, and/or fabricated based on at least a portion of a bone model of one or more bones of a patient. In particular, the bone engagement feature 1830′ can be modeled in relation to bone(s) of a patient after formation of a first osteotomy (e.g., a curved osteotomy). Thus, new space or openings formed by the first osteotomy can be used as reference features to position and/or register the second resection guide 1824 using the bone engagement feature 1830′.

FIGS. 21A-21E illustrate different views of a surgical osteotomy procedure using the osteotomy system of FIG. 18, according to one embodiment.

FIG. 21A illustrates a stage of an osteotomy surgical procedure in which a first resection guide 1822 is positioned on a lateral surface of an ankle joint. The first resection guide 1822 is positioned for an osteotomy of a talus 222. However, the osteotomy of the talus 222 may also resect a portion of a distal end of a tibia 226.

In the illustrated embodiment, the surgeon has made an incision that permits access to a lateral surface of a distal end of a tibia and lateral surface of a proximal end of a talus of a patient's foot. In addition, a surgeon has formed an osteotomy in the fibula 228 and retracted a bone fragment of the fibula 228 to gain access to the ankle.

Pins of four bone attachment features 828 (e.g., one or more tibial bone attachment features and one or more talus bone attachment features) have also been deployed to secure the first resection guide 1822 to the tibia 226 and the talus 222. Note that the first resection guide 1822 includes an alignment guide 1874 with a shaft 1878 inserted. A surgeon can now visually check manually, and/or with flouroscopy, a position of the first resection guide 1822 in relation to a mechanical axis 880 of the tibia 226. A surgeon can now form a curved osteotomy in a sagittal plane 262 using the first resection feature 1864 and a cutting tool such as a burr.

FIG. 21B illustrates the ankle joint after formation of a curved osteotomy and removal of the first resection guide 1822. In one embodiment, certain pins for bone attachment features 1828 such as those for the tibia 226 may remain in the bone. FIG. 21B illustrates that the curved osteotomy 2100 has been formed on or near a superior surface of the talus 222. In certain embodiments, this curved osteotomy 2100 can be used for or by other instruments during the surgical procedure.

In one embodiment, FIG. 21C illustrates a stage in which a second resection guide 1824 is positioned on a lateral side of the ankle. The second resection guide 1824 can be secured using one or more common pins that were used for the first resection guide 1822 and/or new pins may be deployed to secure the second resection guide 1824. In one embodiment, pins for bone attachment features 1828 in the tibia may be deployed using the bone attachment features 1828 at an angle that ensures that the second resection guide 1824 is securely coupled to the bone(s). In the illustrated embodiment, the second resection guide 1824 is positioned to resect a portion of an end of the tibia 226 in preparation for an ankle fusion. Next, a surgeon may form a second osteotomy, such as an angled curved osteotomy 2102. The angled curved osteotomy 2102 formed in the bone(s) may not be perpendicular to a mechanical axis 880 of the tibia 226.

In one embodiment, FIG. 21D illustrates a stage in which a first resection guide 1822 and a second resection guide 1824 have been removed. A curved osteotomy (e.g., first osteotomy) has been formed at the proximal end of the talus 222 and an angled curved osteotomy (e.g., second osteotomy) has been formed at the distal end of the tibia 226.

In one embodiment, the angled curved osteotomy is formed by configuring the second resection feature 1866 such that the opening(s) (e.g., curved slot and/or plurality of openings) that extend through the body 2002 do so at a first angle 1896. The first angle 1896 can be configured based on a bone model of one or more bones of the patient. The first angle 1896 of the opening(s) in the second resection feature 1866 guide a cutting tool to form an angled curved osteotomy in the tibia 226.

Alternatively, or in addition, a curved osteotomy 2100 can be formed by configuring the first resection feature 1864 such that the opening(s) (e.g., curved slot and/or plurality of openings) that extend through the body 1902 do so at a second angle 1898. The second angle 1898 can be configured based on a bone model of one or more bones of the patient. The second angle 1898 of the opening(s) in the first resection feature 1864 guide a cutting tool to form a curved osteotomy in the talus 222.

FIG. 21D illustrates one example of how the first resection feature 1864 and/or second resection feature 1866 can be configured to form a curved osteotomy and an angled curved osteotomy. In the illustrated embodiment, the first resection guide 1822 includes a first resection feature 1864 that guides a cutting tool to resect at least part of a talus 222 at a second angle 1898. Note that the second angle 1898 is perpendicular to the mechanical axis 880 (and parallel to a medial-lateral axis ML). The first resection feature 1864 enables a surgeon to form a curved osteotomy that can be referred to as a “straight cut”. The curved osteotomy curves within the sagittal plane 262 and extends into the bone parallel with the ML axis.

In the illustrated embodiment, the second resection guide 1824 includes a second resection feature 1866 that guides a cutting tool to resect at least part of a tibia 226 at a first angle 1896. Note that the first angle 1896 is not perpendicular (i.e., is oblique) to the mechanical axis 880 (and to a medial-lateral axis ML). The second resection feature 1866 enables a surgeon to form an angled curved osteotomy that can be referred to as an “angled cut”. The curved osteotomy curves within the sagittal plane 262 and extends into the bone at an angle in relation to the ML axis.

Said another way, the first resection feature 1864 extends perpendicularly from the medial side 1908 of the first resection guide 1822 to the lateral side 1910 of the first resection guide 1822 at the second angle 1898 and the second resection feature 1866 extends from the medial side 2008 of the second resection guide 1824 to the lateral side 2010 of the second resection guide 1824 at the first angle 1896.

In one embodiment, FIG. 21E illustrates a stage in which a first resection guide 1822 and a second resection guide 1824 have been removed. A curved osteotomy (e.g., first osteotomy) has been formed at the proximal end of the talus 222 and an angled curved osteotomy (e.g., second osteotomy) has been formed at the distal end of the tibia 226. The curved osteotomy and the angled curved osteotomy are reduced at the resection interface 884. Advantageously, a minimal amount of bone of the talus 222 and/or the tibia 226 has been removed and a stable, complete reduction is achieved. Note reference features, fixation pins, permanent and/or temporary fixation is omitted from the view in FIG. 21E but may be included in certain embodiments and/or may be added to hold the reduction place for healing and fusion.

FIG. 21E illustrates that the first angle 1896 and second angle 1898 have been selected, determined, defined (e.g., using bone models of the patient) such that reduction of the curved osteotomy and the angled curved osteotomy remediates a deformity of the patient. Note that in comparing FIGS. 21D to 21E, the alignment and/or position of the calcaneus 224 has been changed and the calcaneus 224 has been translated more in line with the mechanical axis 880 of the tibia 226. A first dome osteotomy (i.e., curved osteotomy) and a second dome osteotomy (i.e., angled curved osteotomy) have been brought together to provide a deformity correction. Those of skill in the art will appreciate that forming the curved osteotomy and/or the angled curved osteotomy without the aid of the first resection guide 1822 and/or second resection guide 1824 may have required resection of the bone(s) by manual free-hand. Advantageously, the reduction and subsequent fusion using the first resection guide 1822 and/or second resection guide 1824 can result in a more satisfactory fusion of an ankle joint.

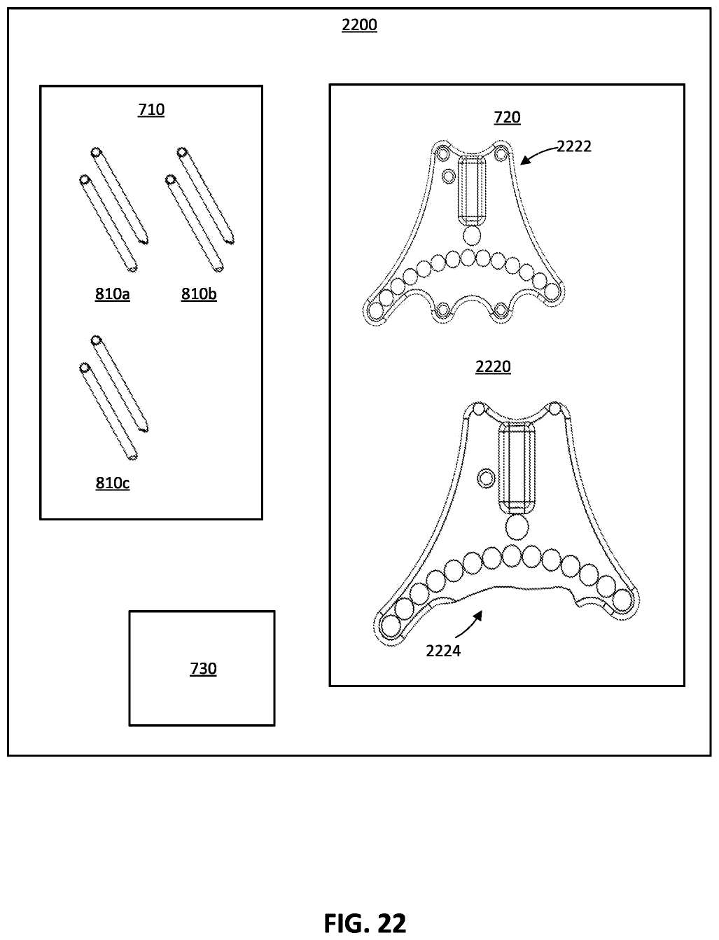

FIG. 22 illustrates an exemplary system for an osteotomy 2200 using a medial approach, according to one embodiment. In one embodiment, the exemplary ankle fusion osteotomy system 2200 is specifically designed for use on a medial side of the patient's ankle (e.g., using a medial approach). Those of skill in the art will appreciate that the same or a similar exemplary ankle fusion osteotomy system 2200 can be used on a lateral side of the patient's ankle (e.g., using a lateral approach). Those of skill in the art will also appreciate that a surgical procedure that conducts an ankle fusion on an ankle from a medial or lateral approach generally includes steps to perform an osteotomy on the malleolus (medial or lateral depending on the approach) and retracting or holding a distal end of the malleolus out of the way for the osteotomy steps on the tibia 226 and/or talus 222.

The osteotomy system 2200 can include one or more fasteners 810 and/or two or more sets of fasteners (810a, 810b, 810c), one or more resection guides 2220, and zero or one or more complementary components 730. In the illustrated embodiment, the resection guides 2220 include a first resection guide 2222 and a second resection guide 2224. The resection guides 2220 may also include one or more of resection features 2226, bone attachment features 2228, bone engagement feature 2230, and/or landmark registration feature 2234. In certain embodiments, a bone engagement feature 2230 can include a bone engagement surface 2232.

While specific embodiments of complementary components 730 are not specifically shown here in relation to the osteotomy system 2200, those of skill in the art will appreciate that complementary components 730 can be similar in feature, design, implementation, configuration, and purpose as those described in relation to the osteotomy system 700 and can be used for the osteotomy system 2200. Thus, the osteotomy system 2200 can include one or more alignment guides 740, rotation guides 750, correction guides 760, compression guides 770, positioning guides 780, fixation guides 790, navigation guides 792, implants 794, or the like.

Either or both of the resection guides 2220 may be custom patient-specific resection guides made for a particular patient and/or for a particular surgical procedure. Various aspects of the resection guides 2220 may be patient-specific, including, but not limited to, an angle and/or orientation for a resection feature of the resection guide 2220, a position of the resection feature, a depth of the resection feature, a size of the resection guide 2220, a configuration and/or composition of a bone contacting surface such as a bone engagement surface of the resection guide 2220, and the like.

Either or both of the resection guides 2220 of osteotomy system 2200 can include some or all of the same or substantially the same features, aspects, alternatives, and/or components as other example resection guides described herein with like components including the same or similar reference numerals. Accordingly, the resection guide 2222 can or may include a first resection feature 2264, a second resection feature 2266, one or more bone attachment features 2228, one or more bone engagement features 2230, one or more alignment guides 2274, one or more shafts 2278, one or more openings 2276, one or more resection guards 2270 (not shown), one or more fastener guides 2290 (not shown), and the like. The bone engagement features 2230 may include one or more bone engagement surfaces 2232. Alternatively, or in addition, either or both of the resection guides 2220 can include one or more stabilizers 2292 and one or more sights 2294.

The resection guides 2222 may each include a body that includes an anterior side, a posterior side, a medial side, a lateral side, a superior side, and an inferior side. In certain embodiments, a first resection guide 2222 may be combined with a second resection guide 2224 into a single resection guide 2220.

The resection guide 2222 and/or resection guide 2224 of osteotomy system 2200 can include some, or all of the same or substantially the same features, aspects, and/or components as the resection guides 820 and/or first resection guide 1822 and/or second resection guide 1824 described herein with like components including the same reference numerals. Accordingly, the resection guides 2220 can or may include one or more resection features, one or more bone attachment features, one or more bone engagement features, one or more alignment guides, one or more shafts, one or more openings, one or more resection guards, one or more fastener guides, and/or the like.

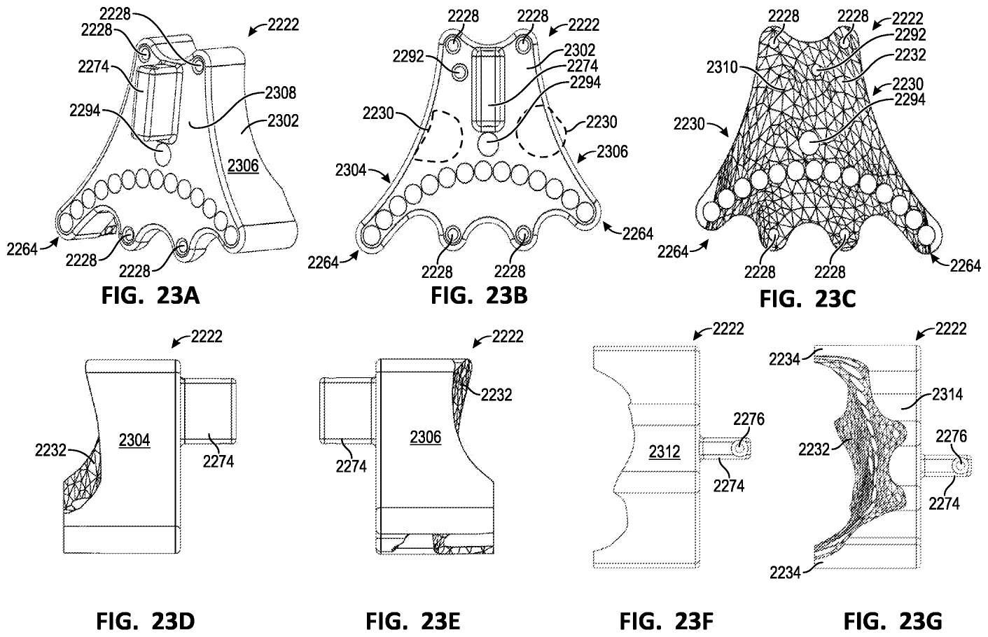

FIGS. 23A-23G illustrate different views of one example first resection guide 2222. In one embodiment, the first resection guide 2222 includes a body 2302 that includes an anterior side 2304, a posterior side 2306, a medial side 2308, a lateral side 2310, a superior side 2312, and an inferior side 2314. Generally, the sides of the first resection guide 2222 refer to the direction the sides face when the first resection guide 2222 is in use. FIG. 23A is an anterior perspective view of one example first resection guide 2222. FIG. 23B is a medial view of the example first resection guide 2222 of FIG. 23A. FIG. 23C is a lateral view of the example first resection guide 2222 of FIG. 23A. FIG. 23D is an anterior view of the example first resection guide 2222 of FIG. 23A. FIG. 23E is a posterior view of the example first resection guide 2222 of FIG. 23A. FIG. 23F is a superior view of the example first resection guide 2222 of FIG. 23A. FIG. 23G is an inferior view of the example first resection guide 2222 of FIG. 23A. FIGS. 23C, 23D, 23E, and 23G include views of the bone engagement surface 2232. In certain embodiments, the first resection guide 2222 can include one or more landmark registration features 2234.

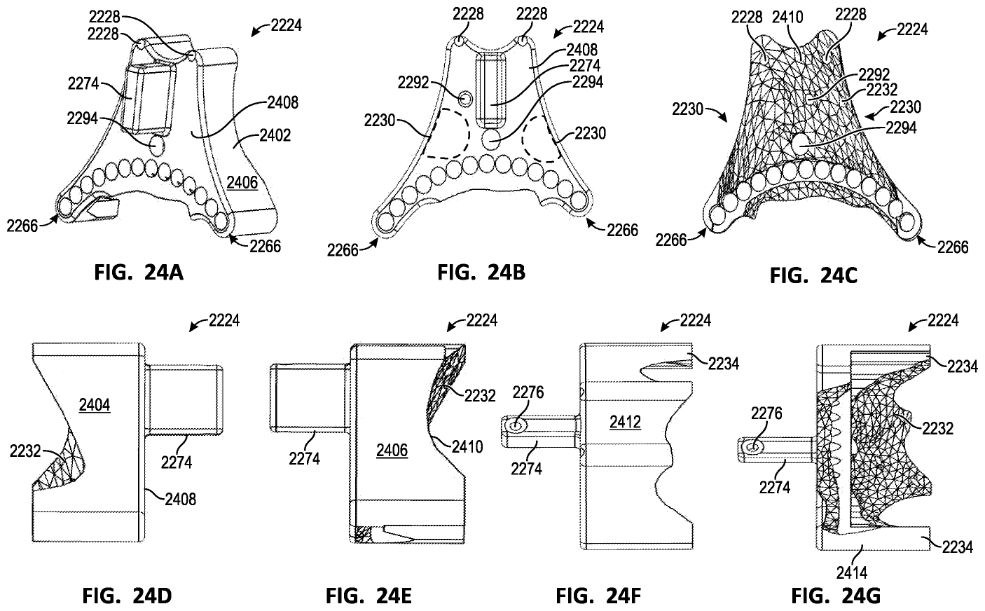

FIGS. 24A-24G illustrate different views of one example second resection guide 2224. In one embodiment, the second resection guide 2224 includes a body 2402 that includes an anterior side 2404, a posterior side 2406, a medial side 2408, a lateral side 2410, a superior side 2412, and an inferior side 2414. Generally, the sides of the second resection guide 2224 refer to the direction the sides face when the second resection guide 2224 is in use. FIG. 24A is an anterior perspective view of one example second resection guide 2224. FIG. 24B is a medial view of the example second resection guide 2224 of FIG. 24A. FIG. 24C is a lateral view of the example second resection guide 2224 of FIG. 24A. FIG. 24D is an anterior view of the example second resection guide 2224 of FIG. 24A. FIG. 24E is a posterior view of the example second resection guide 2224 of FIG. 24A. FIG. 24F is a superior view of the example second resection guide 2224 of FIG. 24A. FIG. 24G is an inferior view of the example second resection guide 2224 of FIG. 24A. FIGS. 24C, 24D, 24E, and 24G include views of the bone engagement surface 2232. In certain embodiments, the second resection guide 2224 can include one or more landmark registration features 2234.

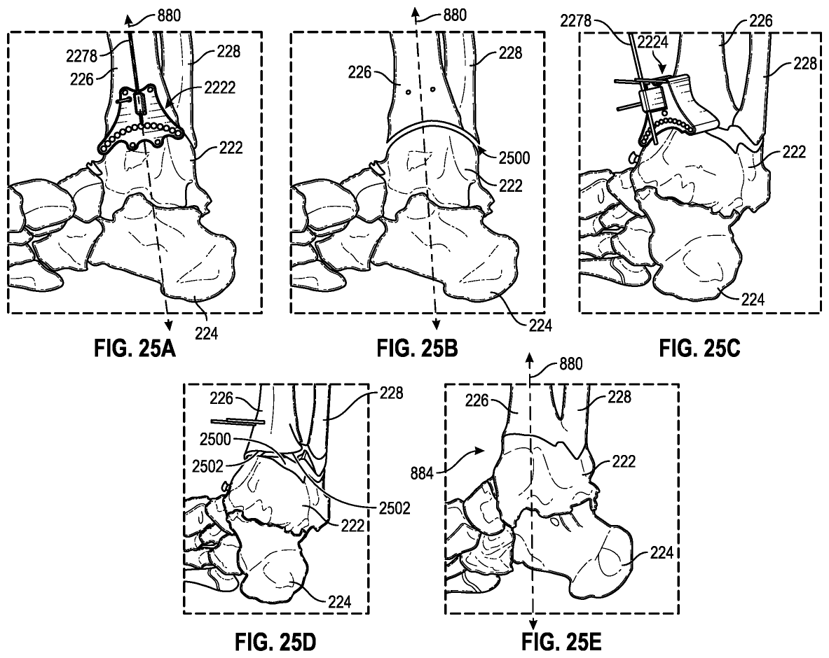

FIGS. 25A-25E illustrate different views of a surgical osteotomy procedure using the osteotomy system of FIG. 22, according to one embodiment.

FIG. 25A illustrates a stage of an osteotomy surgical procedure in which a first resection guide 2222 is positioned on a medial surface of an ankle joint. The first resection guide 2222 is positioned for an osteotomy of a talus 222 or a tibia 226. However, the osteotomy of the talus 222 may also resect a portion of a distal end of a tibia 226, and vice versa.

In the illustrated embodiment, the surgeon has made an incision that permits access to a lateral surface of a distal end of a tibia and lateral surface of a proximal end of a talus of a patient's foot. In one embodiment, the surgical procedure may be a revision procedure of a prior ankle fusion. Accordingly, one of the first resection guide 2222 and/or the second resection guide 2224 can form an osteotomy on one or the other of a tibia 226 and a talus 222.

Pins of four bone attachment features 828 (e.g., one or more tibial bone attachment features and one or more talus bone attachment features) have also been deployed to secure the first resection guide 2222 to bone. Note that the first resection guide 2222 includes an alignment guide 2274 with a shaft 2278 inserted. A surgeon can now visually check manually, and/or with flouroscopy, a position of the first resection guide 2222 in relation to a mechanical axis 880 of the tibia 226. A surgeon can now form a curved osteotomy in a sagittal plane 262 using the first resection feature 2264 and a cutting tool such as a burr.

FIG. 25B illustrates the ankle joint after formation of a curved osteotomy and removal of the first resection guide 2222. In one embodiment, certain pins for bone attachment features 1828 such as those for the tibia 226 may remain in the bone. FIG. 25B illustrates that the curved osteotomy 2500 has been formed on or near a superior surface of the talus 222. Alternatively, the curved osteotomy 2500 can be formed along a length of tibia 226 and/or talus 222. In certain embodiments, this curved osteotomy 2100 can be used for, or by, other instruments during the surgical procedure.

In one embodiment, FIG. 25C illustrates a stage in which a second resection guide 2224 is positioned on a lateral side of the ankle. The second resection guide 2224 can be secured using one or more common pins that were used for the first resection guide 2222 and/or new pins may be deployed to secure the second resection guide 2224. In one embodiment, pins for bone attachment features 2228 in the tibia may be deployed using the bone attachment features 2228 at an angle that ensures that the second resection guide 2224 is securely coupled to the bone(s). In the illustrated embodiment, the second resection guide 2224 is positioned to resect a portion of an end of the tibia 226 in preparation for an ankle fusion. Next, a surgeon may form a second osteotomy, such as an angled curved osteotomy 2502. The angled curved osteotomy 2502 formed in the bone(s) may not be perpendicular to a mechanical axis 880 of the tibia 226.

In one embodiment, FIG. 25D illustrates a stage in which a first resection guide 2222 and a second resection guide 2224 have been removed. A curved osteotomy 2500 (e.g., first osteotomy) has been formed at or near a proximal end of the talus 222 and an angled curved osteotomy 2502 (e.g., second osteotomy) has been formed at or near a distal end of the tibia 226.

In one embodiment, the angled curved osteotomy 2502 is formed by configuring the second resection feature 2266 such that the opening(s) (e.g., curved slot and/or plurality of openings) that extend through the body 2402 do so at a first angle. The first angle can be configured based on a bone model of one or more bones of the patient. The first angle of the opening(s) in the second resection feature 2266 guide a cutting tool to form an angled curved osteotomy 2502.

Alternatively, or in addition, a curved osteotomy 2500 can be formed by configuring the first resection feature 2264 such that the opening(s) (e.g., curved slot and/or plurality of openings) that extend through the body 2302 do so at a second angle. The second angle can be configured based on a bone model of one or more bones of the patient. The second angle of the opening(s) in the first resection feature 2264 guide a cutting tool to form a curved osteotomy 2500.

FIG. 25D illustrates one example of how the first resection feature 2264 and/or second resection feature 2266 can be configured to form a curved osteotomy and an angled curved osteotomy. In the illustrated embodiment, the first resection guide 2222 includes a first resection feature 2264 that guides a cutting tool to resect at least part of a talus 222 at a second angle. Note that the second angle may be perpendicular to the mechanical axis 880 (and parallel to a medial-lateral axis ML). The first resection feature 2264 enables a surgeon to form a curved osteotomy that can be referred to as a “straight cut”. The curved osteotomy curves within the sagittal plane 262 and extends into the bone parallel with the ML axis.

In the illustrated embodiment, the second resection guide 2224 includes a second resection feature 2266 that guides a cutting tool to resect at least part of a tibia 226 at a first angle. Note that the first angle is not perpendicular (i.e., the first angle is oblique) to the mechanical axis 880 (and to a medial-lateral axis ML). The second resection feature 2266 enables a surgeon to form an angled curved osteotomy that can be referred to as an “angled cut”. The curved osteotomy curves within the sagittal plane 262 and extends into the bone at an angle in relation to the ML axis.

In one embodiment, FIG. 25E illustrates a stage in which a first resection guide 2222 and a second resection guide 2224 have been removed. A curved osteotomy (e.g., first osteotomy) has been formed at or near a proximal end of the talus 222 and an angled curved osteotomy (e.g., second osteotomy) has been formed at or near a distal end of the tibia 226. The curved osteotomy and the angled curved osteotomy are reduced at the resection interface 884. Advantageously, a minimal amount of bone of the talus 222 and/or the tibia 226 has been removed and a stable, complete reduction is achieved. Note reference features, fixation pins, permanent and/or temporary fixation is omitted from the view in FIG. 25E but may be included in certain embodiments and/or may be added to hold the reduction place for healing and fusion.

FIG. 25E illustrates that a first angle and second angle can be selected, determined, and defined (e.g., using bone models of the patient) such that reduction of the curved osteotomy and the angled curved osteotomy remediates a deformity of the patient. Note that in comparing FIGS. 25D to 25E, the alignment and/or position of the calcaneus 224 has been changed and the calcaneus 224 has been translated more in line with the mechanical axis 880 of the tibia 226. A first dome osteotomy (i.e., curved osteotomy) and a second dome osteotomy (i.e., angled curved osteotomy) have been brought together to provide a deformity correction. Those of skill in the art will appreciate that forming the curved osteotomy and/or the angled curved osteotomy without the aid of the first resection guide 2222 and/or second resection guide 2224 may have required resection of the bone(s) by manual free-hand. Advantageously, the reduction and subsequent fusion using the first resection guide 2222 and/or second resection guide 2224 can result in a more satisfactory fusion of an ankle joint.

Those of skill in the art will appreciate that embodiments of the system disclosed herein can be used on humans and animals and on bones that are relatively small in comparison to other bones of the body (e.g., bones of the foot and hand). Advantageously, the embodiments of the system seek to minimize the number of fasteners or pins placed within the bones of a patient by planning a surgical procedure such that pins or fasteners placed in one stage are and/or can be reused in subsequent stages. Consequently, pins initially deployed can remain in the bone or bone fragment as instruments are deployed and/or subsequent stages of the surgical procedure are performed.

Advantageously, because the present disclosure uses a bone model of the patient's bones the sizes, dimensions, lengths and configurations of the components of the example systems can each be changed, adapted, revised, and/or customized to meet the needs and/or preferences of the patient and/or surgeon. Advantageously, using the apparatus, systems, and/or methods of the present disclosure the surgeon may have a preoperative plan that identifies which specific bone screw (length, width, diameter, thread, pitch, etc.) to use for the fasteners.

Advantageously, the present disclosure provides an apparatus, system, and/or method that can remediate a condition in a patient's foot. Those of skill in the art will appreciate that the methods, processes, apparatuses, systems, devices, and/or instruments of the present disclosure can be used to address a variety of conditions in a variety of procedures and/or parts of the body of the patient.

Conventionally, correction methods, systems, and/or instrumentation for a condition such as, for example, a bunion and/or a hallux valgus, face several challenges. One example is how to cut the bone such that the cut faces have a desired angle in relation to each other. Advantageously, the present disclosure can address many, if not all, of these challenges to assist a surgeon in performing the surgical procedure and improve the quality of patient care and outcomes.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. An apparatus for an ankle fusion procedure to remediate a bone condition present in a patient's foot, comprising:
a resection guide comprising:
a body having an anterior side, a posterior side, a medial side, a lateral side, a superior side, and an inferior side;
a first resection feature configured to guide a cutting tool to form a first osteotomy in a first bone, the first osteotomy defined based at least partially on user directions and at least partially on a bone model of at least a portion of the first bone, the bone model based on medical imaging of a patient's foot; and
a first bone attachment feature configured to secure the resection guide to the first bone.

2. The apparatus of claim 1, further comprising:
a bone engagement feature configured to engage with at least a portion of the first bone at a position that substantially matches a model position of a model of the resection guide engaging the bone model.

3. The apparatus of claim 2, wherein the bone engagement feature comprises:
a bone engagement surface configured to at least partially match a contour of a surface of the first bone when the resection guide is positioned for use; and
a body section extending from the body to support the bone engagement surface.

4. The apparatus of claim 2, wherein the bone engagement feature comprises a bone probe configured to at least partially engage with a landmark associated with the first bone.

5. The apparatus of claim 2, wherein the bone engagement feature is configured based at least partially on the user directions and at least partially on the bone model.

6. The apparatus of claim 1, further comprising a plurality of bone engagement features, at least one of the plurality of bone engagement features configured based on the user directions.

7. The apparatus of claim 1, further comprising:
a second resection feature coupled to the body and configured to guide the cutting tool to form a second osteotomy in a second bone, the second osteotomy shaped to form a resection interface with the first osteotomy;
a second bone attachment feature configured to secure the resection guide to the second bone; and
wherein the second bone is part of a joint that includes the first bone.

8. A system for an ankle fusion procedure to remediate a bone condition present in a patient's foot, comprising:
a tibial resection guide comprising:
a body having an anterior side, a posterior side, a medial side, a lateral side, a superior side, and an inferior side;
a tibia resection feature configured to guide a cutting tool to form a first osteotomy in a tibia, the tibia resection feature extending through the tibial resection guide from the anterior side to the posterior side at a position at least partially determined based on a bone model of at least a portion of the patient's foot, the bone model based on medical imaging of the patient's foot;
a tibial bone attachment feature configured to secure the tibial resection guide to the tibia;
a talus resection guide comprising:
a body having an anterior side, a posterior side, a medial side, a lateral side, a superior side, and an inferior side;
a talus resection feature configured to guide the cutting tool to form a second osteotomy in a talus, the talus resection feature extending through the talus resection guide from the anterior side to the posterior side at a position at least partially determined based on the bone model, the second osteotomy configured to cooperate with the first osteotomy to form a resection interface between the tibia and the talus; and
a talus bone attachment feature configured to secure the talus resection guide the talus.

9. The system of claim 8, wherein the resection interface consists of a resected distal end of the tibia and a resected proximal end of the talus.

10. The system of claim 8, wherein the resection interface comprises a polygonal cross-section taken along an anterior-posterior axis that extends from the anterior side to the posterior side of the body.

11. The system of claim 8, wherein the resection interface comprises a curve shape cross-section taken along an anterior-posterior axis that extends from the anterior side to the posterior side of the body.

12. The system of claim 8, further comprising an alignment guide coupled to one of the tibial resection guide and the talus resection guide, the alignment guide configured to indicate an orientation of one of the tibial resection guide and the talus resection guide relative to a mechanical axis of the tibia.

13. The system of claim 8, wherein:
the tibial resection guide comprises a tibial bone engagement feature comprising a bone engagement surface configured to register to a surface of the tibia; and
the talus resection guide comprises a talus bone engagement feature comprising a bone engagement surface configured to register to a surface of the talus.

14. The system of claim 13, wherein at least one of the tibial bone engagement feature and the talus bone engagement feature comprise a body section that is coupled to and supports the bone engagement surface and wherein the body section is configured based at least partially on user directions.

15. The system of claim 8, further comprising a positioning guide configured to cooperate with one of the tibial bone attachment feature and the talus bone attachment feature to abut the first osteotomy against the second osteotomy in a stable relationship to close the resection interface.

16. The system of claim 8, further comprising a set of stops configured to prevent the cutting tool from cutting tissue beyond a boundary defined at least partially using the bone model.

17. The system of claim 16, wherein the system includes the set of stops based on user directions.

18. The system of claim 8, wherein one of the tibial resection guide and the talus resection guide comprise a fastener guide configured to guide a fixation system that fixes the tibia to the talus.

19. A method for remediating a bone condition present in a patient's ankle, the method comprising:
positioning a tibial resection guide onto an anterior surface of a distal end of a tibia, the tibial resection guide comprising:
a body having an anterior side, a posterior side, a medial side, a lateral side, a superior side, and an inferior side;
a tibia resection feature configured to guide a cutting tool to prepare the tibia for fusion to a talus;
a tibial bone attachment feature configured to secure the tibial resection guide to the tibia;
a bone engagement feature comprising a bone engagement surface configured to at least partially match a contour of a portion of the anterior surface of the distal end of the tibia when the tibial resection guide is positioned for use;
wherein the tibial resection guide is defined based at least partially on user directions and at least partially on a bone model of at least a portion of the tibia, the bone model based on medical imaging of a patient's foot;
deploying a set of fasteners as part of the tibial bone attachment feature to secure the tibial resection guide to the tibia;
deploying an alignment guide that includes a shaft directed towards a proximal end of the tibia;
inserting the cutting tool into the tibia resection feature and cutting the tibia to form a first osteotomy;
positioning a talus resection guide onto an anterior surface of the proximal end of the talus, the talus resection guide comprising:
a body having an anterior side, a posterior side, a medial side, a lateral side, a superior side, and an inferior side;
a talus resection feature configured to guide the cutting tool to prepare the talus for fusion to the tibia;
a talus bone attachment feature configured to secure the talus resection guide to the talus;
a bone engagement feature comprising a bone engagement surface configured to at least partially match a contour of a portion of the anterior surface of the proximal end of the talus when the talus resection guide is positioned for use;
wherein the talus resection guide is defined based at least partially on user directions and at least partially on a bone model of at least a portion of the talus, the bone model based on medical imaging of the patient's foot;
deploying a set of fasteners as part of the talus bone attachment feature to secure the talus resection guide to the talus;
inserting the cutting tool into the talus resection feature and cutting the talus to form a second osteotomy; and
deploying fixation across the first osteotomy and the second osteotomy to enable fusion of the tibia and the talus.

20. The method of claim 19, further comprising:
accessing an anterior surface of a distal end of a tibia and an anterior surface of a proximal end of a talus of a patient's foot;
deploying a set of stops within the tibia resection feature to manage the cutting tool;
verifying a position of the tibial resection guide by comparing the shaft to a mechanical axis of the tibia and a set of fasteners deployed using the tibial resection guide; and
reducing the first osteotomy and the second osteotomy by abutting a resected distal end of the tibia and a resected proximal end of the talus.

21. A system for an ankle fusion procedure to remediate a bone condition present in a patient's foot, comprising:
a first resection guide comprising:
a body having an anterior side, a posterior side, a medial side, a lateral side, a superior side, and an inferior side;
a first resection feature configured to guide a cutting tool to form a curved osteotomy in a sagittal plane of an ankle of a patient, the curved osteotomy at least partially determined based on a bone model of at least a portion of a patient's ankle, the bone model based on medical imaging of the patient's ankle;
a first bone attachment feature configured to secure the first resection guide to at least one bone of the patient; and
a second resection guide comprising:
a body having an anterior side, a posterior side, a medial side, a lateral side, a superior side, and an inferior side;
a second resection feature configured to guide a cutting tool to form an angled curved osteotomy, the angled curved osteotomy having a curve in a sagittal plane of an ankle of a patient that extends at a first angle in a frontal plane of the ankle that is not perpendicular to a mechanical axis of a tibia of the patient, the angled curved osteotomy at least partially determined based on a bone model of at least a portion of the patient's ankle, the bone model based on medical imaging of the patient's ankle; and
a second bone attachment feature configured to secure the second resection guide to at least one bone of the patient.

22. The system of claim 21, wherein the curved osteotomy extends in the frontal plane at a second angle that is perpendicular to the mechanical axis of the tibia of the patient and wherein the first angle and the second angle are determined at least partially based on the bone model of the portion of the patient's ankle.

23. The system of claim 22, wherein the first angle and the second angle are confirmed by a surgeon.

24. The system of claim 22, wherein the first angle and the second angle are configured such that reduction of the curved osteotomy and the angled curved osteotomy remediates a deformity of the patient.

25. The system of claim 22, wherein the first resection feature extends perpendicularly from the medial side of the first resection guide to the lateral side of the first resection guide at the second angle and the second resection feature extends from the medial side of the second resection guide to the lateral side of the second resection guide at the first angle.

26. The system of claim 21, wherein at least one of the first resection feature and the second resection feature comprises one of a curved slot and a plurality of openings arranged in a curved pattern.

27. The system of claim 21, wherein at least one of the first resection guide and the second resection guide comprises a bone engagement feature comprising:
an insert configured to extend into the curved osteotomy; and
a bone engagement surface configured to engage with bone on one or both sides of the curved osteotomy.

28. The system of claim 21, wherein at least one of the first resection guide and the second resection guide comprises a first bone engagement surface on one side and a second bone engagement surface on another side.
